(19) United States
(12) Patent Application Publication
(10) Pub. No.: US 2024/0247062 A1
(43) Pub. Date: Jul. 25, 2024

(54) HETERODIMERIC ANTIBODIES THAT BIND CLAUDIN18.2 AND CD3

(71) Applicant: Xencor, Inc., Pasadena, CA (US)

(72) Inventors: Matthew J. Bernett, Monrovia, CA (US); Gregory Moore, Azusa, CA (US); Alex Nisthal, Monrovia, CA (US)

(21) Appl. No.: 18/564,994

(22) PCT Filed: Jun. 15, 2022
(86) PCT No.: PCT/US22/33625

§ 371 Date: Nov. 28, 2023

Related U.S. Application Data
(60) Provisional application No. 63/210,787, filed on Jun. 15, 2021.

(51) Int. Cl. C07K 16/28

(52) U.S. Cl. CPC C07K 16/2809; C07K 16/28; C07K 2317/24; C07K 2317/31; C07K 2317/622

(57) ABSTRACT

The present invention is directed to antibodies, including novel antigen binding domains and heterodimeric antibodies, that bind Claudin18.2 (CLDN18.2).

### PRIORITY

This application claims priority to U.S. Provisional Patent Application No. 63/210,787, filed Jun. 15, 2021, which is incorporated herein by reference in its entirety for all purposes.

### SEQUENCE LISTING

The instant application contains a sequence listing which has been submitted electronically in ASCII format and is hereby incorporated by reference in its entirety. Said ASCII copy, created on Jun. 13, 2022, is named 067461-5232-WO_SL.txt and is 1,371,138 bytes in size.

### BACKGROUND OF THE INVENTION

Antibody-based therapeutics are used successfully to treat a variety of diseases, including cancer. An increasingly prevalent avenue being explored is the engineering of single immunoglobulin molecules that co-engage two different antigens. Such alternate antibody formats that engage two different antigens are often referred to as bispecific antibodies. Because the considerable diversity of the antibody variable region (Fv) makes it possible to produce an Fv that recognizes virtually any molecule, the typical approach to bispecific antibody generation is the introduction of new variable regions into the antibody.

A particularly useful approach for bispecific antibodies is to engineer a first binding domain which engages CD3 and a second binding domain which engages an antigen associated with or upregulated on cancer cells so that the bispecific antibody redirects CD3+ T cells to destroy the cancer cells.

Claudins are a family of integral tight junction membrane proteins. Claudin 18 (or CLDN18) is one such protein in the claudin family. Claudin 18 may be expressed as two different splice variants. The claudin 18 isoform A2 splice variant (or CLDN18.2) is expressed on gastric cells, and in particular the cells of stomach epithelium. Notably, it has also been previously reported that CLDN18.2 is highly expressed in several cancers including gastric, esophageal, and pancreatic cancers. In view of this, anti-CLDN18.2 antibodies are useful, for example, for localizing anti-tumor therapeutics (e.g., chemotherapeutic agents and T cells) to such CLDN18.2 expressing tumors.

The present invention provides novel bispecific antibodies to CD3 and CLDN18.2 that are capable of localizing CD3+ effector T cells to CLDN18.2 expressing tumors.

### BRIEF SUMMARY OF THE INVENTION

Accordingly, provided herein are Claudin18.2 (CLDN18.2) antigen binding domains and anti-CLDN18.2 antibodies (e.g., bispecific antibodies).

In one aspect, provided herein is a heterodimeric antibody that includes a first monomer, a second monomer and a common light chain. The first monomer comprises, from N- to C-terminal, VH-CH1-domain linker-scFv-domain linker-CH2-CH3, wherein said CH2-CH3 is a first variant Fc domain. The second monomer comprises, from N- to C-terminal, VH-CH1-hinge-CH2-CH3, wherein said CH2-CH3 is a second variant Fc domain. The common light chain comprises VL-CL. In such antibodies, one of said variant Fc domains comprises amino acid substitutions N208D/Q295E/N384D/Q418E/N421D, one of first and second variant Fc domains each comprise amino acid substitutions E233P/L234V/L235A/G236del/S267K, and one of said variant Fc domains comprises amino acid substitutions S364K/E357Q and the other variant Fc domain comprises amino acid substitutions L368D/K370S, wherein numbering is according to the EU index as in Kabat. The VH domain comprises SEQ ID NO:81 or SEQ ID NO:82 and said VL domain comprises SEQ ID NO:84. In some embodiments, the scFv has a sequence selected from the group consisting of SEQ ID NO:87, SEQ ID NO:88, SEQ ID NO:98, SEQ ID NO:99, SEQ ID NO:109, SEQ ID NO:110, SEQ ID NO:120, SEQ ID NO:121, SEQ ID NO:131, SEQ ID NO:132, SEQ ID NO:142, SEQ ID NO:143.

In some embodiments, the antibody is selected from the group consisting of XENP24647, XENP31729, XENP24649, XENP31723, XENP31725, XENP31727, XENP29476, XENP29478, XENP31724, XENP31726, XENP31728, XENP29477, XENP29479, XENC10101, XENC10102, XENC10103, XENC10104, XENC10105, XENC10106, XENC10107, XENC10108, XENC10109, XENC10110, XENC10111, XENC10112, XENC10113, XENC10114, XENC10115, XENC10116, XENC10117, XENC10118, XENC10119, XENC10120, XENC10121, XENC10122, XENC10123, XENC10124, XENC10125, XENC10126, XENC10127, XENC10128, XENC10129, XENC10130, XENC10131, XENC10132, XENC10133, XENC10134, XENC10135, XENC10136, XENC10137, XENC10138, XENC10139, XENC10140, XENC10141, XENC10142, XENC10143, XENC10144, XENC10145, XENC10146, XENC10147, XENC10148, XENC10149, XENC10150, XENC10151, XENC10152, XENC10153, XENC10154, XENC10155 and XENC10156.

In another aspect, provide herein is a composition comprising an anti-CLDN18.2 antigen binding domain (ABD), wherein said ABD comprises: a) a variable heavy domain comprising SEQ ID NO:81; and b) a variable light domain comprising SEQ ID NO:84.

In another aspect, provide herein is a composition comprising an anti-CLDN18.2 antigen binding domain (ABD), wherein said ABD comprises: a) a variable heavy domain comprising SEQ ID NO:82; and b) a variable light domain comprising SEQ ID NO:84.

In one aspect, provided herein is a heterodimeric antibody that includes a first monomer, a second monomer, and a third monomer. The first monomer includes, from N- to C-terminal, scFv-domain linker-CH2-CH3, wherein said CH2-CH3 is a first variant Fc domain. The second monomer includes, from N- to C-terminal, VH-CH1-hinge-CH2-CH3, wherein said CH2-CH3 is a second variant Fc domain. The third monomer includes VL-CL. In such antibodies, one of said variant Fc domains comprises amino acid substitutions N208D/Q295E/N384D/Q418E/N421D, one of said first and second variant Fc domains each comprise amino acid substitutions E233P/L234V/L235A/G236del/S267K, and one of said variant Fc domains comprises amino acid substitutions S364K/E357Q and the other variant Fc domain comprises amino acid substitutions L368D/K370S, wherein numbering is according to the EU index as in Kabat. In some embodiments, the scFv has a sequence selected from the group consisting of SEQ ID NO:87, SEQ ID NO:88, SEQ ID NO:98, SEQ ID NO:99, SEQ ID NO:109, SEQ ID NO:110, SEQ ID NO:120, SEQ ID NO:121, SEQ ID NO:131, SEQ ID NO:132, SEQ ID NO:142, SEQ ID NO:143. In some embodiments, said variable heavy (VH) domain comprises SEQ ID NO:81 or SEQ ID NO:82 and said variable light (VI) domain comprises SEQ ID NO:84.

In some embodiments, the CH1-hinge-CH2-CH3 component of the second heavy chain comprises SEQ ID NO:32, said first variant Fc domain comprises SEQ ID NO:33 and said constant light domain comprises SEQ ID NO:74. In some embodiments, the antibody is selected from the group consisting of XENP29472, XENP29473, XENP29474 and XENP29475.

In another aspect, provided herein is a heterodimeric antibody comprising a first monomer, a second monomer and a common light chain. The first monomer comprises, from N- to C-terminal, VH1-CH1-hinge-CH2-CH3-domain linker-VH2, wherein said CH2-CH3 is a first variant Fc domain. The second monomer comprises, from N- to C-terminal, VH1-CH1-hinge-CH2-CH3-domain linker-VL2, wherein said CH2-CH3 is a second variant Fc domain. The common light chain comprises a VL1-CL. In such antibodies, one of said variant Fc domains comprises amino acid substitutions N208D/Q295E/N384D/Q418E/N421D, said first and second variant Fc domains each comprise amino acid substitutions E233P/L234V/L235A/G236del/S267K; and one of said variant Fc domains comprises amino acid substitutions S364K/E357Q and the other Fc domain comprises amino acid substitutions L368D/K370S, wherein numbering is according to the EU index as in Kabat. VH2 and VL2 are selected from the pairs consisting of SEQ ID NO:89 and SEQ ID NO:93, SEQ ID NO:100 and SEQ ID NO:104, SEQ ID NO:111 and SEQ ID NO:115, SEQ ID NO:122 and SEQ ID NO:126, SEQ ID NO: 133 and SEQ ID NO:137, SEQ ID NO:144 and SEQ ID NO:148. Further, the VH1 domain comprises SEQ ID NO:81 or SEQ ID NO:82 and said VL1 domain comprises SEQ ID NO:84.

In another aspect, provided herein is a heterodimeric antibody comprising a first monomer, a second monomer and a common light chain. The first monomer comprises, from N- to C-terminal, VH1-CH1-hinge-CH2-CH3-domain linker-scFv, wherein said CH2-CH3 is a first variant Fc domain. The second monomer comprises, from N- to C-terminal, VH1-CH1-hinge-CH2-CH3, wherein said CH2-CH3 is a second variant Fc domain. The common light chain comprises a VL1-CL. In such antibodies, one of said variant Fc domains comprises amino acid substitutions N208D/Q295E/N384D/Q418E/N421D, said first and second variant Fc domains each comprise amino acid substitutions E233P/L234V/L235A/G236del/S267K; and one of said variant Fc domains comprises amino acid substitutions S364K/E357Q and the other Fc domain comprises amino acid substitutions L368D/K370S, wherein numbering is according to the EU index as in Kabat. The scFv has a sequence selected from the group consisting of SEQ ID NO:87, SEQ ID NO:88, SEQ ID NO:98, SEQ ID NO:99, SEQ ID NO:109, SEQ ID NO:110, SEQ ID NO:120, SEQ ID NO:121, SEQ ID NO:131, SEQ ID NO:132, SEQ ID NO:142, SEQ ID NO:143. Further, said VH1 domain comprises SEQ ID NO:81 or SEQ ID NO:82 and said VL1 domain comprises SEQ ID NO:84.

In one aspect, provided herein is a heterodimeric antibody comprising a first monomer, a second monomer and a common light chain. The first monomer comprises, from N- to C-terminal, VH1-CH1-domain linker-VH2-domain linker-CH2-CH3, wherein said CH2-CH3 is a first variant Fc domain. The second monomer comprising, from N- to C-terminal, VH1-CH1-domain linker-VL2-domain linker-CH2-CH3, wherein said CH2-CH3 is a second variant Fc domain. The common light chain comprising a VL1-CL. In such antibodies, one of said variant Fc domains comprises amino acid substitutions N208D/Q295E/N384D/Q418E/N421D, said first and second variant Fc domains each comprise amino acid substitutions E233P/L234V/L235A/G236del/S267K; and one of said variant Fc domains comprises amino acid substitutions S364K/E357Q and the other Fc domain comprises amino acid substitutions L368D/K370S, wherein numbering is according to the EU index as in Kabat. The VH2 and VL2 are selected from the pairs consisting of SEQ ID NO:89 and SEQ ID NO:93, SEQ ID NO:100 and SEQ ID NO:104, SEQ ID NO:111 and SEQ ID NO:115, SEQ ID NO:122 and SEQ ID NO:126, SEQ ID NO: 133 and SEQ ID NO:137, SEQ ID NO:144 and SEQ ID NO:148. Further, said VH1 domain comprises SEQ ID NO:81 or SEQ ID NO:82 and said VL1 domain comprises SEQ ID NO:84

In another aspect, provided herein is a heterodimeric antibody comprising a first monomer, a second monomer and a light chain. The first monomer comprises, from N- to C-terminal, VH-CH1-domain linker-scFv-domain linker-CH2-CH3, wherein said CH2-CH3 is a first variant Fc domain. The second monomer comprises a second variant Fc domain comprising CH2-CH3. The light chain comprises VL-CL. In such antibodies, one of said variant Fc domains comprises amino acid substitutions N208D/Q295E/N384D/Q418E/N421D, said first and second variant Fc domains each comprise amino acid substitutions E233P/L234V/L235A/G236del/S267K, and one of said variant Fc domains comprises amino acid substitutions S364K/E357Q and the other variant Fc domain comprises amino acid substitutions L368D/K370S, wherein numbering is according to the EU index as in Kabat. The scFv has a sequence selected from the group consisting of SEQ ID NO:87, SEQ ID NO:88, SEQ ID NO:98, SEQ ID NO:99, SEQ ID NO:109, SEQ ID NO:110, SEQ ID NO:120, SEQ ID NO:121, SEQ ID NO:131, SEQ ID NO:132, SEQ ID NO:142, SEQ ID NO:143. Further, said VH domain comprises SEQ ID NO:81 or SEQ ID NO:82 and said VL domain comprises SEQ ID NO:84

In one aspect, provided herein is a heterodimeric antibody comprising a first monomer, a second monomer and a light chain. The first monomer comprises, from N- to C-terminal, scFv-domain linker-VH-CH1-hinge-CH2-CH3, wherein said CH2-CH3 is a first variant Fc domain. The second monomer comprises a second variant Fc domain comprising CH2-CH3. The light chain comprises VL-CL. In such antibodies, one of said variant Fc domains comprises amino acid substitutions N208D/Q295E/N384D/Q418E/N421D, said first and second variant Fc domains each comprise amino acid substitutions E233P/L234V/L235A/G236del/S267K, and one of said variant Fc domains comprises amino acid substitutions S364K/E357Q and the other variant Fc domain comprises amino acid substitutions L368D/K370S, wherein numbering is according to the EU index as in Kabat. The scFv has a sequence selected from the group consisting of SEQ ID NO:87, SEQ ID NO:88, SEQ ID NO:98, SEQ ID NO:99, SEQ ID NO:109, SEQ ID NO:110, SEQ ID NO:120, SEQ ID NO:121, SEQ ID NO:131, SEQ ID NO:132, SEQ ID NO:142, SEQ ID NO:143. Further, said VH domain comprises SEQ ID NO:81 or SEQ ID NO:82 and said VL domain comprises SEQ ID NO:84

In another aspect, provided herein is a heterodimeric antibody comprising a first monomer, a second monomer and a common light chain. The first monomer comprises, from N- to C-terminal, scFv-domain linker-VH-CH1-hinge-CH2-CH3, wherein said CH2-CH3 is a first variant Fc domain. The second monomer comprises, from N- to C-terminal, VH-CH1-hinge-CH2-CH3, wherein said CH2-CH3 is a second variant Fc domain. The common light chain comprises VL-CL. In such antibodies, one of said variant Fc domains comprises amino acid substitutions N208D/Q295E/N384D/Q418E/N421D, said first and second variant Fc domains each comprise amino acid substitutions E233P/L234V/L235A/G236del/S267K, and one of said variant Fc domains comprises amino acid substitutions S364K/E357Q and the other variant Fc domain comprises amino acid substitutions L368D/K370S, wherein numbering is according to the EU index as in Kabat. The scFv has a sequence selected from the group consisting of SEQ ID NO:87, SEQ ID NO:88, SEQ ID NO:98, SEQ ID NO:99, SEQ ID NO:109, SEQ ID NO:110, SEQ ID NO:120, SEQ ID NO:121, SEQ ID NO:131, SEQ ID NO:132, SEQ ID NO:142, SEQ ID NO:143. Further, said VH domain comprises SEQ ID NO:81 or SEQ ID NO:82 and said VL domain comprises SEQ ID NO:84

In another aspect, provided herein is a nucleic acid composition that includes nucleic acids encoding any of the heterodimeric antibodies or antigen binding domains described herein.

In yet another aspect, provided herein is an expression vector that includes any of the nucleic acids described herein.

In one aspect, provided herein is a host cell transformed with any of the expression vectors or nucleic acids described herein.

In another aspect, provided herein is a method of making a subject heterodimeric antibody or antigen binding domain described herein. The method includes a step of culturing a host cell transformed with any of the expression vectors or nucleic acids described herein under conditions wherein the antibody or antigen binding domain is expressed, and recovering the antibody or antigen binding domain.

In one aspect, provided herein is a method of treating cancer that includes administering to a patient in need thereof any one of the subject antibodies described herein. In some embodiments, the cancer is a gastric cancer.

### BRIEF DESCRIPTION OF THE DRAWINGS

FIG. 1A-1E depict useful pairs of Fc heterodimerization variant sets (including skew and pI variants). There are variants for which there are no corresponding “monomer 2” variants; these are pI variants which can be used alone on either monomer.

FIG. 2 depicts a list of isosteric variant antibody constant regions and their respective substitutions. pI_(−) indicates lower pI variants, while pI_(+) indicates higher pI variants. These can be optionally and independently combined with other heterodimerization variants of the inventions (and other variant types as well, as outlined herein.)

FIG. 3 depicts useful ablation variants that ablate FcγR binding (sometimes referred to as “knock outs” or “KO” variants). Generally, ablation variants are found on both monomers, although in some cases they may be on only one monomer.

FIG. 4 depicts particularly useful embodiments of “non-Fv” components of the invention.

FIG. 5 depict a number of charged scFv linkers that find use in increasing or decreasing the pI of heterodimeric antibodies that utilize one or more scFv as a component. The (+H) positive linker finds particular use herein. A single prior art scFv linker with single charge is referenced as “Whitlow”, from Whitlow et al., Protein Engineering 6(8):989-995 (1993). It should be noted that this linker was used for reducing aggregation and enhancing proteolytic stability in scFvs. It should also be noted that any or all of these linkers can be used as optional domain linkers as discussed herein, and in particular, those listed as “Additional scFv linkers” that are uncharged may find particular use.

FIG. 6A-6D depicts the sequences of several useful 1+1 Fab-scFv-Fc bispecific antibody format heavy chain backbones based on human IgG1, without the Fv sequences (e.g. the scFv and the VH for the Fab side). That is, the “/” slash at the beginning of the “Fab-Fc side” indicates that the C-terminus of a VH as outlined herein is attached at that point. Similarly, “/” slash at the beginning of the “scFv-Fc side” indicates that the C-terminus of a scFv (e.g. VH-scFv linker-VL or VL-scFv linker-VH) as outlined herein is attached at that point. Backbone 1 is based on human IgG1 (356E/358M allotype), and includes the S364K/E357Q:L368D/K370S skew variants, C220S on the chain with the S364K/E357Q skew variants, the N208D/Q295E/N384D/Q418E/N421D pI variants on the chain with L368D/K370S skew variants and the E233P/L234V/L235A/G236del/S267K ablation variants on both chains. Backbone 2 is based on human IgG1 (356E/358M allotype), and includes S364K:L368D/K370S skew variants, C220S on the chain with the S364K skew variant, the N208D/Q295E/N384D/Q418E/N421D pI variants on the chain with L368D/K370S skew variants, and the E233P/L234V/L235A/G236del/S267K ablation variants on both chains. Backbone 3 is based on human IgG1 (356E/358M allotype), and includes S364K:L368E/K370S skew variants, C220S on the chain with the S364K skew variant, the N208D/Q295E/N384D/Q418E/N421D pI variants on the chain with L368E/K370S skew variants and the E233P/L234V/L235A/G236del/S267K ablation variants on both chains. Backbone 4 is based on human IgG1 (356E/358M allotype), and includes D401K:K360E/Q362E/T411E skew variants, C220S on the chain with the D401K skew variant, the N208D/Q295E/N384D/Q418E/N421D pI variants on the chain with K360E/Q362E/T411E skew variants and the E233P/L234V/L235A/G236del/S267K ablation variants on both chains. Backbone 5 is based on human IgG1 (356D/358L allotype), and includes S364K/E357Q:L368D/K370S skew variants, C220S on the chain with the S364K/E357Q skew variants, the N208D/Q295E/N384D/Q418E/N421D pI variants on the chain with L368D/K370S skew variants and the E233P/L234V/L235A/G236del/S267K ablation variants on both chains. Backbone 6 is based on human IgG1 (356E/358M allotype), and includes S364K/E357Q:L368D/K370S skew variants, C220S on the chain with the S364K/E357Q skew variants, N208D/Q295E/N384D/Q418E/N421D pI variants on the chain with L368D/K370S skew variants and the E233P/L234V/L235A/G236del/S267K ablation variants on both chains, as well as an N297A variant on both chains. Backbone 7 is identical to 6 except the mutation is N297S. Backbone 8 is based on human IgG4, and includes the S364K/E357Q:L368D/K370S skew variants, the N208D/Q295E/N384D/Q418E/N421D pI variants on the chain with L368D/K370S skew variants, as well as a S228P (EU numbering, this is S241P in Kabat) variant on both chains that ablates Fab arm exchange as is known in the art. Backbone 9 is based on human IgG2, and includes the S364K/E357Q:L368D/K370S skew variants, the N208D/Q295E/N384D/Q418E/N421D pI variants on the chain with L368D/K370S skew variants. Backbone 10 is based on human IgG2, and includes the S364K/E357Q:L368D/K370S skew variants, the N208D/Q295E/N384D/Q418E/N421D pI variants on the chain with L368D/K370S skew variants as well as a S267K variant on both chains. Backbone 11 is identical to backbone 1, except it includes M428L/N434S Xtend mutations. Backbone 12 is based on human IgG1 (356E/358M allotype), and includes S364K/E357Q:L368D/K370S skew variants, C220S and the P217R/P229R/N276K pI variants on the chain with S364K/E357Q skew variants and the E233P/L234V/L235A/G236del/S267K ablation variants on both chains. Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1 (or IgG2 or IgG4, depending on the backbone). That is, the recited backbones may contain additional amino acid modifications (generally amino acid substitutions) in addition to the skew, pI and ablation variants contained within the backbones of this figure.

FIG. 7A-7C depicts the sequences of several useful 2+1 Fab₂-scFv-Fc bispecific antibody format heavy chain backbones based on human IgG1, without the sequences of the Fvs and linkers. Thus, for example, on the “Fab-Fc side”, the (VH domain/” indicates that the C-terminus of a VH domain is attached at that location, which is the beginning of the CH1 domain of a heavy chain. Similarly, the “VH-CH1-domain linker1-scFv-domain linker2/” at the beginning of the “Fab-scFv-Fc side” indicates that these sequences are attached to the recited sequence, which is the variant CH2-CH3 Fc domain. As discussed herein, the scFv domain can be in either orientation, -VH-scFv linker-VL- or -VL-scFv linker-VH- as discussed herein. Additionally, preferred domain linkers for “domain linker2” are those recited in FIG. 5 as “useful domain linkers”. Note that the sequence identifiers are only to the recited sequences. Backbone 1 is based on human IgG1 (356E/358M allotype), and includes the S364K/E357Q:L368D/K370S skew variants, the N208D/Q295E/N384D/Q418E/N421D pI variants on the chain with L368D/K370S skew variants and the E233P/L234V/L235A/G236del/S267K ablation variants on both chains. Backbone 2 is based on human IgG1 (356E/358M allotype), and includes S364K:L368D/K370S skew variants, the N208D/Q295E/N384D/Q418E/N421D pI variants on the chain with L368D/K370S skew variants, and the E233P/L234V/L235A/G236del/S267K ablation variants on both chains. Backbone 3 is based on human IgG1 (356E/358M allotype), and includes S364K:L368E/K370S skew variants, the N208D/Q295E/N384D/Q418E/N421D pI variants on the chain with L368E/K370S skew variants and the E233P/L234V/L235A/G236del/S267K ablation variants on both chains. Backbone 4 is based on human IgG1 (356E/358M allotype), and includes D401K:K360E/Q362E/T411E skew variants, the N208D/Q295E/N384D/Q418E/N421D pI variants on the chain with K360E/Q362E/T411E skew variants and the E233P/L234V/L235A/G236del/S267K ablation variants on both chains. Backbone 5 is based on human IgG1 (356D/358L allotype), and includes S364K/E357Q:L368D/K370S skew variants, the N208D/Q295E/N384D/Q418E/N421D pI variants on the chain with L368D/K370S skew variants and the E233P/L234V/L235A/G236del/S267K ablation variants on both chains. Backbone 6 is based on human IgG1 (356E/358M allotype), and includes S364K/E357Q:L368D/K370S skew variants, N208D/Q295E/N384D/Q418E/N421D pI variants on the chain with L368D/K370S skew variants and the E233P/L234V/L235A/G236del/S267K ablation variants on both chains, as well as an N297A variant on both chains. Backbone 7 is identical to 6 except the mutation is N297S. Backbone 8 is identical to backbone 1, except it includes M428L/N434S Xtend mutations. Backbone 9 is based on human IgG1 (356E/358M allotype), and includes S364K/E357Q:L368D/K370S skew variants, the P217R/P229R/N276K pI variants on the chain with S364K/E357Q skew variants and the E233P/L234V/L235A/G236del/S267K ablation variants on both chains. Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1 (or IgG2 or IgG4, depending on the backbone). That is, the recited backbones may contain additional amino acid modifications (generally amino acid substitutions) in addition to the skew, pI and ablation variants contained within the backbones of this figure.

FIG. 8 depicts the “non-Fv” backbone of cognate light chains (i.e. constant light chain) which find use in the 1+1 Fab-scFv-Fc and 2+1 Fab₂-scFv-Fc bispecific antibodies of the invention.

FIG. 9 depicts the sequences for A) human claudin 18 isoform A2 (CLDN18.2), B) mouse claudin 18 isoform A2.1, C) mouse claudin 18 isoform A2.2, and D) cynomolgus claudin 18, to facilitate the development of antigen binding domains that are cross-reactive for ease of clinical development.

FIG. 10 depicts the variable heavy and variable light chains for illustrative anti-CLDN18.2 ABDs which find use in the anti-CLDN18.2x anti-CD3 bispecific antibodies of the invention. The CDRs are underlined. As noted herein and is true for every sequence herein containing CDRs, the exact identification of the CDR locations may be slightly different depending on the numbering used as is shown in Table 1, and thus included herein are not only the CDRs that are underlined but also CDRs included within the V_{H} and V_{L} domains using other numbering systems. H0 Variable Heavy and L0 Variable Light are murine; and H1 Variable Heavy, H2 Variable Heavy, and L1 Variable Light are humanized.

FIG. 11 depicts sequences for a murine anti-CLDN18.2 antibody with an ablation variant (E233P/L234V/L235A/G236del/S267K, “IgG1_PVA_/S267K”). The CDRs are underlined. As noted herein and is true for every sequence herein containing CDRs, the exact identification of the CDR locations may be slightly different depending on the numbering used as is shown in Table 1, and thus included herein are not only the CDRs that are underlined but also CDRs included within the V_{H} and V_{L} domains using other numbering systems.

FIG. 12A-12F depicts sequences for exemplary anti-CD3 scFvs suitable for use in the bispecific antibodies of the invention. The CDRs are underlined, the scFv linker is double underlined (in the sequences, the scFv linker is a positively charged scFv (GKPGS)₄ linker (SEQ ID NO: 10), although as will be appreciated by those in the art, this linker can be replaced by other linkers, including uncharged or negatively charged linkers, some of which are depicted in FIG. 5), and the slashes indicate the border(s) of the variable domains. In addition, the naming convention illustrates the orientation of the scFv from N- to C-terminus. The scFv sequences are shown in both orientations as indicated. As noted herein and is true for every sequence herein containing CDRs, the exact identification of the CDR locations may be slightly different depending on the numbering used as is shown in Table 1, and thus included herein are not only the CDRs that are underlined but also CDRs included within the V_{H} and V_{L} domains using other numbering systems. Furthermore, as for all the sequences in the Figures, these V_{H} and V_{L} sequences can be used either in a scFv format or in a Fab format.

FIG. 13A-13B depicts a couple of useful formats of the present invention. FIG. 13A depicts the “1+1 Fab-scFv-Fc” format, with a first Fab arm binding CLDN18.2 and a second scFv arm binding CD3. FIG. 13B depicts the “2+1 Fab₂-scFv-Fc” format, with a first Fab arm binding CLDN18.2 and a second Fab-scFv arm, wherein the Fab binds CLDN18.2 and the scFv binds CD3. It should be noted that additional formats can be used as well, as generally depicted in FIG. 42, herein.

FIG. 14 depicts the amino acid sequences of prototype anti-CLDN18.2x anti-CD3 bispecific antibodies (based on murine CLDN18.2 ABD as depicted in FIG. 10) in the 1+1 Fab-scFv-Fc format. The antibodies are named using the Fab variable region first and the scFv variable region second, separated by a dash. CDRs are underlined and slashes indicate the border(s) of the variable regions. The scFv domain has orientation (N- to C-terminus) of V_{H}-ScFv linker-V_{L}, although this can be reversed. In addition, each sequence outlined herein can include or exclude the M428L/N434S variants in one or preferably both Fc domains, which results in longer half-life in serum.

FIG. 15A-15AQ depicts the amino acid sequences of prototype anti-CLDN18.2 x anti-CD3 bispecific antibodies (based on murine CLDN18.2 ABD as depicted in FIG. 10) in the 2+1 Fab₂-scFv-Fc format. The antibodies are named using the Fab variable region first and the Fab-scFv variable regions second, separated by a dash. CDRs are underlined and slashes indicate the border(s) of the variable regions. The scFv domain has orientation (N- to C-terminus) of V_{H}-ScFv linker-V_{L}, although this can be reversed. In addition, each sequence outlined herein can include or exclude the M428L/N434S variants in one or preferably both Fc domains, which results in longer half-life in serum.

FIG. 16 depicts the amino acid sequences of a control anti-RSV x anti-CD3 bispecific antibodies in the 1+1 Fab-scFv-Fc format. The antibody is named using the Fab variable region first and the scFv variable region second, separated by a dash. CDRs are underlined and slashes indicate the border(s) of the variable regions. The scFv domain has orientation (N- to C-terminus) of V_{H}-ScFv linker-V_{L}, although this can be reversed. In addition, each sequence outlined herein can include or exclude the M428L/N434S variants in one or preferably both Fc domains, which results in longer half-life in serum.

FIG. 17A-17B depicts binding of prototype anti-CLDN18.2 x anti-CD3 bispecific antibodies having murine CLDN18.2 ABD XENP24645 (1+1 Fab-scFv-Fc with CD3-High), XENP24646 (1+1 Fab-scFv-Fc with CD3-High-Int #1), XENP24647 (2+1 Fab₂-scFv-Fc with CD3-High), XENP24648 (2+1 Fab₂-scFv-Fc with CD3-High Int #1), and XENP24649 (2+1 Fab₂-scFv-Fc with CD3-Intermediate) to A) KP-4 cells and B) NUGC-4 cells. Controls included an anti-RSV x anti-CD3 bispecific antibody, cells only, and secondary antibody only. The data show that the prototype anti-CLDN18.2 x anti-CD3 bsAbs dose-dependently bound to NUGC-4 cells, with close to baseline binding to KP-4 cells at all concentrations tested. Notably, bsAbs of the “2+1 Fab₂-scFv-Fc” format (i.e. XENP24647, XENP24648, and XENP24949) bound much more potently to NUGC-4 cells than did bsAbs of the “1+1 Fab-scFv-Fc” format (i.e. XENP24645 and XENP24646) due to the extra avidity conveyed by the “2+1 Fab₂-scFv-Fc” format.

FIG. 18A-18B depicts induction of RTCC on A) KP-4 cells and B) NUGC-4 cells by prototype anti-CLDN18.2 x anti-CD3 bispecific antibodies having murine CLDN18.2 ABD XENP24645 (1+1 Fab-scFv-Fc with CD3-High), XENP24646 (1+1 Fab-scFv-Fc with CD3-High-Int #1), XENP24647 (2+1 Fab₂-scFv-Fc with CD3-High), XENP24648 (2+1 Fab₂-scFv-Fc with CD3-High Int #1), and XENP24649 (2+1 Fab₂-scFv-Fc with CD3-Intermediate). The data show that the prototype anti-CLDN18.2 x anti-CD3 bsAbs dose-dependently induced RTCC on NUGC-4, and no RTCC on KP-4 cells.

FIG. 19A-19B depicts the expression levels on A) NUGC-4 cells and B) SNU-601 cells as determined by flow cytometry. The data indicate that SNU-601 expresses more CLDN18.2 than does NUGC-4.

FIG. 20A-20B depicts binding of prototype anti-CLDN18.2 x anti-CD3 bispecific antibodies having murine CLDN18.2 ABD XENP24645 (1+1 Fab-scFv-Fc with CD3-High), XENP24646 (1+1 Fab-scFv-Fc with CD3-High-Int #1), XENP24647 (2+1 Fab₂-scFv-Fc with CD3-High), and XENP24649 (2+1 Fab₂-scFv-Fc with CD3-Intermediate) to A) NUGC-4 cells and B) SNU-601 cells. Controls included an anti-RSV x anti-CD3 bispecific antibody, cells only, and secondary antibody only. The data show that each of the bsAbs dose-dependently bound to both NUGC-4 and SNU-601, with higher maximal binding to SNU-601 cells than to NUGC-4, which is consistent with the respective CLDN18.2 expression levels on each cell line.

FIG. 21A-21B depicts induction of RTCC (as indicated by decrease in live target cells) on A) NUGC-4 and B) SNU-601 cells after incubation for 24 hours with human PBMCs (20:1 effector to target cell ratio) and the following prototype anti-CLDN18.2 x anti-CD3 bispecific antibodies having murine CLDN18.2 ABD: XENP24645 (1+1 Fab-scFv-Fc with CD3-High), XENP24646 (1+1 Fab-scFv-Fc with CD3-High-Int #1), XENP24647 (2+1 Fab₂-scFv-Fc with CD3-High), and XENP24649 (2+1 Fab₂-scFv-Fc with CD3-Intermediate). Controls included an anti-RSV x anti-CD3 bispecific antibody, target cells only, and target cells and effector cells only. The data show that the bsAbs enhance killing of target cells (i.e. NUGC-4 and SNU-601 cells) as indicated by decrease in live cells.

FIG. 22A-22B depicts induction of RTCC (as indicated by increase in dead target cells) on A) NUGC-4 and B) SNU-601 cells after incubation for 24 hours with human PBMCs (20:1 effector to target cell ratio) and the following prototype anti-CLDN18.2 x anti-CD3 bispecific antibodies having murine CLDN18.2 ABD: XENP24645 (1+1 Fab-scFv-Fc with CD3-High), XENP24646 (1+1 Fab-scFv-Fc with CD3-High-Int #1), XENP24647 (2+1 Fab₂-scFv-Fc with CD3-High), XENP24648 (2+1 Fab₂-scFv-Fc with CD3-High Int #1), and XENP24649 (2+1 Fab₂-scFv-Fc with CD3-Intermediate). Controls included an anti-RSV x anti-CD3 bispecific antibody, target cells only, and target cells and effector cells only. The data show that the bsAbs enhance killing of target cells (i.e. NUGC-4 and SNU-601 cells) as indicated by increase in dead/dying cells.

FIG. 23A-23B depicts induction of RTCC (as indicated by decrease in live target cells) on A) NUGC-4 and B) SNU-601 cells after incubation for 48 hours with human PBMCs (20:1 effector to target cell ratio) and the following prototype anti-CLDN18.2 x anti-CD3 bispecific antibodies having murine CLDN18.2 ABD: XENP24645 (1+1 Fab-scFv-Fc with CD3-High), XENP24646 (1+1 Fab-scFv-Fc with CD3-High-Int #1), XENP24647 (2+1 Fab₂-scFv-Fc with CD3-High), and XENP24649 (2+1 Fab₂-scFv-Fc with CD3-Intermediate). Controls included an anti-RSV x anti-CD3 bispecific antibody, target cells only, and target cells and effector cells only. The data show that the bsAbs enhance killing of target cells (i.e. NUGC-4 and SNU-601 cells) as indicated by decrease in live cells.

FIG. 24A-24B depicts induction of RTCC (as indicated by increase in dead target cells) on A) NUGC-4 and B) SNU-601 cells after incubation for 48 hours with human PBMCs (20:1 effector to target cell ratio) and the following prototype anti-CLDN18.2 x anti-CD3 bispecific antibodies having murine CLDN18.2 ABD: XENP24645 (1+1 Fab-scFv-Fc with CD3-High), XENP24646 (1+1 Fab-scFv-Fc with CD3-High-Int #1), XENP24647 (2+1 Fab₂-scFv-Fc with CD3-High), and XENP24649 (2+1 Fab₂-scFv-Fc with CD3-Intermediate). Controls included an anti-RSV x anti-CD3 bispecific antibody, target cells only, and target cells and effector cells only. The data show that the bsAbs enhance killing of target cells (i.e. NUGC-4 and SNU-601 cells) as indicated by increase in dead/dying cells.

FIG. 25A-25C depicts percentage of CD4⁺ T cells expressing A) CD69, B) CD25, and C) CD107a following incubation of NUGC-4 cells for 48 hours with human PBMCs (20:1 effector to target cell ratio) and the following prototype anti-CLDN18.2 x anti-CD3 bispecific antibodies having murine CLDN18.2 ABD: XENP24645 (1+1 Fab-scFv-Fc with CD3-High), XENP24646 (1+1 Fab-scFv-Fc with CD3-High-Int #1), XENP24647 (2+1 Fab₂-scFv-Fc with CD3-High), and XENP24649 (2+1 Fab₂-scFv-Fc with CD3-Intermediate). Controls included an anti-RSV x anti-CD3 bispecific antibody, target cells only, and target cells and effector cells only. The data shows a trend consistent with RTCC, that is for example, higher affinity CD3 binding and/or bivalent CLDN18.2 binding enhance T cell activation and degranulation.

FIG. 26A-26C depicts percentage of CD8⁺ T cells expressing A) CD69, B) CD25, and C) CD107a following incubation of NUGC-4 cells for 48 hours with human PBMCs (20:1 effector to target cell ratio) and the following prototype anti-CLDN18.2 x anti-CD3 bispecific antibodies having murine CLDN18.2 ABD: XENP24645 (1+1 Fab-scFv-Fc with CD3-High), XENP24646 (1+1 Fab-scFv-Fc with CD3-High-Int #1), XENP24647 (2+1 Fab₂-scFv-Fc with CD3-High), and XENP24649 (2+1 Fab₂-scFv-Fc with CD3-Intermediate). Controls included an anti-RSV x anti-CD3 bispecific antibody, target cells only, and target cells and effector cells only. The data shows a trend consistent with RTCC, that is for example, higher affinity CD3 binding and/or bivalent CLDN18.2 binding enhance T cell activation and degranulation.

FIG. 27A-27C depicts percentage of CD4⁺ T cells expressing A) CD69, B) CD25, and C) CD107a following incubation of SNU-601 cells for 48 hours with human PBMCs (20:1 effector to target cell ratio) and the following prototype anti-CLDN18.2 x anti-CD3 bispecific antibodies having murine CLDN18.2 ABD: XENP24645 (1+1 Fab-scFv-Fc with CD3-High), XENP24646 (1+1 Fab-scFv-Fc with CD3-High-Int #1), XENP24647 (2+1 Fab₂-scFv-Fc with CD3-High), and XENP24649 (2+1 Fab₂-scFv-Fc with CD3-Intermediate). Controls included an anti-RSV x anti-CD3 bispecific antibody, target cells only, and target cells and effector cells only. The data shows a trend consistent with RTCC, that is for example, higher affinity CD3 binding and/or bivalent CLDN18.2 binding enhance T cell activation and degranulation.

FIG. 28A-28C depicts percentage of CD8⁺ T cells expressing A) CD69, B) CD25, and C) CD107a following incubation of SNU-601 cells for 48 hours with human PBMCs (20:1 effector to target cell ratio) and the following prototype anti-CLDN18.2 x anti-CD3 bispecific antibodies having murine CLDN18.2 ABD: XENP24645 (1+1 Fab-scFv-Fc with CD3-High), XENP24646 (1+1 Fab-scFv-Fc with CD3-High-Int #1), XENP24647 (2+1 Fab₂-scFv-Fc with CD3-High), and XENP24649 (2+1 Fab₂-scFv-Fc with CD3-Intermediate). Controls included an anti-RSV x anti-CD3 bispecific antibody, target cells only, and target cells and effector cells only. The data shows a trend consistent with RTCC, that is for example, higher affinity CD3 binding and/or bivalent CLDN18.2 binding enhance T cell activation and degranulation.

FIG. 29 depicts sequences for anti-CLDN18.2 antibodies with humanized variable regions with an ablation variant (E233P/L234V/L235A/G236del/S267K, “IgG1_PVA_/S267K”). The CDRs are underlined. As noted herein and is true for every sequence herein containing CDRs, the exact identification of the CDR locations may be slightly different depending on the numbering used as is shown in Table 1, and thus included herein are not only the CDRs that are underlined but also CDRs included within the V_{H} and V_{L} domains using other numbering systems.

FIG. 30 depicts germline identity of humanized CLDN18.2 ABDs in comparison to murine CLDN18.2 ABD.

FIG. 31A-31C depicts the amino acid sequences of anti-CLDN18.2 x anti-CD3 bispecific antibodies with humanized variable regions in the 1+1 Fab-scFv-Fc format. The antibodies are named using the Fab variable region first and the scFv variable region second, separated by a dash. CDRs are underlined and slashes indicate the border(s) of the variable regions. The scFv domain has orientation (N- to C-terminus) of V_{H}-ScFv linker-V_{L}, although this can be reversed. In addition, each sequence outlined herein can include or exclude the M428L/N434S variants in one or preferably both Fc domains, which results in longer half-life in serum.

FIG. 32A-32C depicts the amino acid sequences of anti-CLDN18.2 x anti-CD3 bispecific antibodies with humanized variable regions in the 2+1 Fab₂-scFv-Fc format. The antibodies are named using the Fab variable region first and the Fab-scFv variable regions second, separated by a dash. CDRs are underlined and slashes indicate the border(s) of the variable regions. The scFv domain has orientation (N- to C-terminus) of V_{H}-ScFv linker-V_{L}, although this can be reversed. In addition, each sequence outlined herein can include or exclude the M428L/N434S variants in one or preferably both Fc domains, which results in longer half-life in serum.

FIG. 33A-33B depicts the expression levels on A) SNU-601 cells and B) SNU-601(2E4) cells (enriched for CLDN18.2 expressing population) as determined by flow cytometry. The data show that the SNU-601(2E4) contains substantially higher population of CLDN18.2⁺ cells. Experiments in this section are performed using SNU-601(2E4) cells.

FIG. 34 depicts binding of by the following anti-CLDN18.2 x anti-CD3 bispecific antibodies having humanized CLDN18.2 ABD: XENP29472 (H1L1 CLDN18.2 ABD; 1+1 Fab-scFv-Fc with CD3-High), XENP29473 (H2L1 CLDN18.2 ABD; 1+1 Fab-scFv-Fc with CD3-High), XENP29474 (H1L1 CLDN18.2 ABD; 1+1 Fab-scFv-Fc with CD3-HighInt #1), XENP29475 (H2L1 CLDN18.2 ABD; 1+1 Fab-scFv-Fc with CD3-HighInt #1), XENP29476 (H1L1 CLDN18.2 ABD; 2+1 Fab₂-scFv-Fc with CD3-High), XENP29477 (H2L1 CLDN18.2 ABD; 2+1 Fab₂-scFv-Fc with CD3-High), XENP29478 (H1L1 CLDN18.2 ABD; 2+1 Fab₂-scFv-Fc with CD3-HighInt #1), and XENP29479 (H2L1 CLDN18.2 ABD; 2+1 Fab₂-scFv-Fc with CD3-HighInt #1) to SNU-601(2E4) cells. Controls used were XENP24644 (H0L0 CLDN18.2; bivalent mAb), XENP29470 (H1L1 CLDN18.2; bivalent mAb), XENP29471 (H2L1 CLDN18.2; bivalent mAb), XENP24645 (1+1 Fab-scFv-Fc with CD3-High), XENP24647 (2+1 Fab₂-scFv-Fc with CD3-High), cells only, and secondary antibody only. The data show that the bsAbs having humanized CLDN18.2 ABDs had similar binding to SNU-601(2E4) cells as bsAbs having murine CLDN18.2 ABDs indicating that humanization preserved the binding efficacy of the bsAbs. Notably, bsAbs in the “2+1 Fab2-scFv-Fc” format showed similar binding to bivalent anti-CLDN18.2 mAbs. Additionally, the data show that bsAbs based on the H1L1 humanized variant (e.g. XENP29472, XENP29474, XENP28476, and XENP29478) better preserved binding than did bsAbs based on the H2L1 humanized variant (e.g. XENP29473, XENP29475, XENP29477, and XENP29479).

FIG. 35A-35C depicts induction of RTCC on SNU-601(2E4) cells A) as indicated by decrease in number of CFSE⁺ SNU-601(2E4), B) as indicated by percentage of CFSE⁺ SNU-601(2E4) cells stained with Zombie Aqua, and C) as indicated by Zombie Aqua MFI on CFSE⁺ SNU-601(2E4) cells after incubation of CFSE-labeled SNU-601(2E4) for 24 hours with human PBMCs (10:1 effector to target cell ratio) and the following anti-CLDN18.2 x anti-CD3 bispecific antibodies having humanized CLDN18.2 ABD: XENP29472 (H1L1 CLDN18.2 ABD; 1+1 Fab-scFv-Fc with CD3-High), XENP29473 (H2L1 CLDN18.2 ABD; 1+1 Fab-scFv-Fc with CD3-High), XENP29474 (H1L1 CLDN18.2 ABD; 1+1 Fab-scFv-Fc with CD3-HighInt #1), XENP29475 (H2L1 CLDN18.2 ABD; 1+1 Fab-scFv-Fc with CD3-HighInt #1), XENP29476 (H1L1 CLDN18.2 ABD; 2+1 Fab₂-scFv-Fc with CD3-High), XENP29477 (H2L1 CLDN18.2 ABD; 2+1 Fab₂-scFv-Fc with CD3-High), XENP29478 (H1L1 CLDN18.2 ABD; 2+1 Fab₂-scFv-Fc with CD3-HighInt #1), and XENP29479 (H2L1 CLDN18.2 ABD; 2+1 Fab₂-scFv-Fc with CD3-HighInt #1) to SNU-601(2E4) cells. Controls used were XENP24645 (1+1 Fab-scFv-Fc with CD3-High), XENP24647 (2+1 Fab₂-scFv-Fc with CD3-High), cells only, and secondary antibody only. Consistent with the binding data, humanization preserved the induction of RTCC by the bsAbs having humanized CLDN18.2 ABDs.

FIG. 36A-36B depicts activation CD4⁺ T cells [as indicated by A) CD69 MFI on CD4⁺ T cells, and B) percentage of CD4⁺ T cells expressing CD69] after incubation of SNU-601(2E4) for 24 hours with human PBMCs (10:1 effector to target cell ratio) and the following anti-CLDN18.2 x anti-CD3 bispecific antibodies having humanized CLDN18.2 ABD: XENP29472 (H1L1 CLDN18.2 ABD; 1+1 Fab-scFv-Fc with CD3-High), XENP29473 (H2L1 CLDN18.2 ABD; 1+1 Fab-scFv-Fc with CD3-High), XENP29474 (H1L1 CLDN18.2 ABD; 1+1 Fab-scFv-Fc with CD3-HighInt #1), XENP29475 (H2L1 CLDN18.2 ABD; 1+1 Fab-scFv-Fc with CD3-HighInt #1), XENP29476 (H1L1 CLDN18.2 ABD; 2+1 Fab₂-scFv-Fc with CD3-High), XENP29477 (H2L1 CLDN18.2 ABD; 2+1 Fab₂-scFv-Fc with CD3-High), XENP29478 (H1L1 CLDN18.2 ABD; 2+1 Fab₂-scFv-Fc with CD3-HighInt #1), and XENP29479 (H2L1 CLDN18.2 ABD; 2+1 Fab₂-scFv-Fc with CD3-HighInt #1) to SNU-601(2E4) cells. Controls used were XENP24645 (1+1 Fab-scFv-Fc with CD3-High), XENP24647 (2+1 Fab₂-scFv-Fc with CD3-High), cells only, and secondary antibody only. Consistent with the binding data, humanization preserved T cell activation by the bsAbs having humanized CLDN18.2 ABDs.

FIG. 37A-37B depicts degranulation of CD4⁺ T cells [as indicated by A) CD107a MFI on CD4⁺ T cells, and B) percentage of CD4⁺ T cells expressing CD107a] after incubation of SNU-601(2E4) for 24 hours with human PBMCs (10:1 effector to target cell ratio) and the following anti-CLDN18.2 x anti-CD3 bispecific antibodies having humanized CLDN18.2 ABD: XENP29472 (H1L1 CLDN18.2 ABD; 1+1 Fab-scFv-Fc with CD3-High), XENP29473 (H2L1 CLDN18.2 ABD; 1+1 Fab-scFv-Fc with CD3-High), XENP29474 (H1L1 CLDN18.2 ABD; 1+1 Fab-scFv-Fc with CD3-HighInt #1), XENP29475 (H2L1 CLDN18.2 ABD; 1+1 Fab-scFv-Fc with CD3-HighInt #1), XENP29476 (H1L1 CLDN18.2 ABD; 2+1 Fab₂-scFv-Fc with CD3-High), XENP29477 (H2L1 CLDN18.2 ABD; 2+1 Fab₂-scFv-Fc with CD3-High), XENP29478 (H1L1 CLDN18.2 ABD; 2+1 Fab₂-scFv-Fc with CD3-HighInt #1), and XENP29479 (H2L1 CLDN18.2 ABD; 2+1 Fab₂-scFv-Fc with CD3-HighInt #1) to SNU-601(2E4) cells. Controls used were XENP24645 (1+1 Fab-scFv-Fc with CD3-High), XENP24647 (2+1 Fab₂-scFv-Fc with CD3-High), cells only, and secondary antibody only.

FIG. 38A-38B depicts activation CD8⁺ T cells [as indicated by A) CD69 MFI on CD8⁺ T cells, and B) percentage of CD8⁺ T cells expressing CD69] after incubation of SNU-601(2E4) for 24 hours with human PBMCs (10:1 effector to target cell ratio) and the following anti-CLDN18.2 x anti-CD3 bispecific antibodies having humanized CLDN18.2 ABD: XENP29472 (H1L1 CLDN18.2 ABD; 1+1 Fab-scFv-Fc with CD3-High), XENP29473 (H2L1 CLDN18.2 ABD; 1+1 Fab-scFv-Fc with CD3-High), XENP29474 (H1L1 CLDN18.2 ABD; 1+1 Fab-scFv-Fc with CD3-HighInt #1), XENP29475 (H2L1 CLDN18.2 ABD; 1+1 Fab-scFv-Fc with CD3-HighInt #1), XENP29476 (H1L1 CLDN18.2 ABD; 2+1 Fab₂-scFv-Fc with CD3-High), XENP29477 (H2L1 CLDN18.2 ABD; 2+1 Fab₂-scFv-Fc with CD3-High), XENP29478 (H1L1 CLDN18.2 ABD; 2+1 Fab₂-scFv-Fc with CD3-HighInt #1), and XENP29479 (H2L1 CLDN18.2 ABD; 2+1 Fab₂-scFv-Fc with CD3-HighInt #1) to SNU-601(2E4) cells. Controls used were XENP24645 (1+1 Fab-scFv-Fc with CD3-High), XENP24647 (2+1 Fab₂-scFv-Fc with CD3-High), cells only, and secondary antibody only. Consistent with the binding data, humanization preserved T cell activation by the bsAbs having humanized CLDN18.2 ABDs.

FIG. 39A-39B depicts degranulation of CD8⁺ T cells [as indicated by A) CD107a MFI on CD8⁺ T cells, and B) percentage of CD8⁺ T cells expressing CD107a] after incubation of SNU-601(2E4) for 24 hours with human PBMCs (10:1 effector to target cell ratio) and the following anti-CLDN18.2 x anti-CD3 bispecific antibodies having humanized CLDN18.2 ABD: XENP29472 (H1L1 CLDN18.2 ABD; 1+1 Fab-scFv-Fc with CD3-High), XENP29473 (H2L1 CLDN18.2 ABD; 1+1 Fab-scFv-Fc with CD3-High), XENP29474 (H1L1 CLDN18.2 ABD; 1+1 Fab-scFv-Fc with CD3-HighInt #1), XENP29475 (H2L1 CLDN18.2 ABD; 1+1 Fab-scFv-Fc with CD3-HighInt #1), XENP29476 (H1L1 CLDN18.2 ABD; 2+1 Fab₂-scFv-Fc with CD3-High), XENP29477 (H2L1 CLDN18.2 ABD; 2+1 Fab₂-scFv-Fc with CD3-High), XENP29478 (H1L1 CLDN18.2 ABD; 2+1 Fab₂-scFv-Fc with CD3-HighInt #1), and XENP29479 (H2L1 CLDN18.2 ABD; 2+1 Fab₂-scFv-Fc with CD3-HighInt #1) to SNU-601(2E4) cells. Controls used were XENP24645 (1+1 Fab-scFv-Fc with CD3-High), XENP24647 (2+1 Fab₂-scFv-Fc with CD3-High), cells only, and secondary antibody only.

FIG. 40A-40B depicts A) IFNγ and B) TNFα secretion by T cells after incubation of SNU-601(2E4) for 24 hours with human PBMCs (10:1 effector to target cell ratio) and the following anti-CLDN18.2 x anti-CD3 bispecific antibodies having humanized CLDN18.2 ABD: XENP29472 (H1L1 CLDN18.2 ABD; 1+1 Fab-scFv-Fc with CD3-High), XENP29473 (H2L1 CLDN18.2 ABD; 1+1 Fab-scFv-Fc with CD3-High), XENP29474 (H1L1 CLDN18.2 ABD; 1+1 Fab-scFv-Fc with CD3-HighInt #1), XENP29475 (H2L1 CLDN18.2 ABD; 1+1 Fab-scFv-Fc with CD3-HighInt #1), XENP29476 (H1L1 CLDN18.2 ABD; 2+1 Fab₂-scFv-Fc with CD3-High), XENP29477 (H2L1 CLDN18.2 ABD; 2+1 Fab₂-scFv-Fc with CD3-High), XENP29478 (H1LI CLDN18.2 ABD; 2+1 Fab₂-scFv-Fc with CD3-HighInt #1), and XENP29479 (H2L1 CLDN18.2 ABD; 2+1 Fab₂-scFv-Fc with CD3-HighInt #1) to SNU-601(2E4) cells. Controls used were XENP24645 (1+1 Fab-scFv-Fc with CD3-High) and XENP24647 (2+1 Fab₂-scFv-Fc with CD3-High). Consistent with the binding data, humanization preserved the induction of cytokine secretion by the bsAbs having humanized CLDN18.2 ABDs.

FIG. 41A-41M depicts the sequences of several useful 2+1 Fab₂-scFv-Fc bispecific antibody format heavy chain backbones based on human IgG1, including scFvs for 6 different anti-CD3 ABDs, in both orientations, as well as sequences including and excluding the “XTEND®” FcRn variants 428L/434S (sometimes also referred to as “LS” variants) but excluding the ABDs for the other antigen. That is, Chain 1 of each set is the -CH1-hinge-CH2-CH3 sequence (the “Fab-Fc side”), to which a VH1 sequence can be added (e.g. the C-terminus of the VH1 is joined at the slash “/”) to form a full heavy chain, VH1-CH1-hinge-CH2-CH3. Chain 2 of each set is the -anti-CD3 scFv-domain linker-CH2-CH3, to which a VH1-CH1-optional domain linker- is added (e.g. the C-terminus of the domain linker is joined at the slash “/”), to form the full chain, VH1-CH1-domain linker-scFv-domain linker-CH2-CH3; in this embodiment, the scFv can be in either orientation, such that the full chain is either VH1-CH1-domain linker-VH2-scFv linker-VL2-domain linker-CH2-CH3 or VH1-CH1-domain linker-VL2-scFv linker-VH2-domain linker-CH2-CH3 (note the sequences of FIG. 41 include both options). Chain 3 is the LC domain, to which VL1 can be added to form VL1-CL. CDRs are underlined, linkers are double underlined, and domain borders are noted with a slash. It should be noted that all of the Chain 2 sequences include as the domain linker between the C-terminus of the scFv and the N-terminus of the CH2 domain the sequence GGGGSGGGGSKTHTCPPCP (SEQ ID NO:29), which is a “flexible half hinge” domain linker; however, this linker can be replaced in any FIG. 41 Chain 2 sequence with any of the “useful domain linkers” of FIG. 5, with some embodiments replacing the SEQ ID NO:29 with SEQ ID NO:28, the “full hinge C220S variant”. Each of these sequences include preferred skew, pI and ablation variants.

FIG. 42A to 42J depict several formats of the present invention. The first is the 1+1 Fab-scFv-Fc format, with a first and a second anti-antigen binding domain. Additionally, mAb-Fv, mAb-scFv, Central-scFv, Central-Fv, one armed central-scFv, one scFv-mAb, scFv-mAb and a dual scFv format are all shown. For all of the scFv domains depicted, they can be either N- to C-terminus variable heavy-(optional linker)-variable light, or the opposite. In addition, for the one armed scFv-mAb, the scFv can be attached either to the N-terminus of a heavy chain monomer or to the N-terminus of the light chain.

### DETAILED DESCRIPTION OF THE INVENTION

### A. Overview

Anti-bispecific antibodies that co-engage CD3 and a tumor antigen target are used to redirect T cells to attack and lyse targeted tumor cells. Examples include the BiTE® and DART formats, which monovalently engage CD3 and a tumor antigen. While the CD3-targeting approach has shown considerable promise, a common side effect of such therapies is the associated production of cytokines, often leading to toxic cytokine release syndrome. Because the anti-CD3 binding domain of the bispecific antibody engages all T cells, the high cytokine-producing CD4 T cell subset is recruited. Moreover, the CD4 T cell subset includes regulatory T cells, whose recruitment and expansion can potentially lead to immune suppression and have a negative impact on long-term tumor suppression. In addition, these formats do not contain Fc domains and show very short serum half-lives in patients.

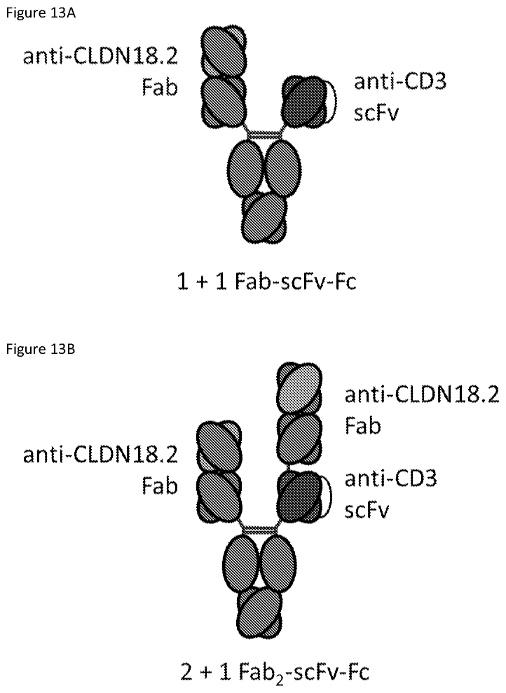

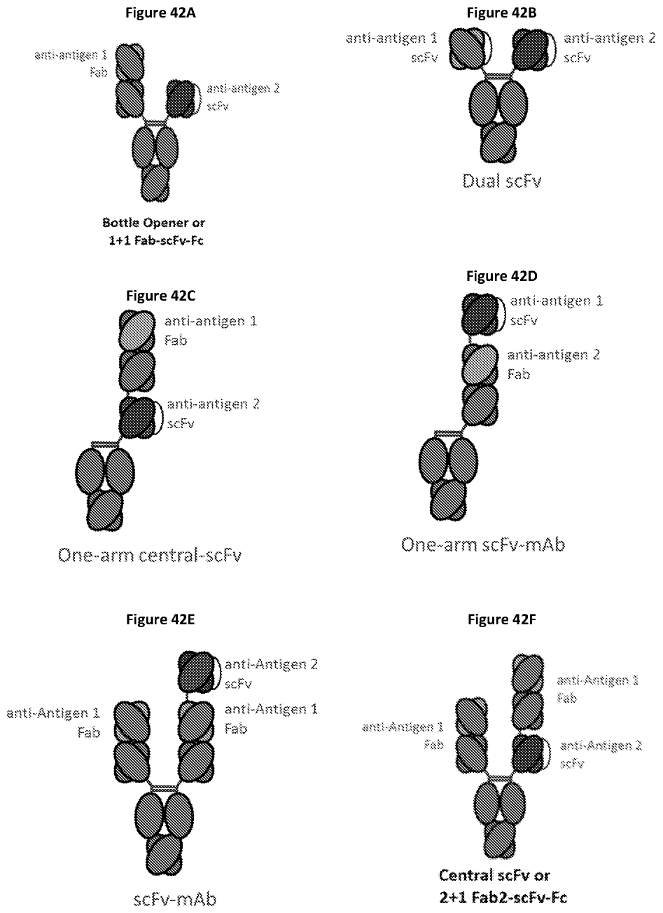

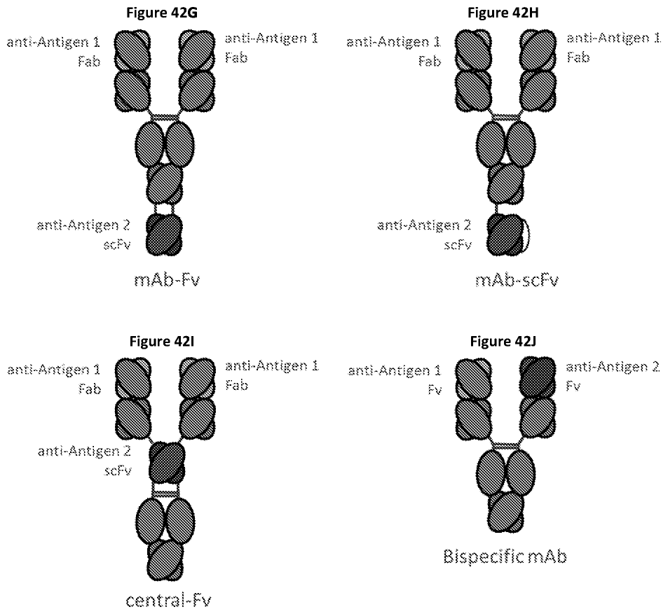

In particular, provided herein are anti-CD3, anti-CLDN18.2 bispecific antibodies in a variety of Formats such as those depicted in FIGS. 13 and 42. These bispecific antibodies are useful for the treatment of cancers, particularly those such as gastric, esophageal, and pancreatic cancers. Such antibodies are used to direct CD3+ effector T cells to CLDN18.2+ tumors, thereby allowing the CD3+ effector T cells to attack and lyse the CLDN18.2+ tumors.

Additionally, the invention provides bispecific antibodies that have different binding affinities to human CD3 that can alter or reduce the potential side effects of anti-CD3 therapy. That is, in some embodiments the present invention provides antibody constructs comprising anti-CD3 antigen binding domains that are “strong” or “high affinity” binders to CD3 (e.g. one example are heavy and light variable domains depicted as H1.30_L1.47 (optionally including a charged linker as appropriate)) and also bind to CLDN18.2. In other embodiments, the present invention provides antibody constructs comprising anti-CD3 antigen binding domains that are “lite” or “lower affinity” binders to CD3. Additional embodiments provides antibody constructs comprising anti-CD3 antigen binding domains that have intermediate or “medium” affinity to CD3 that also bind to CD38. While a very large number of anti-CD3 antigen binding domains (ABDs) can be used, particularly useful embodiments use 6 different anti-CD3 ABDs, although they can be used in two scFv orientations as discussed herein. Affinity is generally measured using a Biacore assay.

It should be appreciated that the “high, medium, low” anti-CD3 sequences of the present invention can be used in a variety of heterodimerization formats as depicted in Figures. In general, due to the potential side effects of T cell recruitment, preferred embodiments utilize formats that only bind CD3 monovalently, such as depicted in FIGS. 13A and 13B, and in the formats depicted herein, it is the CD3 ABD that is a scFv as more fully described herein. In contrast, the bispecific antibodies of the invention can bind CLDN18.2 either monovalently (e.g. FIG. 13A) or bivalently (e.g. FIG. 13B).

Accordingly, in one aspect, provided herein are heterodimeric antibodies that bind to two different antigens, e.g. the antibodies are “bispecific”, in that they bind two different target antigens, e.g. CD3 and CLDN18.2 as described herein. These heterodimeric antibodies can bind these target antigens either monovalently (e.g. there is a single antigen binding domain such as a variable heavy and variable light domain pair) or bivalently (there are two antigen binding domains that each independently bind the antigen). The heterodimeric antibodies provided herein are based on the use different monomers which contain amino acid substitutions that “skew” formation of heterodimers over homodimers, as is more fully outlined below, coupled with “pI variants” that allow simple purification of the heterodimers away from the homodimers, as is similarly outlined below. The heterodimeric bispecific antibodies provided generally rely on the use of engineered or variant Fc domains that can self-assemble in production cells to produce heterodimeric proteins, and methods to generate and purify such heterodimeric proteins.

### C. Nomenclature

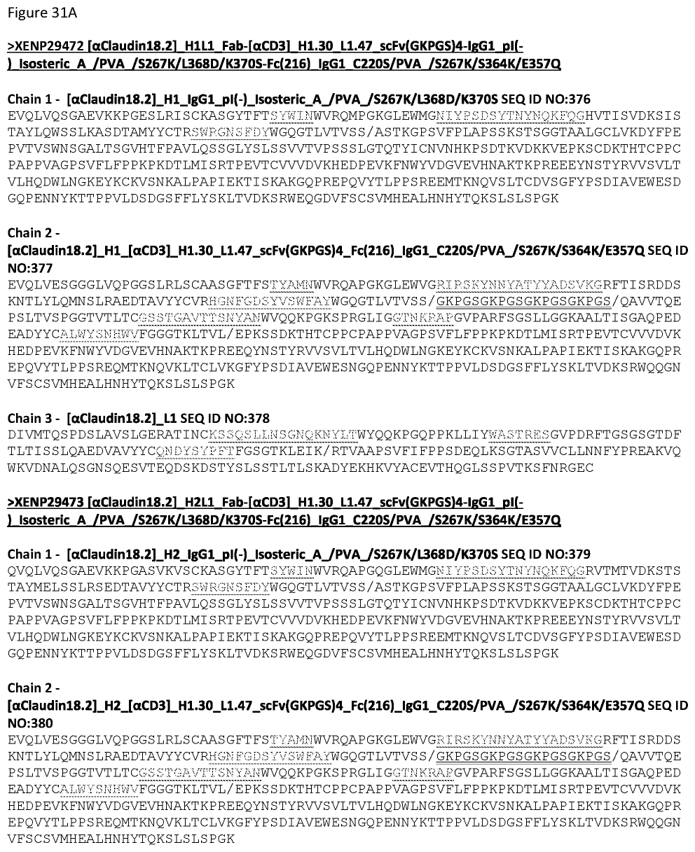

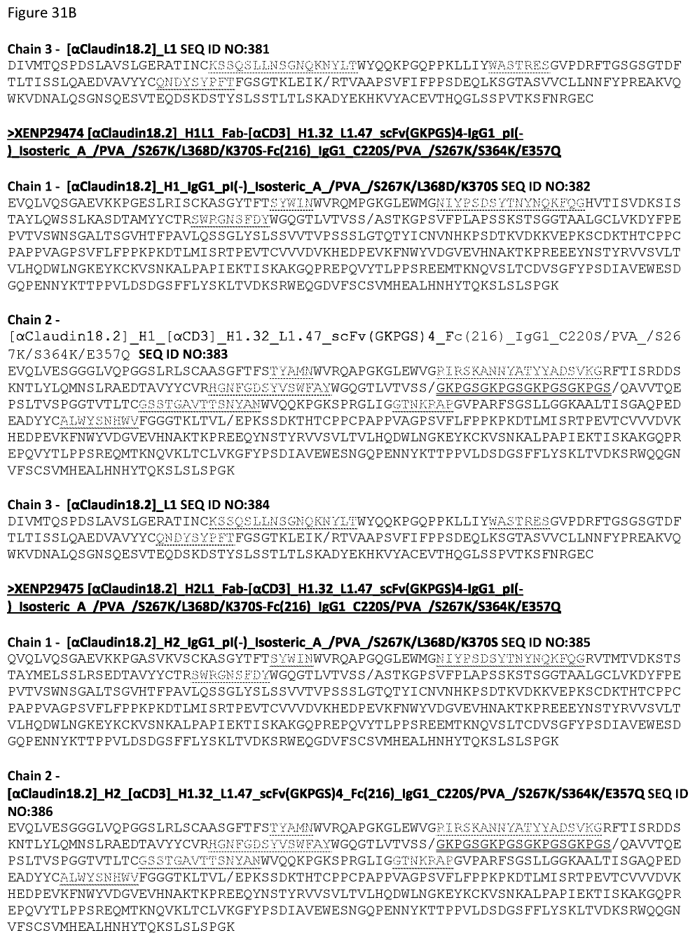

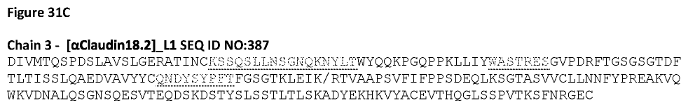

The bispecific antibodies of the invention are listed in several different formats. Each polypeptide is given a unique “XENP” number, although as will be appreciated in the art, a longer sequence might contain a shorter one. For example, the heavy chain of the scFv side monomer of a 1+1 Fab-scFv-Fc format for a given sequence will have a first XENP number, while the scFv domain could have a different XENP number. Some molecules have three polypeptides, so the XENP number, with the components, is used as a name. Thus, the molecule XENP29472, which is in bottle opener format, comprises three sequences: XENP29472 Chain 1, XENP29472 Chain 2 and XENP29472 Chain 3 (FIG. 31A). These XENP numbers are in the sequence listing as well as identifiers, and used in the Figures. In addition, one molecule, comprising the three components, gives rise to multiple sequence identifiers. For example, the listing of the Fab monomer has the full length sequence, the variable heavy sequence and the three CDRs of the variable heavy sequence; the light chain has a full length sequence, a variable light sequence and the three CDRs of the variable light sequence; and the scFv-Fc domain has a full length sequence, an scFv sequence, a variable light sequence, 3 light CDRs, a scFv linker, a variable heavy sequence and 3 heavy CDRs; note that all molecules herein with a scFv domain use a single charged scFv linker (+H), although others can be used. In addition, the naming nomenclature of particular variable domains uses a “Hx.xx_Ly.yy” type of format, with the numbers being unique identifiers to particular variable chain sequences. Thus, the variable domain of the Fab side of XENP29472 is “H1L1”, which indicates that the variable heavy domain, H1, was combined with the light domain L1. In the case that these sequences are used as scFvs, the designation “H1L1”, indicates that the variable heavy domain, H1, was combined with the light domain, L1, and is in VH-linker-VL orientation, from N- to C-terminus. This molecule with the identical sequences of the heavy and light variable domains but in the reverse order would be named “L1H1”. Similarly, different constructs may “mix and match” the heavy and light chains as will be evident from the sequence listing and the Figures.

### D. Definitions

In order that the application may be more completely understood, several definitions are set forth below. Such definitions are meant to encompass grammatical equivalents.

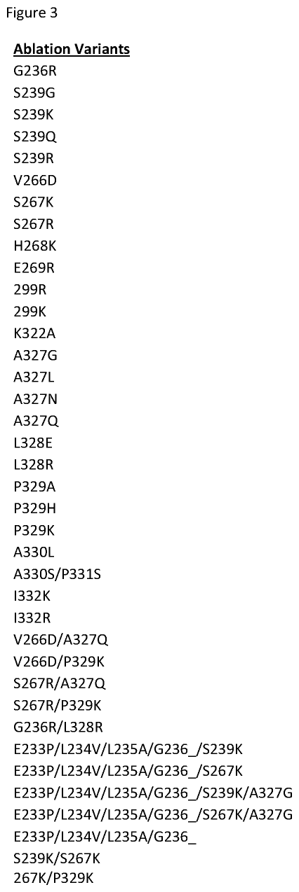

By “ablation” herein is meant a decrease or removal of activity. Thus for example, “ablating FcγR binding” means the Fc region amino acid variant has less than 50% starting binding as compared to an Fc region not containing the specific variant, with more than 70-80-90-95-98% loss of activity being preferred, and in general, with the activity being below the level of detectable binding in a Biacore, SPR or BLI assay. Of particular use in the ablation of FcγR binding are those shown in FIG. 3, which generally are added to both monomers.

By “ADCC” or “antibody dependent cell-mediated cytotoxicity” as used herein is meant the cell-mediated reaction wherein nonspecific cytotoxic cells that express FcγRs recognize bound antibody on a target cell and subsequently cause lysis of the target cell. ADCC is correlated with binding to FcγRIIIa; increased binding to FcγRIIIa leads to an increase in ADCC activity.

By “ADCP” or antibody dependent cell-mediated phagocytosis as used herein is meant the cell-mediated reaction wherein nonspecific phagocytic cells that express FcγRs recognize bound antibody on a target cell and subsequently cause phagocytosis of the target cell.

By “antigen binding domain” or “ABD” herein is meant a set of six Complementary Determining Regions (CDRs) that, when present as part of a polypeptide sequence, specifically binds a target antigen as discussed herein. Thus, a “checkpoint antigen binding domain” binds a target checkpoint antigen as outlined herein. As is known in the art, these CDRs are generally present as a first set of variable heavy CDRs (VHCDRs or VHCDRs) and a second set of variable light CDRs (VLCDRs or VLCDRs), each comprising three CDRs: VHCDR1, VHCDR2, VHCDR3 for the heavy chain and VLCDR1, VLCDR2 and VLCDR3 for the light. The CDRs are present in the variable heavy and variable light domains, respectively, and together form an Fv region. (See Table 1 and related discussion above for CDR numbering schemes). Thus, in some cases, the six CDRs of the antigen binding domain are contributed by a variable heavy and a variable light domain. In a “Fab” format, the set of 6 CDRs are contributed by two different polypeptide sequences, the variable heavy domain (VH or VH; containing the VHCDR1, VHCDR2 and VHCDR3) and the variable light domain (VL or VL; containing the VLCDR1, VLCDR2 and VLCDR3), with the C-terminus of the VH domain being attached to the N-terminus of the CH1 domain of the heavy chain and the C-terminus of the VL domain being attached to the N-terminus of the constant light domain (and thus forming the light chain). In a scFv format, the VH and VL domains are covalently attached, generally through the use of a linker (a “scFv linker”) as outlined herein, into a single polypeptide sequence, which can be either (starting from the N-terminus) VH-linker-VL or VL-linker-VH, with the former being generally preferred (including optional domain linkers on each side, depending on the format used (e.g. from FIG. 13). In general, the C-terminus of the scFv domain is attached to the N-terminus of the hinge in the second monomer.

By “modification” herein is meant an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always to an amino acid coded for by DNA, e.g. the 20 amino acids that have codons in DNA and RNA.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution E272Y refers to a variant polypeptide, in this case an Fc variant, in which the glutamic acid at position 272 is replaced with tyrosine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not an amino acid substitution.

By “amino acid insertion” or “insertion” as used herein is meant the addition of an amino acid sequence at a particular position in a parent polypeptide sequence. For example, −233E or 233E designates an insertion of glutamic acid after position 233 and before position 234. Additionally, −233ADE or A233ADE designates an insertion of AlaAspGlu after position 233 and before position 234.

By “amino acid deletion” or “deletion” as used herein is meant the removal of an amino acid sequence at a particular position in a parent polypeptide sequence. For example, E233- or E233 #, E233( ) or E233del designates a deletion of glutamic acid at position 233. Additionally, EDA233- or EDA233 # designates a deletion of the sequence GluAspAla that begins at position 233.

By “variant protein” or “protein variant”, or “variant” as used herein is meant a protein that differs from that of a parent protein by virtue of at least one amino acid modification. The protein variant has at least one amino acid modification compared to the parent protein, yet not so many that the variant protein will not align with the parental protein using an alignment program such as that described below. In general, variant proteins (such as variant Fc domains, etc., outlined herein, are generally at least 75, 80, 85, 90, 91, 92, 93, 94, 95, 96, 97, 98 or 99% identical to the parent protein, using the alignment programs described below, such as BLAST.

As described below, in some embodiments the parent polypeptide, for example an Fc parent polypeptide, is a human wild-type sequence, such as the heavy constant domain or Fc region from IgG1, IgG2, IgG3 or IgG4, although human sequences with variants can also serve as “parent polypeptides”, for example the IgG½ hybrid of US Publication 2006/0134105 can be included. The protein variant sequence herein will preferably possess at least about 80% identity with a parent protein sequence, and most preferably at least about 90% identity, more preferably at least about 95-98-99% identity. Accordingly, by “antibody variant” or “variant antibody” as used herein is meant an antibody that differs from a parent antibody by virtue of at least one amino acid modification, “IgG variant” or “variant IgG” as used herein is meant an antibody that differs from a parent IgG (again, in many cases, from a human IgG sequence) by virtue of at least one amino acid modification, and “immunoglobulin variant” or “variant immunoglobulin” as used herein is meant an immunoglobulin sequence that differs from that of a parent immunoglobulin sequence by virtue of at least one amino acid modification. “Fc variant” or “variant Fc” as used herein is meant a protein comprising an amino acid modification in an Fc domain as compared to an Fc domain of human IgG1, IgG2 or IgG4.

The Fc variants of the present invention are defined according to the amino acid modifications that compose them. Thus, for example, N434S or 434S is an Fc variant with the substitution serine at position 434 relative to the parent Fc polypeptide, wherein the numbering is according to the EU index. Likewise, M428L/N434S defines an Fc variant with the substitutions M428L and N434S relative to the parent Fc polypeptide. The identity of the WT amino acid may be unspecified, in which case the aforementioned variant is referred to as 428L/434S. It is noted that the order in which substitutions are provided is arbitrary, that is to say that, for example, N434S/M428L is the same Fc variant as M428L/N434S, and so on. For all positions discussed in the present invention that relate to antibodies, unless otherwise noted, amino acid position numbering is according to the EU index. The EU index or EU index as in Kabat or EU numbering scheme refers to the numbering of the EU antibody. Kabat et al. collected numerous primary sequences of the variable regions of heavy chains and light chains. Based on the degree of conservation of the sequences, they classified individual primary sequences into the CDR and the framework and made a list thereof (see SEQUENCES OF IMMUNOLOGICAL INTEREST, 5th edition, NIH publication, No. 91-3242, E. A. Kabat et al., entirely incorporated by reference). See also Edelman et al., 1969, Proc Natl Acad Sci USA 63:78-85, hereby entirely incorporated by reference. The modification can be an addition, deletion, or substitution.

By “protein” herein is meant at least two covalently attached amino acids, which includes proteins, polypeptides, oligopeptides and peptides. In addition, polypeptides that make up the antibodies of the invention may include synthetic derivatization of one or more side chains or termini, glycosylation, PEGylation, circular permutation, cyclization, linkers to other molecules, fusion to proteins or protein domains, and addition of peptide tags or labels.

By “residue” as used herein is meant a position in a protein and its associated amino acid identity. For example, Asparagine 297 (also referred to as Asn297 or N297) is a residue at position 297 in the human antibody IgG1.

By “Fab” or “Fab region” as used herein is meant the polypeptide that comprises the VH, CH1, VL, and CL immunoglobulin domains, generally on two different polypeptide chains (e.g. VH-CH1 on one chain and VL-CL on the other). Fab may refer to this region in isolation, or this region in the context of a bispecific antibody of the invention. In the context of a Fab, the Fab comprises an Fv region in addition to the CH1 and CL domains.

By “Fv” or “Fv fragment” or “Fv region” as used herein is meant a polypeptide that comprises the VL and VH domains of an ABD. Fv regions can be formatted as both Fabs (as discussed above, generally two different polypeptides that also include the constant regions as outlined above) and scFvs, where the VL and VH domains are combined (generally with a linker as discussed herein) to form an scFv.

By “single chain Fv” or “scFv” herein is meant a variable heavy domain covalently attached to a variable light domain, generally using a scFv linker as discussed herein, to form a scFv or scFv domain. A scFv domain can be in either orientation from N- to C-terminus (VH-linker-VL or VL-linker-VH). In the sequences depicted in the sequence listing and in the figures, the order of the VH and VL domain is indicated in the name, e.g. H.X_L.Y means N- to C-terminal is VH-linker-VL, and L. Y_H.X is VL-linker-VH.

By “IgG subclass modification” or “isotype modification” as used herein is meant an amino acid modification that converts one amino acid of one IgG isotype to the corresponding amino acid in a different, aligned IgG isotype. For example, because IgG1 comprises a tyrosine and IgG2 a phenylalanine at EU position 296, a F296Y substitution in IgG2 is considered an IgG subclass modification.

By “non-naturally occurring modification” as used herein is meant an amino acid modification that is not isotypic. For example, because none of the human IgGs comprise a serine at position 434, the substitution 434S in IgG1, IgG2, IgG3, or IgG4 (or hybrids thereof) is considered a non-naturally occurring modification.

By “amino acid” and “amino acid identity” as used herein is meant one of the 20 naturally occurring amino acids that are coded for by DNA and RNA.

By “effector function” as used herein is meant a biochemical event that results from the interaction of an antibody Fc region with an Fc receptor or ligand. Effector functions include but are not limited to ADCC, ADCP, and CDC.

By “IgG Fc ligand” as used herein is meant a molecule, preferably a polypeptide, from any organism that binds to the Fc region of an IgG antibody to form an Fc/Fc ligand complex. Fc ligands include but are not limited to FcγRIs, FcγRIIs, FcγRIIIs, FcRn, C1q, C3, mannan binding lectin, mannose receptor, staphylococcal protein A, streptococcal protein G, and viral FcγR. Fc ligands also include Fc receptor homologs (FcRH), which are a family of Fc receptors that are homologous to the FcγRs (Davis et al., 2002, Immunological Reviews 190:123-136, entirely incorporated by reference). Fc ligands may include undiscovered molecules that bind Fc. Particular IgG Fc ligands are FcRn and Fc gamma receptors. By “Fc ligand” as used herein is meant a molecule, preferably a polypeptide, from any organism that binds to the Fc region of an antibody to form an Fc/Fc ligand complex.

By “Fc gamma receptor”, “FcγR” or “FcgammaR” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIb-NA1 and FcγRIIb-NA2) (Jefferis et al., 2002, Immunol Lett 82:57-65, entirely incorporated by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII (CD16), and FcγRIII-2 (CD16-2), as well as any undiscovered mouse FcγRs or FcγR isoforms or allotypes.

By “FcRn” or “neonatal Fc Receptor” as used herein is meant a protein that binds the IgG antibody Fc region and is encoded at least in part by an FcRn gene. The FcRn may be from any organism, including but not limited to humans, mice, rats, rabbits, and monkeys. As is known in the art, the functional FcRn protein comprises two polypeptides, often referred to as the heavy chain and light chain. The light chain is beta-2-microglobulin and the heavy chain is encoded by the FcRn gene. Unless otherwise noted herein, FcRn or an FcRn protein refers to the complex of FcRn heavy chain with beta-2-microglobulin. A variety of FcRn variants used to increase binding to the FcRn receptor, and in some cases, to increase serum half-life. An “FcRn variant” is one that increases binding to the FcRn receptor, and suitable FcRn variants are shown below.

By “parent polypeptide” as used herein is meant a starting polypeptide that is subsequently modified to generate a variant. The parent polypeptide may be a naturally occurring polypeptide, or a variant or engineered version of a naturally occurring polypeptide. Accordingly, by “parent immunoglobulin” as used herein is meant an unmodified immunoglobulin polypeptide that is modified to generate a variant, and by “parent antibody” as used herein is meant an unmodified antibody that is modified to generate a variant antibody. It should be noted that “parent antibody” includes known commercial, recombinantly produced antibodies as outlined below. In this context, a “parent Fc domain” will be relative to the recited variant; thus, a “variant human IgG1 Fc domain” is compared to the parent Fc domain of human IgG1, a “variant human IgG4 Fc domain” is compared to the parent Fc domain human IgG4, etc.

By “Fc” or “Fc region” or “Fc domain” as used herein is meant the polypeptide comprising the CH2-CH3 domains of an IgG molecule, and in some cases, inclusive of the hinge. In EU numbering for human IgG1, the CH2-CH3 domain comprises amino acids 231 to 447, and the hinge is 216 to 230. Thus the definition of “Fc domain” includes both amino acids 231-447 (CH2-CH3) or 216-447 (hinge-CH2-CH3), or fragments thereof. An “Fc fragment” in this context may contain fewer amino acids from either or both of the N- and C-termini but still retains the ability to form a dimer with another Fc domain or Fc fragment as can be detected using standard methods, generally based on size (e.g. non-denaturing chromatography, size exclusion chromatography, etc.) Human IgG Fc domains are of particular use in the present invention, and can be the Fc domain from human IgG1, IgG2 or IgG4.

A “variant Fc domain” contains amino acid modifications as compared to a parental Fc domain. Thus, a “variant human IgG1 Fc domain” is one that contains amino acid modifications (generally amino acid substitutions, although in the case of ablation variants, amino acid deletions are included) as compared to the human IgG1 Fc domain. In general, variant Fc domains have at least about 80, 85, 90, 95, 97, 98 or 99 percent identity to the corresponding parental human IgG Fc domain (using the identity algorithms discussed below, with one embodiment utilizing the BLAST algorithm as is known in the art, using default parameters). Alternatively, the variant Fc domains can have from 1, 2, 3, 4, 5, 6, 7, 8, 9, 10, 11,12, 13, 14, 15, 16, 11, 12, 13, 14, 15, 16, 17, 18, 19 or 20 amino acid modifications as compared to the parental Fc domain. Alternatively, the variant Fc domains can have up to 1, 2, 3, 4, 5, 6, 7, 8, 9, 10, 11, 12, 13, 14, 15, 16, 11, 12, 13, 14, 15, 16, 17, 18, 19 or 20 amino acid modifications as compared to the parental Fc domain. Additionally, as discussed herein, the variant Fc domains herein still retain the ability to form a dimer with another Fc domain as measured using known techniques as described herein, such as non-denaturing gel electrophoresis.

By “heavy chain constant region” herein is meant the CH1-hinge-CH2-CH3 portion of an antibody (or fragments thereof), excluding the variable heavy domain; in EU numbering of human IgG1 this is amino acids 118-447 By “heavy chain constant region fragment” herein is meant a heavy chain constant region that contains fewer amino acids from either or both of the N- and C-termini but still retains the ability to form a dimer with another heavy chain constant region.

By “position” as used herein is meant a location in the sequence of a protein. Positions may be numbered sequentially, or according to an established format, for example the EU index for antibody numbering.

By “target antigen” as used herein is meant the molecule that is bound specifically by the antigen binding domain comprising the variable regions of a given antibody. As discussed below, in the present case the target antigens are checkpoint inhibitor proteins.

By “strandedness” in the context of the monomers of the heterodimeric antibodies of the invention herein is meant that, similar to the two strands of DNA that “match”, heterodimerization variants are incorporated into each monomer so as to preserve the ability to “match” to form heterodimers. For example, if some pI variants are engineered into monomer A (e.g. making the pI higher) then steric variants that are “charge pairs” that can be utilized as well do not interfere with the pI variants, e.g. the charge variants that make a pI higher are put on the same “strand” or “monomer” to preserve both functionalities. Similarly, for “skew” variants that come in pairs of a set as more fully outlined below, the skilled artisan will consider pI in deciding into which strand or monomer one set of the pair will go, such that pI separation is maximized using the pI of the skews as well.

By “target cell” as used herein is meant a cell that expresses a target antigen.

By “host cell” in the context of producing a bispecific antibody according to the invention herein is meant a cell that contains the exogeneous nucleic acids encoding the components of the bispecific antibody and is capable of expressing the bispecific antibody under suitable conditions. Suitable host cells are discussed below.

By “variable region” or “variable domain” as used herein is meant the region of an immunoglobulin that comprises one or more Ig domains substantially encoded by any of the Vκ, Vλ, and/or VH genes that make up the kappa, lambda, and heavy chain immunoglobulin genetic loci respectively, and contains the CDRs that confer antigen specificity. Thus, a “variable heavy domain” pairs with a “variable light domain” to form an antigen binding domain (“ABD”). In addition, each variable domain comprises three hypervariable regions (“complementary determining regions,” “CDRs”) (VHCDR1, VHCDR2 and VHCDR3 for the variable heavy domain and VLCDR1, VLCDR2 and VLCDR3 for the variable light domain) and four framework (FR) regions, arranged from amino-terminus to carboxy-terminus in the following order: FR1-CDR1-FR2-CDR2-FR3-CDR3-FR4.

By “wild type or WT” herein is meant an amino acid sequence or a nucleotide sequence that is found in nature, including allelic variations. A WT protein has an amino acid sequence or a nucleotide sequence that has not been intentionally modified.

The invention provides a number of antibody domains that have sequence identity to human antibody domains. Sequence identity between two similar sequences (e.g., antibody variable domains) can be measured by algorithms such as that of Smith, T. F. & Waterman, M. S. (1981) “Comparison Of Biosequences,” Adv. Appl. Math. 2:482 [local homology algorithm]; Needleman, S. B. & Wunsch, C D. (1970) “A General Method Applicable To The Search For Similarities In The Amino Acid Sequence Of Two Proteins,” J. Mol. Biol.48:443 [homology alignment algorithm], Pearson, W. R. & Lipman, D. J. (1988) “Improved Tools For Biological Sequence Comparison,” Proc. Natl. Acad. Sci. (U.S.A.) 85:2444 [search for similarity method]; or Altschul, S. F. et al, (1990) “Basic Local Alignment Search Tool,” J. Mol. Biol. 215:403-10, the “BLAST” algorithm, see https://blast.ncbi.nlm.nih.gov/Blast.cgi. When using any of the aforementioned algorithms, the default parameters (for Window length, gap penalty, etc.) are used. In one embodiment, sequence identity is done using the BLAST algorithm, using default parameters

The antibodies of the present invention are generally isolated or recombinant. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated antibody,” refers to an antibody which is substantially free of other antibodies having different antigenic specificities. “Recombinant” means the antibodies are generated using recombinant nucleic acid techniques in exogeneous host cells, and they can be isolated as well.

“Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a KD for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction. Binding affinity is generally measured using a Biacore, SPR or BLI assay.

### E. Antibodies

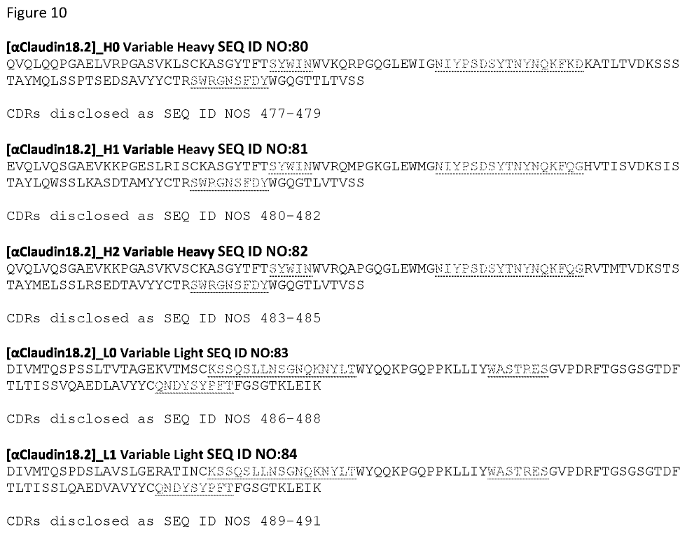

In one aspect, provided herein are bispecific antibodies that bind to CLDN18.2 and CD3, in various formats as outlined below, and generally depicted in FIGS. 13 and 42. These bispecific, heterodimeric antibodies include a CLDN18.2 binding domain. In certain embodiments, the CLDN18.2 binding domain includes the VHCDR1, VHCDR2, VHCDR3, VLCDR1, VLCDR2 and VLCDR3 sequences of an CLDN18.2 binding domain selected from the group consisting of those depicted in FIG. 10. In some embodiments, the CLDN18.2 binding domain includes the underlined VHCDR1, VHCDR2, VHCDR3, VLCDR1, VLCDR2 and VLCDR3 sequences of an CLDN18.2 binding domain selected from those depicted in FIG. 10.

These bispecific heterodimeric antibodies bind CLDN18.2 and CD3. Such antibodies include a CD3 binding domain and at least one CLDN18.2 binding domain. Any suitable CLDN18.2 binding domain can be included in the anti-CLDN18.2 X anti-CD3 bispecific antibody. In some embodiments, the anti-CLDN18.2 X anti-CD3 bispecific antibody includes one, two, three, four or more CLDN18.2 binding domains, including but not limited to those depicted in FIG. 10. In certain embodiments, the anti-CLDN18.2 X anti-CD3 antibody includes a CLDN18.2 binding domain that includes the VHCDR1, VHCDR2, VHCDR3, VLCDR1, VLCDR2 and VLCDR3 sequences of an CLDN18.2 binding domain selected from the group consisting of those depicted in FIG. 10. In some embodiments, the anti-CLDN18.2 X anti-CD3 antibody includes a CLDN18.2 binding domain that includes the underlined VHCDR1, VHCDR2, VHCDR3, VLCDR1, VLCDR2 and VLCDR3 sequences of an CLDN18.2 binding domain selected from the group consisting of those depicted in FIG. 10. In some embodiments, the anti-CLDN18.2 X anti-CD3 antibody includes a CLDN18.2 binding domain that includes the variable heavy domain and variable light domain of an CLDN18.2 binding domain selected from the group consisting of those depicted in FIG. 10. In an exemplary embodiment, the anti-CLDN18.2 X anti-CD3 antibody includes an anti-CLDN18.2 H1L1 or an H2L1 binding domain.

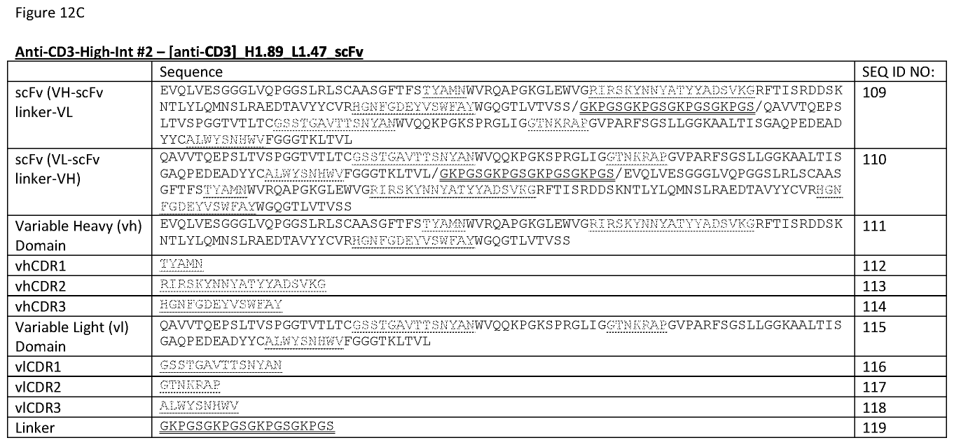

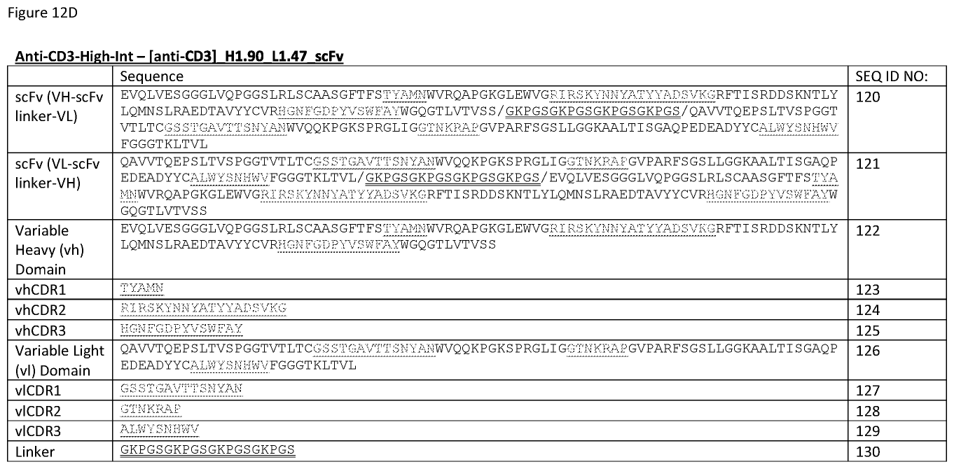

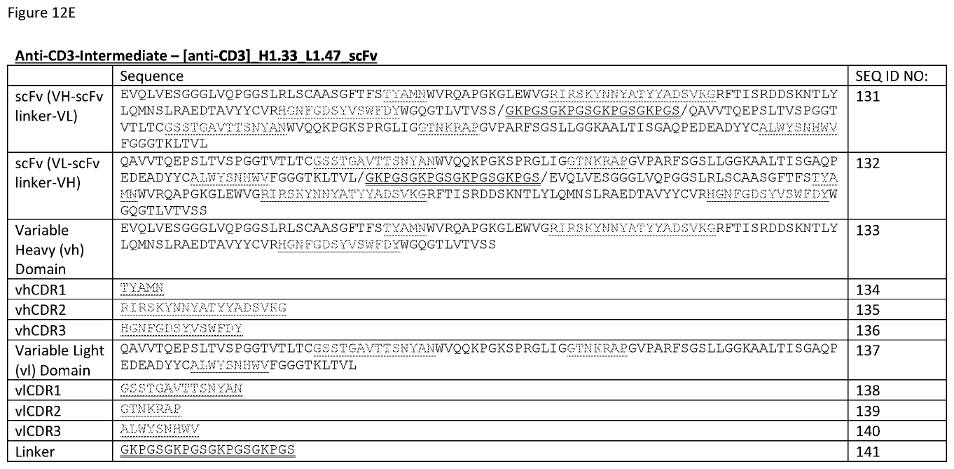

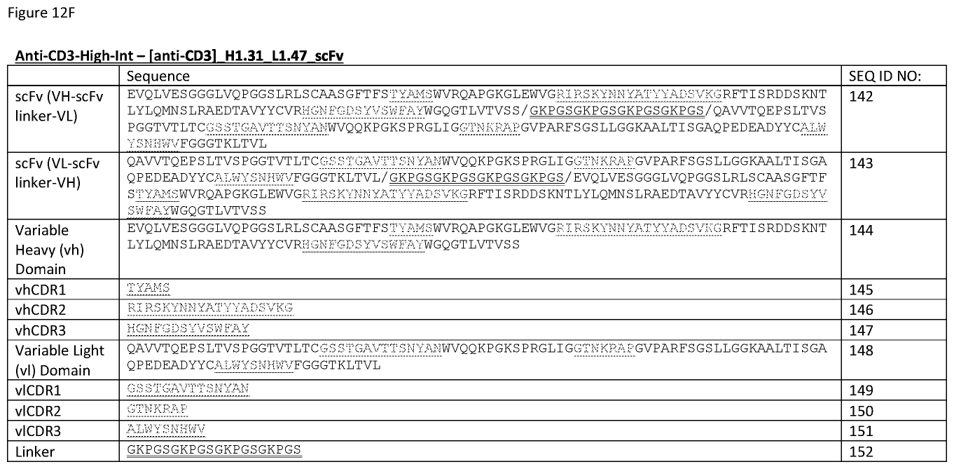

The anti-CLDN18.2 x anti-CD3 antibody provided herein can include any suitable CD3 binding domain. In certain embodiments, the anti-CLDN18.2 X anti-CD3 antibody includes a CD3 binding domain that includes the VHCDR1, VHCDR2, VHCDR3, VLCDR1, VLCDR2 and VLCDR3 sequences of a CD3 binding domain selected from the group consisting of those depicted in FIGS. 12. In some embodiments, the anti-CLDN18.2 X anti-CD3 antibody includes a CD3 binding domain that includes the underlined VHCDR1, VHCDR2, VHCDR3, VLCDR1, VLCDR2 and VLCDR3 sequences of a CD3 binding domain selected from the group consisting of those depicted in FIG. 12. In some embodiments, the anti-CLDN18.2 X anti-CD3 antibody includes a CD3 binding domain that includes the variable heavy domain and variable light domain of a CD3 binding domain selected from the group consisting of those depicted in FIG. 12. In some embodiments, the CD3 binding domain is selected from anti-CD3 H1.30_L1.47, anti-CD3 H1.32_L1.47; anti-CD3 H1.89_L1.48; anti-CD3 H1.90_L1.47; Anti-CD3 H1.33_L1.47; and anti-CD3 H1.31_L1.47. As outlined herein, these anti-CD3 antigen binding domains (CD3-ABDs) can be used in scFv formats in either orientation (e.g. from N- to C-terminal, VH-scFv linker-VL or VL-scFv linker-VH).

As used herein, term “antibody” is used generally. Antibodies that find use in the present invention can take on a number of formats as described herein, including traditional antibodies as well as antibody derivatives, fragments and mimetics, described herein.

Traditional antibody structural units typically comprise a tetramer. Each tetramer is typically composed of two identical pairs of polypeptide chains, each pair having one “light” (typically having a molecular weight of about 25 kDa) and one “heavy” chain (typically having a molecular weight of about 50-70 kDa). Human light chains are classified as kappa and lambda light chains. The present invention is directed to the IgG class, which has several subclasses, including, but not limited to IgG1, IgG2, IgG3, and IgG4. It should be noted that IgG1 has different allotypes with polymorphisms at 356 (D or E) and 358 (L or M). The sequences depicted herein use the 356D/358M allotype, however the other allotype is included herein. That is, any sequence inclusive of an IgG1 Fc domain included herein can have 356E/358L replacing the 356D/358M allotype.

In addition, many of the antibodies herein have at least one the cysteines at position 220 replaced by a serine; generally this is the on the “scFv monomer” side for most of the sequences depicted herein, although it can also be on the “Fab monomer” side, or both, to reduce disulfide formation. Specifically included within the sequences herein are one or both of these cysteines replaced (C220S).

Thus, “isotype” as used herein is meant any of the subclasses of immunoglobulins defined by the chemical and antigenic characteristics of their constant regions. It should be understood that therapeutic antibodies can also comprise hybrids of isotypes and/or subclasses. For example, as shown in US Publication 2009/0163699, incorporated by reference, the present invention includes the use of human IgG1/G2 hybrids.

The hypervariable region generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region; Kabat et al., SEQUENCES OF PROTEINS OF IMMUNOLOGICAL INTEREST, 5th Ed. Public Health Service, National Institutes of Health, Bethesda, Md. (1991) and/or those residues forming a hypervariable loop (e.g. residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917. Specific CDRs of the invention are described below.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the VHCDRs (e.g. VHCDR1, VHCDR2 and VHCDR3) and the disclosure of each variable light region is a disclosure of the VLCDRs (e.g. VLCDR1, VLCDR2 and VLCDR3). A useful comparison of CDR numbering is as below, see Lafranc et al., Dev. Comp. Immunol. 27(1):55-77 (2003):

TABLE 1

| | Kabat + | | | | | | |
|---|---|---|---|---|---|---|---|
| | Chothia | IMGT | Kabat | AbM | Chothia | Contact | Xencor |
| VHCDR1 | 26-35 | 27-38 | 31-35 | 26-35 | 26-32 | 30-35 | 27-35 |
| VHCDR2 | 50-65 | 56-65 | 50-65 | 50-58 | 52-56 | 47-58 | 54-61 |
| VHCDR3 | 95-102 | 105-117 | 95-102 | 95-102 | 95-102 | 93-101 | 103-116 |
| VLCDR1 | 24-34 | 27-38 | 24-34 | 24-34 | 24-34 | 30-36 | 27-38 |
| VLCDR2 | 50-56 | 56-65 | 50-56 | 50-56 | 50-56 | 46-55 | 56-62 |
| VLCDR3 | 89-97 | 105-117 | 89-97 | 89-97 | 89-97 | 89-96 | 97-105 |

Throughout the present specification, the Kabat numbering system is generally used when referring to a residue in the variable domain (approximately, residues 1-107 of the light chain variable region and residues 1-113 of the heavy chain variable region) and the EU numbering system for Fc regions (e.g., Kabat et al., supra (1991)).

Another type of Ig domain of the heavy chain is the hinge region. By “hinge” or “hinge region” or “antibody hinge region” or “hinge domain” herein is meant the flexible polypeptide comprising the amino acids between the first and second constant domains of an antibody. Structurally, the IgG CH1 domain ends at EU position 215, and the IgG CH2 domain begins at residue EU position 231. Thus for IgG the antibody hinge is herein defined to include positions 216 (E216 in IgG1) to 230 (p230 in IgG1), wherein the numbering is according to the EU index as in Kabat. In some cases, a “hinge fragment” is used, which contains fewer amino acids at either or both of the N- and C-termini of the hinge domain. As discussed below, sometimes the hinge is a domain linker; in these embodiments, useful domain linkers based on the hinge are shown in FIG. 5, and in particular, SEQ ID NO:28 can find particular use in the “1+1 Fab-scFv-Fc”, as well as in the “2+1 Fab₂-scFv-Fc” format. As noted herein, pI variants can be made in the hinge region as well.

The light chain generally comprises two domains, the variable light domain (containing the light chain CDRs and together with the variable heavy domains forming the Fv region), and a constant light chain region (often referred to as CL or Cκ).

Another region of interest for additional substitutions, outlined below, is the Fc region.

The present invention provides a large number of different CDR sets. In this case, a “full CDR set” comprises the three variable light and three variable heavy CDRs, e.g. a VLCDR1, VLCDR2, VLCDR3, VHCDR1, VHCDR2 and VHCDR3. These can be part of a larger variable light or variable heavy domain, respectfully. In addition, as more fully outlined herein, the variable heavy and variable light domains can be on separate polypeptide chains, when a heavy and light chain is used (for example when Fabs are used), or on a single polypeptide chain in the case of scFv sequences.

The CDRs contribute to the formation of the antigen-binding, or more specifically, epitope binding site of antibodies. “Epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope.

The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and nonconformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning.” As outlined below, the invention not only includes the enumerated antigen binding domains and antibodies herein, but those that compete for binding with the epitopes bound by the enumerated antigen binding domains.

Thus, the present invention provides different antibody domains. As described herein and known in the art, the heterodimeric antibodies of the invention comprise different domains within the heavy and light chains, which can be overlapping as well. These domains include, but are not limited to, the Fc domain, the CH1 domain, the CH2 domain, the CH3 domain, the hinge domain, the heavy constant domain (CH1-hinge-Fc domain or CH1-hinge-CH2-CH3), the variable heavy domain, the variable light domain, the light constant domain, Fab domains and scFv domains.

Thus, the “Fc domain” includes the -CH2-CH3 domain, and optionally a hinge domain (-hinge domain-CH2-CH3) (again, with many embodiments relying on a hinge domain from human IgG1 with a C220S variant). In the embodiments herein, particularly for the “1+1” format, when a scFv is attached to an Fc domain, it is the C-terminus of the scFv construct that is attached to all or part of the hinge of the Fc domain; for example, it is generally attached to the sequence EPKS (SEQ ID NO: 473) which is the beginning of the hinge. In some cases, for example in the “2+1” format, the domain linker can be a combination of flexible linker amino acids as well as part or all of the hinge; for example, SEQ ID NO:29 is such an example.

The embodiments of the invention comprise at least one scFv domain, which, while not naturally occurring, generally includes a variable heavy domain and a variable light domain, linked together by a scFv linker. As outlined herein, while the scFv domain is generally from N- to C-terminus oriented as VH-scFv linker-VL, this can be reversed for any of the scFv domains (or those constructed using VH and VL sequences from Fabs), to VL-scFv linker-VH, with optional linkers at one or both ends depending on the format (see generally FIGS. 13 and 42).

### (a) Linkers

As shown herein, there are a number of suitable linkers (for use as either domain linkers or scFv linkers) that can be used to covalently attach the recited domains, including traditional peptide bonds, generated by recombinant techniques. In some embodiments, the linker peptide may predominantly include the following amino acid residues: Gly, Ser, Ala, or Thr. The linker peptide should have a length that is adequate to link two molecules in such a way that they assume the correct conformation relative to one another so that they retain the desired activity. In one embodiment, the linker is from about 1 to 50 amino acids in length, preferably about 1 to 30 amino acids in length. In one embodiment, linkers of 1 to 20 amino acids in length may be used, with from about 5 to about 10 amino acids finding use in some embodiments. Useful linkers include glycine-serine polymers, including for example (GS)n, (GSGGS)n (SEQ ID NO: 474), (GGGGS)n (SEQ ID NO: 475), and (GGGS)n (SEQ ID NO: 476), where n is an integer of at least one (and generally from 3 to 4), glycine-alanine polymers, alanine-serine polymers, and other flexible linkers, some of which are shown in FIG. 5. Alternatively, a variety of nonproteinaceous polymers, including but not limited to polyethylene glycol (PEG), polypropylene glycol, polyoxyalkylenes, or copolymers of polyethylene glycol and polypropylene glycol, may find use as linkers.

Other linker sequences may include any sequence of any length of CL/CH1 domain but not all residues of CL/CH1 domain; for example the first 5-12 amino acid residues of the CL/CH1 domains. Linkers can be derived from immunoglobulin light chain, for example Cκ or Cλ. Linkers can be derived from immunoglobulin heavy chains of any isotype, including for example Cγ1, Cγ2, Cγ3, Cγ4, Cα1, Cα2, Cδ, Cϵ, and Cμ. Linker sequences may also be derived from other proteins such as Ig-like proteins (e.g. TCR, FcR, KIR), hinge region-derived sequences, and other natural sequences from other proteins.

In some embodiments, the linker is a “domain linker”, used to link any two domains as outlined herein together. For example, in FIG. 42F, there may be a domain linker that attaches the C-terminus of the CH1 domain of the Fab to the N-terminus of the scFv, with another optional domain linker attaching the C-terminus of the scFv to the CH2 domain (although in many embodiments the hinge is used as this domain linker). While any suitable linker can be used, many embodiments utilize a glycine-serine polymer as the domain linker, including for example (GS)n, (GSGGS)n (SEQ ID NO: 474), (GGGGS)n (SEQ ID NO: 475), and (GGGS)n (SEQ ID NO: 476), where n is an integer of at least one (and generally from 3 to 4 to 5) as well as any peptide sequence that allows for recombinant attachment of the two domains with sufficient length and flexibility to allow each domain to retain its biological function. In some cases, and with attention being paid to “strandedness”, as outlined below, charged domain linkers, as used in some embodiments of scFv linkers can be used.

With particular reference to the domain linker used to attach the scFv domain to the Fc domain in the “2+1” format, there are several domain linkers that find particular use, including SEQ ID NO:28, SEQ ID NO:29, SEQ ID NO:30, SEQ ID NO:31.

In some embodiments, the linker is a “scFv linker”, used to covalently attach the VH and VL domains as discussed herein. In many cases, the scFv linker is a charged scFv linker, a number of which are shown in FIG. 5. Accordingly, the present invention further provides charged scFv linkers, to facilitate the separation in pI between a first and a second monomer. That is, by incorporating a charged scFv linker, either positive or negative (or both, in the case of scaffolds that use scFvs on different monomers), this allows the monomer comprising the charged linker to alter the pI without making further changes in the Fc domains. These charged linkers can be substituted into any scFv containing standard linkers. Again, as will be appreciated by those in the art, charged scFv linkers are used on the correct “strand” or monomer, according to the desired changes in pI. For example, as discussed herein, to make 1+1 Fab-scFv-Fc format heterodimeric antibody, the original pI of the Fv region for each of the desired antigen binding domains are calculated, and one is chosen to make an scFv, and depending on the pI, either positive or negative linkers are chosen.

Charged domain linkers can also be used to increase the pI separation of the monomers of the invention as well, and thus those included in FIG. 5 can be used in any embodiment herein where a linker is utilized.

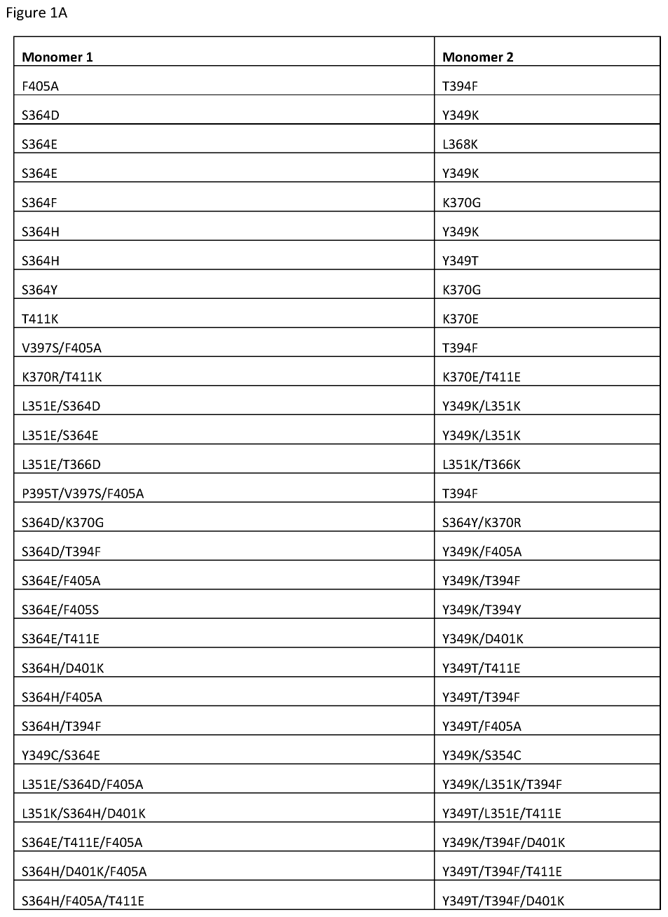

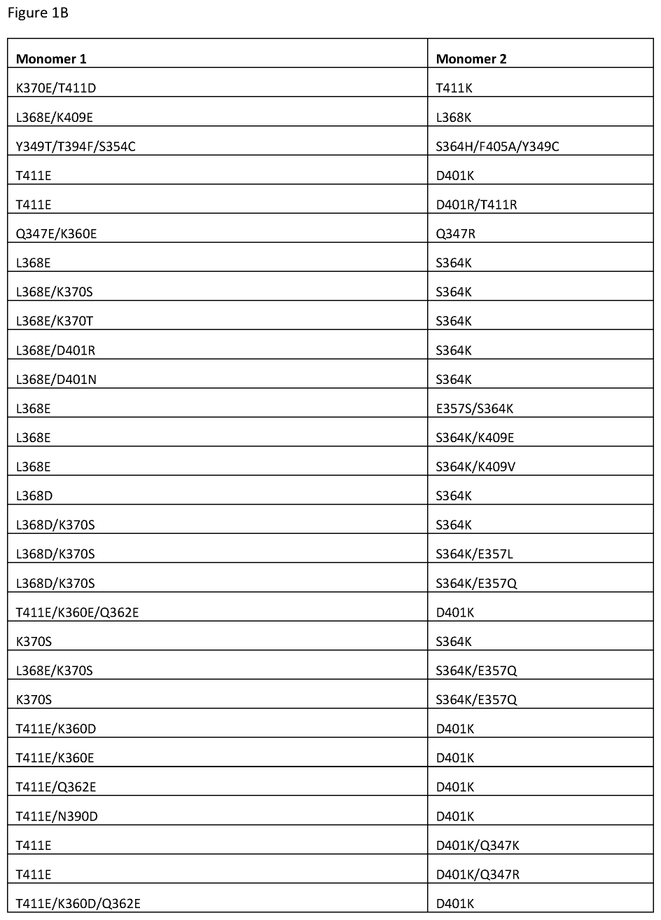

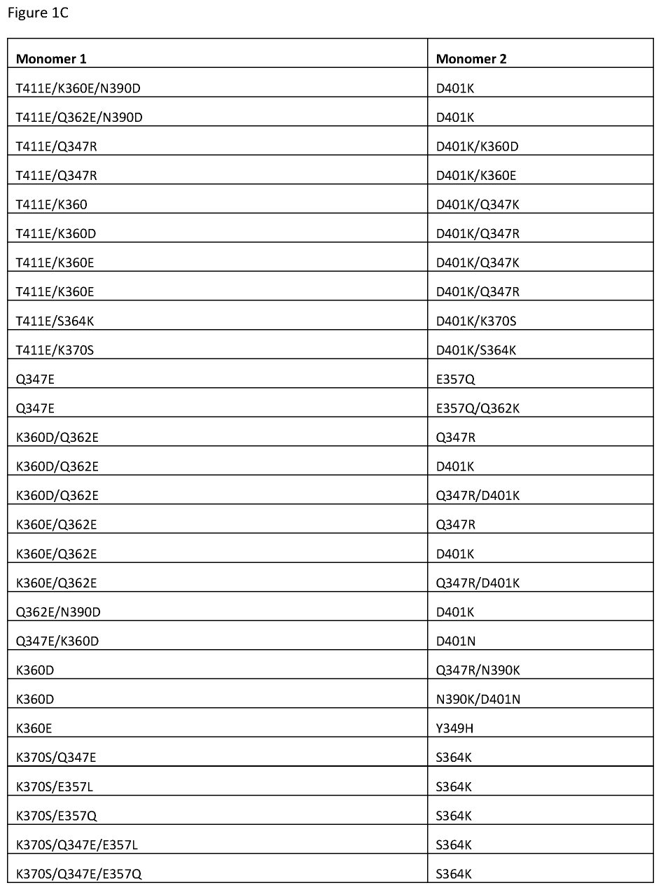

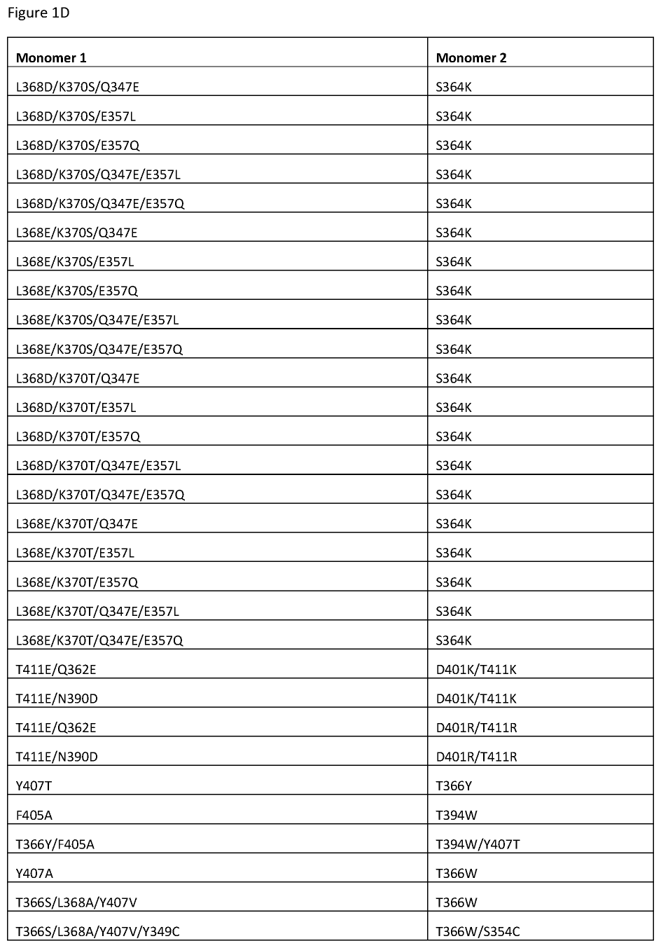

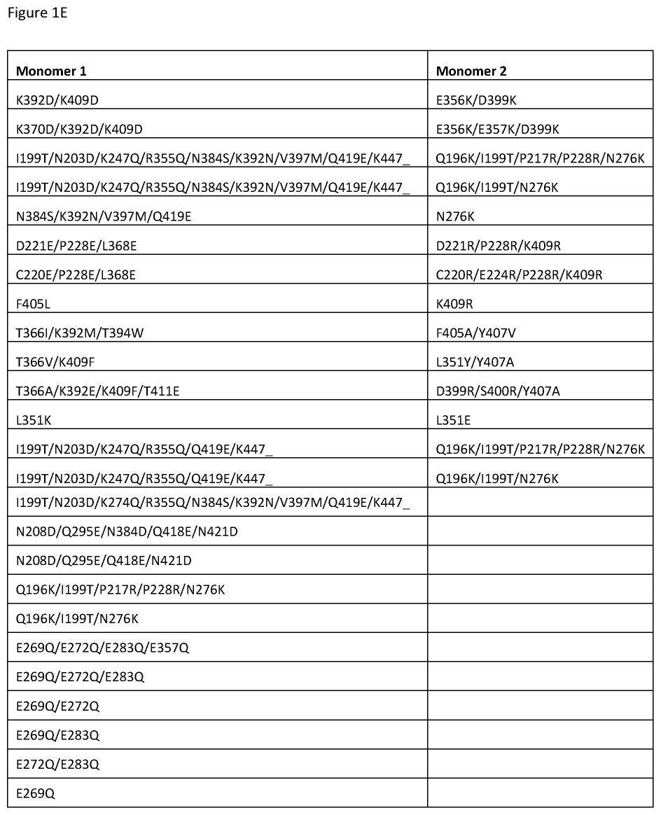

In particular, the formats depicted in FIG. 1 are antibodies, usually referred to as “heterodimeric antibodies”, meaning that the protein has at least two associated Fc sequences self-assembled into a heterodimeric Fc domain and at least two Fv regions, whether as Fabs or as scFvs.

### F. Chimeric and Humanized Antibodies

In certain embodiments, the antibodies of the invention comprise a heavy chain variable region from a particular germline heavy chain immunoglobulin gene and/or a light chain variable region from a particular germline light chain immunoglobulin gene. For example, such antibodies may comprise or consist of a human antibody comprising heavy or light chain variable regions that are “the product of” or “derived from” a particular germline sequence. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody (using the methods outlined herein). A human antibody that is “the product of” or “derived from” a particular human germline immunoglobulin sequence may contain amino acid differences as compared to the germline sequence, due to, for example, naturally-occurring somatic mutations or intentional introduction of site-directed mutation. However, a humanized antibody typically is at least 90% identical in amino acids sequence to an amino acid sequence encoded by a human germline immunoglobulin gene and contains amino acid residues that identify the antibody as being derived from human sequences when compared to the germline immunoglobulin amino acid sequences of other species (e.g., murine germline sequences). In certain cases, a humanized antibody may be at least 95, 96, 97, 98 or 99%, or even at least 96%, 97%, 98%, or 99% identical in amino acid sequence to the amino acid sequence encoded by the germline immunoglobulin gene. Typically, a humanized antibody derived from a particular human germline sequence will display no more than 10-20 amino acid differences from the amino acid sequence encoded by the human germline immunoglobulin gene (prior to the introduction of any skew, pI and ablation variants herein; that is, the number of variants is generally low, prior to the introduction of the variants of the invention). In certain cases, the humanized antibody may display no more than 5, or even no more than 4, 3, 2, or 1 amino acid difference from the amino acid sequence encoded by the germline immunoglobulin gene (again, prior to the introduction of any skew, pI and ablation variants herein; that is, the number of variants is generally low, prior to the introduction of the variants of the invention).

In one embodiment, the parent antibody has been affinity matured, as is known in the art. Structure-based methods may be employed for humanization and affinity maturation, for example as described in U.S. Ser. No. 11/004,590. Selection based methods may be employed to humanize and/or affinity mature antibody variable regions, including but not limited to methods described in Wu et al., 1999, J. Mol. Biol. 294:151-162; Baca et al., 1997, J. Biol. Chem. 272(16):10678-10684; Rosok et al., 1996, J. Biol. Chem. 271(37): 22611-22618; Rader et al., 1998, Proc. Natl. Acad. Sci. USA 95: 8910-8915; Krauss et al., 2003, Protein Engineering 16(10):753-759, all entirely incorporated by reference. Other humanization methods may involve the grafting of only parts of the CDRs, including but not limited to methods described in U.S. Ser. No. 09/810,510; Tan et al., 2002, J. Immunol. 169:1119-1125; De Pascalis et al., 2002, J. Immunol. 169:3076-3084, all entirely incorporated by reference.

### G. Heterodimeric Antibodies

Accordingly, in some embodiments, the subject antibody is a heterodimeric antibody that relies on the use of two different heavy chain variant Fc sequences. Such an antibody will self-assemble to form a heterodimeric Fc domain and heterodimeric antibody.

The present invention is directed to novel constructs to provide heterodimeric antibodies that allow binding to more than one antigen or ligand, e.g. to allow for bispecific binding (e.g., anti-CLDN18.2 and anti-CD3 binding). The heterodimeric antibody constructs are based on the self-assembling nature of the two Fc domains of the heavy chains of antibodies, e.g. two “monomers” that assemble into a “dimer”. Heterodimeric antibodies are made by altering the amino acid sequence of each monomer as more fully discussed below. Thus, the present invention is generally directed to the creation of heterodimeric antibodies which can co-engage antigens (e.g., CLDN18.2 and CD3) in several ways, relying on amino acid variants in the constant regions that are different on each chain to promote heterodimeric formation and/or allow for ease of purification of heterodimers over the homodimers.

Thus, the present invention provides bispecific antibodies. In some embodiments, the present invention provides bispecific antibodies that include an CLDN18.2 binding domain. In some embodiments, the bispecific antibody is an anti-CLDN18.2 x anti-CD3 bispecific antibody. An ongoing problem in antibody technologies is the desire for “bispecific” antibodies that bind to two different antigens simultaneously, in general thus allowing the different antigens to be brought into proximity and resulting in new functionalities and new therapies. In general, these antibodies are made by including genes for each heavy and light chain into the host cells. This generally results in the formation of the desired heterodimer (A-B), as well as the two homodimers (A-A and B-B (not including the light chain heterodimeric issues)). However, a major obstacle in the formation of bispecific antibodies is the difficulty in purifying the heterodimeric antibodies away from the homodimeric antibodies and/or biasing the formation of the heterodimer over the formation of the homodimers.

There are a number of mechanisms that can be used to generate the heterodimers of the present invention. In addition, as will be appreciated by those in the art, these mechanisms can be combined to ensure high heterodimerization. Thus, amino acid variants that lead to the production of heterodimers are referred to as “heterodimerization variants”. As discussed below, heterodimerization variants can include steric variants (e.g. the “knobs and holes” or “skew” variants described below and the “charge pairs” variants described below) as well as “pI variants”, which allows purification of homodimers away from heterodimers. As is generally described in WO2014/145806, hereby incorporated by reference in its entirety and specifically as below for the discussion of “heterodimerization variants”, useful mechanisms for heterodimerization include “knobs and holes” (“KIH”; sometimes herein as “skew” variants (see discussion in WO2014/145806), “electrostatic steering” or “charge pairs” as described in WO2014/145806, pI variants as described in WO2014/145806, and general additional Fc variants as outlined in WO2014/145806 and below.

In the present invention, there are several basic mechanisms that can lead to ease of purifying heterodimeric antibodies; one relies on the use of pI variants, such that each monomer has a different pI, thus allowing the isoelectric purification of A-A, A-B and B-B dimeric proteins. Alternatively, some scaffold formats, such as the “1+1 Fab-scFv-Fc” format, also allows separation on the basis of size. As is further outlined below, it is also possible to “skew” the formation of heterodimers over homodimers. Thus, a combination of steric heterodimerization variants and pI or charge pair variants find particular use in the invention.

In general, embodiments of particular use in the present invention rely on sets of variants that include skew variants, which encourage heterodimerization formation over homodimerization formation, coupled with pI variants, which increase the pI difference between the two monomers to facilitate purification of heterodimers away from homodimers.

Additionally, as more fully outlined below, depending on the format of the heterodimer antibody, pI variants can be either contained within the constant and/or Fc domains of a monomer, or charged linkers, either domain linkers or scFv linkers, can be used. That is, scaffolds that utilize scFv(s) such as the “1+1 Fab-scFv-Fc” format can include charged scFv linkers (either positive or negative), that give a further pI boost for purification purposes. As will be appreciated by those in the art, some 1+1 Fab-scFv-Fc formats are useful with just charged scFv linkers and no additional pI adjustments, although the invention does provide pI variants that are on one or both of the monomers, and/or charged domain linkers as well. In addition, additional amino acid engineering for alternative functionalities may also confer pI changes, such as Fc, FcRn and KO variants.

In the present invention that utilizes pI as a separation mechanism to allow the purification of heterodimeric proteins, amino acid variants can be introduced into one or both of the monomer polypeptides; that is, the pI of one of the monomers (referred to herein for simplicity as “monomer A”) can be engineered away from monomer B, or both monomer A and B change be changed, with the pI of monomer A increasing and the pI of monomer B decreasing. As is outlined more fully below, the pI changes of either or both monomers can be done by removing or adding a charged residue (e.g. a neutral amino acid is replaced by a positively or negatively charged amino acid residue, e.g. glycine to glutamic acid), changing a charged residue from positive or negative to the opposite charge (aspartic acid to lysine) or changing a charged residue to a neutral residue (e.g. loss of a charge; lysine to serine.). A number of these variants are shown in the Figures.

Accordingly, this embodiment of the present invention provides for creating a sufficient change in pI in at least one of the monomers such that heterodimers can be separated from homodimers. As will be appreciated by those in the art, and as discussed further below, this can be done by using a “wild type” heavy chain constant region and a variant region that has been engineered to either increase or decrease it's pI (wt A−+B or wt A −−B), or by increasing one region and decreasing the other region (A+−B− or A−B+).

Thus, in general, a component of some embodiments of the present invention are amino acid variants in the constant regions of antibodies that are directed to altering the isoelectric point (pI) of at least one, if not both, of the monomers of a dimeric protein to form “pI antibodies”) by incorporating amino acid substitutions (“pI variants” or “pI substitutions”) into one or both of the monomers. As shown herein, the separation of the heterodimers from the two homodimers can be accomplished if the pIs of the two monomers differ by as little as 0.1 pH unit, with 0.2, 0.3, 0.4 and 0.5 or greater all finding use in the present invention.

As will be appreciated by those in the art, the number of pI variants to be included on each or both monomer(s) to get good separation will depend in part on the starting pI of the components, for example in the 1+1 Fab-scFv-Fc format, the starting pI of the scFv and Fab of interest. That is, to determine which monomer to engineer or in which “direction” (e.g. more positive or more negative), the Fv sequences of the two target antigens are calculated and a decision is made from there. As is known in the art, different Fvs will have different starting pIs which are exploited in the present invention. In general, as outlined herein, the pIs are engineered to result in a total pI difference of each monomer of at least about 0.1 logs, with 0.2 to 0.5 being preferred as outlined herein.

Furthermore, as will be appreciated by those in the art and outlined herein, in some embodiments, heterodimers can be separated from homodimers on the basis of size. As shown in FIG. 1, for example, several of the formats allow separation of heterodimers and homodimers on the basis of size.

In the case where pI variants are used to achieve heterodimerization, by using the constant region(s) of the heavy chain(s), a more modular approach to designing and purifying bispecific proteins, including antibodies, is provided. Thus, in some embodiments, heterodimerization variants (including skew and purification heterodimerization variants) are not included in the variable regions, such that each individual antibody must be engineered. In addition, in some embodiments, the possibility of immunogenicity resulting from the pI variants is significantly reduced by importing pI variants from different IgG isotypes such that pI is changed without introducing significant immunogenicity. Thus, an additional problem to be solved is the elucidation of low pI constant domains with high human sequence content, e.g. the minimization or avoidance of non-human residues at any particular position.

A side benefit that can occur with this pI engineering is also the extension of serum half-life and increased FcRn binding. That is, as described in U.S. Ser. No. 13/194,904 (incorporated by reference in its entirety), lowering the pI of antibody constant domains (including those found in antibodies and Fc fusions) can lead to longer serum retention in vivo. These pI variants for increased serum half-life also facilitate pI changes for purification.

In addition, it should be noted that the pI variants of the heterodimerization variants give an additional benefit for the analytics and quality control process of bispecific antibodies, as the ability to either eliminate, minimize and distinguish when homodimers are present is significant. Similarly, the ability to reliably test the reproducibility of the heterodimeric antibody production is important.

### Heterodimerization Variants

The present invention provides heterodimeric proteins, including heterodimeric antibodies in a variety of formats, which utilize heterodimeric variants to allow for heterodimeric formation and/or purification away from homodimers.

There are a number of suitable pairs of sets of heterodimerization skew variants. These variants come in “pairs” of “sets”. That is, one set of the pair is incorporated into the first monomer and the other set of the pair is incorporated into the second monomer. It should be noted that these sets do not necessarily behave as “knobs in holes” variants, with a one-to-one correspondence between a residue on one monomer and a residue on the other; that is, these pairs of sets form an interface between the two monomers that encourages heterodimer formation and discourages homodimer formation, allowing the percentage of heterodimers that spontaneously form under biological conditions to be over 90%, rather than the expected 50% (25% homodimer A/A:50% heterodimer A/B:25% homodimer B/B).

### Steric Variants

In some embodiments, the formation of heterodimers can be facilitated by the addition of steric variants. That is, by changing amino acids in each heavy chain, different heavy chains are more likely to associate to form the heterodimeric structure than to form homodimers with the same Fc amino acid sequences. Suitable steric variants are included in FIG. 1.

One mechanism is generally referred to in the art as “knobs and holes”, referring to amino acid engineering that creates steric influences to favor heterodimeric formation and disfavor homodimeric formation can also optionally be used; this is sometimes referred to as “knobs and holes”, as described in U.S. Ser. No. 61/596,846, Ridgway et al., Protein Engineering 9(7):617 (1996); Atwell et al., J. Mol. Biol. 1997 270:26; U.S. Pat. No. 8,216,805, all of which are hereby incorporated by reference in their entirety. The Figures identify a number of “monomer A-monomer B” pairs that rely on “knobs and holes”. In addition, as described in Merchant et al., Nature Biotech. 16:677 (1998), these “knobs and hole” mutations can be combined with disulfide bonds to skew formation to heterodimerization.

An additional mechanism that finds use in the generation of heterodimers is sometimes referred to as “electrostatic steering” as described in Gunasekaran et al., J. Biol. Chem. 285(25):19637 (2010), hereby incorporated by reference in its entirety. This is sometimes referred to herein as “charge pairs”. In this embodiment, electrostatics are used to skew the formation towards heterodimerization. As those in the art will appreciate, these may also have an effect on pI, and thus on purification, and thus could in some cases also be considered pI variants. However, as these were generated to force heterodimerization and were not used as purification tools, they are classified as “steric variants”. These include, but are not limited to, D221E/P228E/L368E paired with D221R/P228R/K409R (e.g. these are “monomer corresponding sets) and C220E/P228E/368E paired with C220R/E224R/P228R/K409R.

Additional monomer A and monomer B variants that can be combined with other variants, optionally and independently in any amount, such as pI variants outlined herein or other steric variants that are shown in FIG. 37 of US 2012/0149876, the figure and legend and SEQ ID NOs of which are incorporated expressly by reference herein.

In some embodiments, the steric variants outlined herein can be optionally and independently incorporated with any pI variant (or other variants such as Fc variants, FcRn variants, etc.) into one or both monomers, and can be independently and optionally included or excluded from the proteins of the invention.

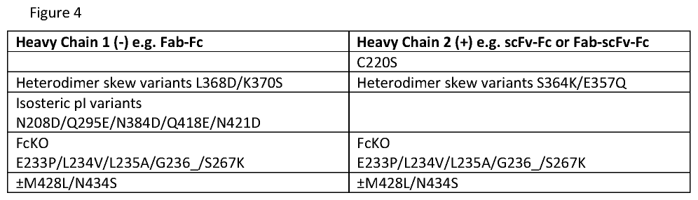

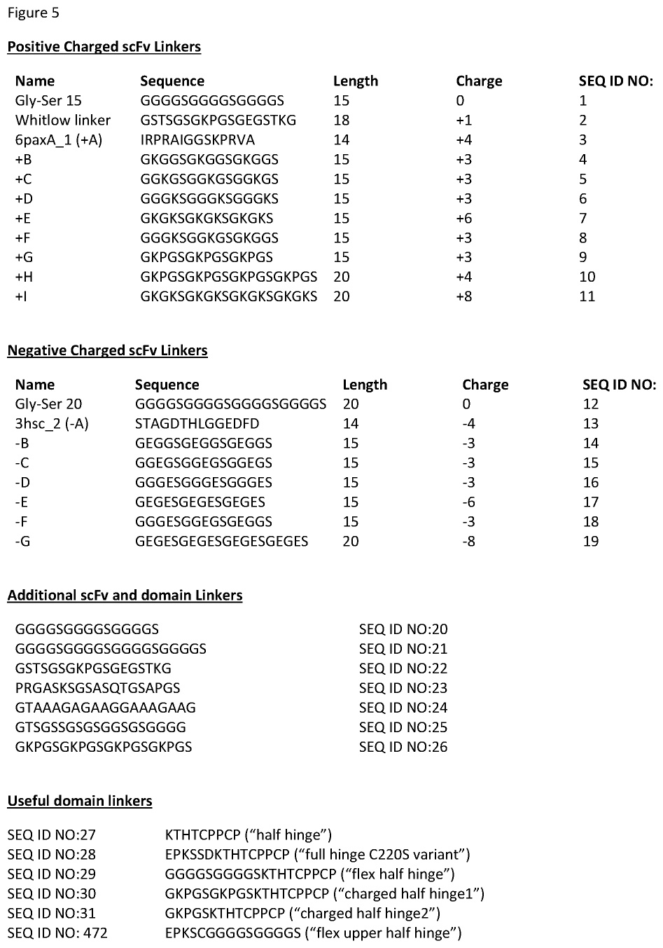

A list of suitable skew variants is found in FIG. 1, with FIG. 4 showing some pairs of particular utility in many embodiments. Of particular use in many embodiments are the pairs of sets including, but not limited to, S364K/E357Q:L368D/K370S; L368D/K370S:S364K; L368E/K370S:S364K; T411T/E360E/Q362E:D401K; L368D/K370S:S364K/E357L and K370S:S364K/E357Q. In terms of nomenclature, the pair “S364K/E357Q:L368D/K370S” means that one of the monomers has the double variant set S364K/E357Q and the other has the double variant set L368D/K370S.

### pI (Isoelectric Point) Variants for Heterodimers

In general, as will be appreciated by those in the art, there are two general categories of pI variants: those that increase the pI of the protein (basic changes) and those that decrease the pI of the protein (acidic changes). As described herein, all combinations of these variants can be done: one monomer may be wild type, or a variant that does not display a significantly different pI from wild-type, and the other can be either more basic or more acidic. Alternatively, each monomer is changed, one to more basic and one to more acidic.

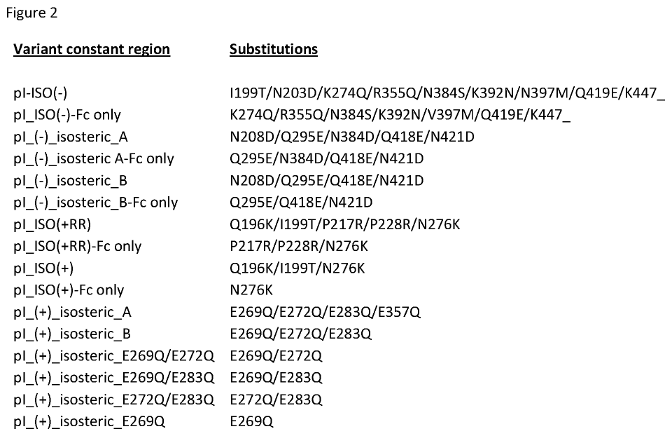

Preferred combinations of pI variants are shown in FIGS. 1 and 2. As outlined herein and shown in the figures, these changes are shown relative to IgG1, but all isotypes can be altered this way, as well as isotype hybrids. In the case where the heavy chain constant domain is from IgG2-4, R133E and R133Q can also be used.

In one embodiment, for example in the FIG. 42A, E, F, G, H and I formats, a preferred combination of pI variants has one monomer (the negative Fab side) comprising 208D/295E/384D/418E/421D variants (N208D/Q295E/N384D/Q418E/N421D when relative to human IgG1) and a second monomer (the positive scFv side) comprising a positively charged scFv linker, including (GKPGS)₄ (SEQ ID NO: 10). However, as will be appreciated by those in the art, the first monomer includes a CH1 domain, including position 208. Accordingly, in constructs that do not include a CH1 domain (for example for antibodies that do not utilize a CH1 domain on one of the domains, for example in a dual scFv format or a “one armed” format such as those depicted in FIG. 42B, C or D), a preferred negative pI variant Fc set includes 295E/384D/418E/421D variants (Q295E/N384D/Q418E/N421D when relative to human IgG1).

Accordingly, in some embodiments, one monomer has a set of substitutions from FIG. 2 and the other monomer has a charged linker (either in the form of a charged scFv linker because that monomer comprises an scFv or a charged domain linker, as the format dictates, which can be selected from those depicted in FIG. 5).

### Isotypic Variants

In addition, many embodiments of the invention rely on the “importation” of pI amino acids at particular positions from one IgG isotype into another, thus reducing or eliminating the possibility of unwanted immunogenicity being introduced into the variants. A number of these are shown in FIG. 21 of US Publ. 2014/0370013, hereby incorporated by reference. That is, IgG1 is a common isotype for therapeutic antibodies for a variety of reasons, including high effector function. However, the heavy constant region of IgG1 has a higher pI than that of IgG2 (8.10 versus 7.31). By introducing IgG2 residues at particular positions into the IgG1 backbone, the pI of the resulting monomer is lowered (or increased) and additionally exhibits longer serum half-life. For example, IgG1 has a glycine (pI 5.97) at position 137, and IgG2 has a glutamic acid (pI 3.22); importing the glutamic acid will affect the pI of the resulting protein. As is described below, a number of amino acid substitutions are generally required to significant affect the pI of the variant antibody. However, it should be noted as discussed below that even changes in IgG2 molecules allow for increased serum half-life.

In other embodiments, non-isotypic amino acid changes are made, either to reduce the overall charge state of the resulting protein (e.g. by changing a higher pI amino acid to a lower pI amino acid), or to allow accommodations in structure for stability, etc. as is more further described below.

In addition, by pI engineering both the heavy and light constant domains, significant changes in each monomer of the heterodimer can be seen. As discussed herein, having the pIs of the two monomers differ by at least 0.5 can allow separation by ion exchange chromatography or isoelectric focusing, or other methods sensitive to isoelectric point.

### Calculating pI

The pI of each monomer can depend on the pI of the variant heavy chain constant domain and the pI of the total monomer, including the variant heavy chain constant domain and the fusion partner. Thus, in some embodiments, the change in pI is calculated on the basis of the variant heavy chain constant domain, using the chart in the FIG. 19 of US Pub. 2014/0370013. As discussed herein, which monomer to engineer is generally decided by the inherent pI of the Fv and scaffold regions. Alternatively, the pI of each monomer can be compared.

### pI Variants That Also Confer Better FcRn In Vivo Binding

In the case where the pI variant decreases the pI of the monomer, they can have the added benefit of improving serum retention in vivo.

Although still under examination, Fc regions are believed to have longer half-lives in vivo, because binding to FcRn at pH 6 in an endosome sequesters the Fc (Ghetie and Ward, 1997 Immunol Today. 18(12): 592-598, entirely incorporated by reference). The endosomal compartment then recycles the Fc to the cell surface. Once the compartment opens to the extracellular space, the higher pH, ˜7.4, induces the release of Fc back into the blood. In mice, Dall' Acqua et al. showed that Fc mutants with increased FcRn binding at pH 6 and pH 7.4 actually had reduced serum concentrations and the same half-life as wild-type Fc (Dall' Acqua et al. 2002, J. Immunol. 169:5171-5180, entirely incorporated by reference). The increased affinity of Fc for FcRn at pH 7.4 is thought to forbid the release of the Fc back into the blood. Therefore, the Fc mutations that will increase Fc's half-life in vivo will ideally increase FcRn binding at the lower pH while still allowing release of Fc at higher pH. The amino acid histidine changes its charge state in the pH range of 6.0 to 7.4. Therefore, it is not surprising to find His residues at important positions in the Fc/FcRn complex.

Recently it has been suggested that antibodies with variable regions that have lower isoelectric points may also have longer serum half-lives (Igawa et al., 2010 PEDS. 23(5): 385-392, entirely incorporated by reference). However, the mechanism of this is still poorly understood. Moreover, variable regions differ from antibody to antibody. Constant region variants with reduced pI and extended half-life would provide a more modular approach to improving the pharmacokinetic properties of antibodies, as described herein.

### Additional Fc Variants for Additional Functionality

In addition to pI amino acid variants, there are a number of useful Fc amino acid modification that can be made for a variety of reasons, including, but not limited to, altering binding to one or more FcγR receptors, altered binding to FcRn receptors, etc.

Accordingly, the proteins of the invention can include amino acid modifications, including the heterodimerization variants outlined herein, which includes the pI variants and steric variants. Each set of variants can be independently and optionally included or excluded from any particular heterodimeric protein.

FcγR Variants

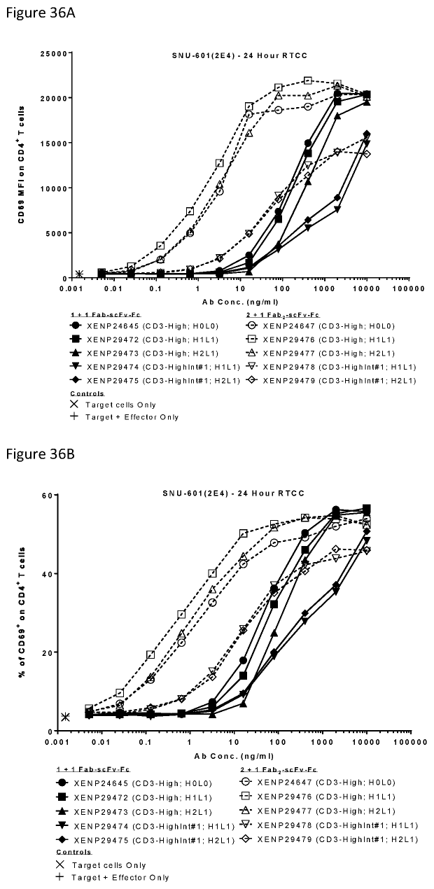

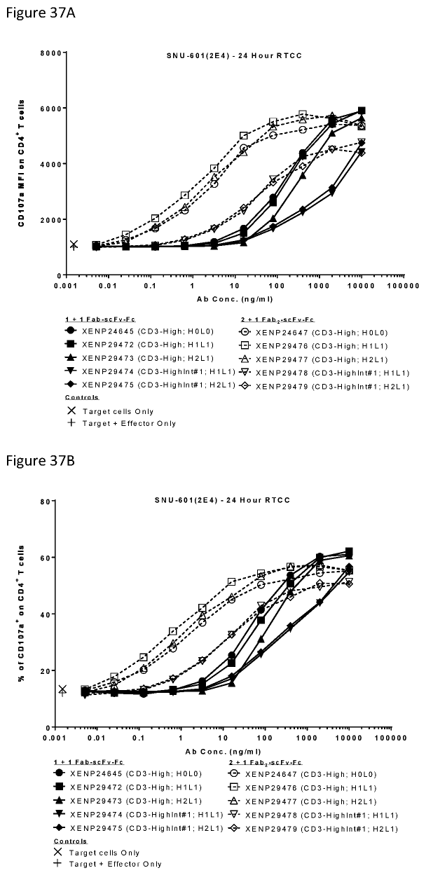

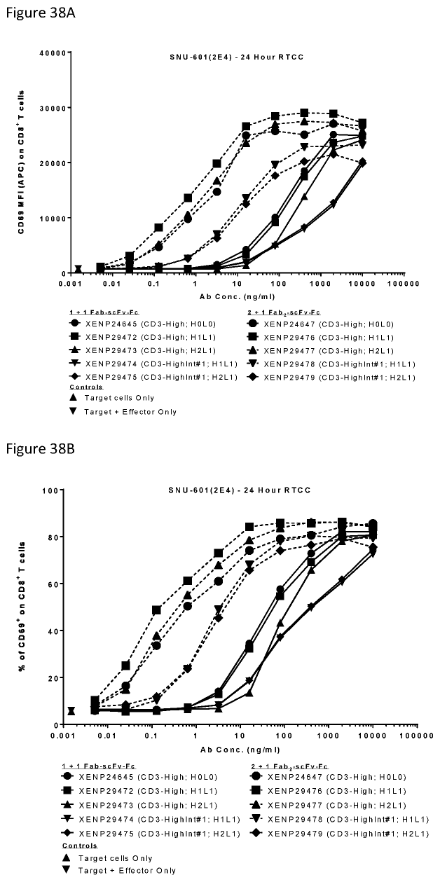

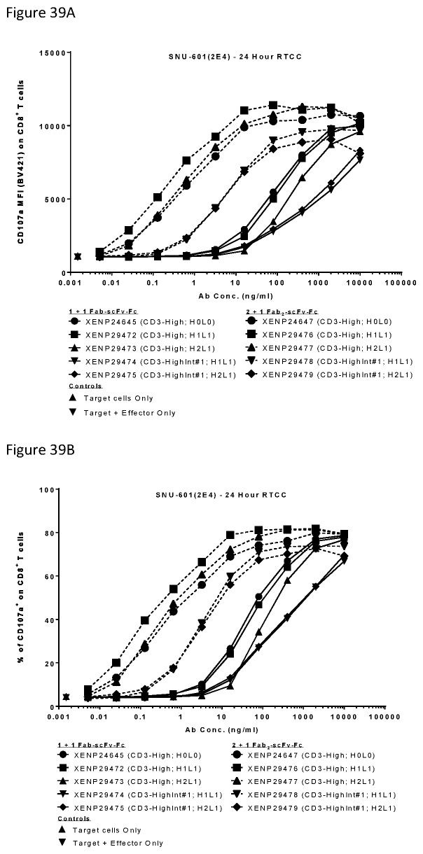

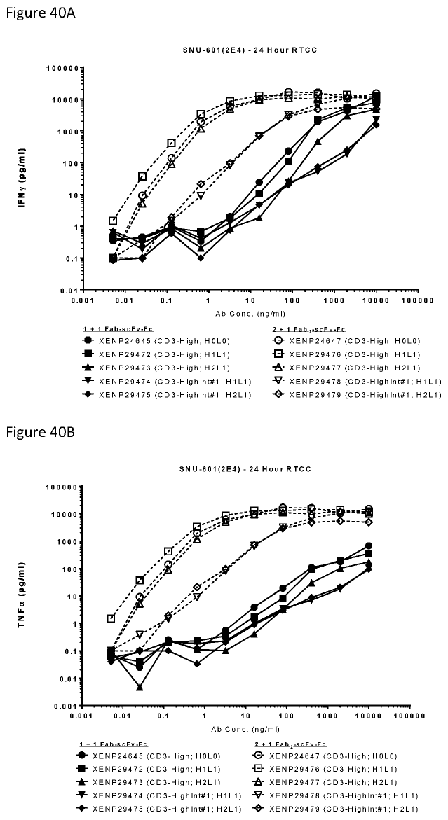

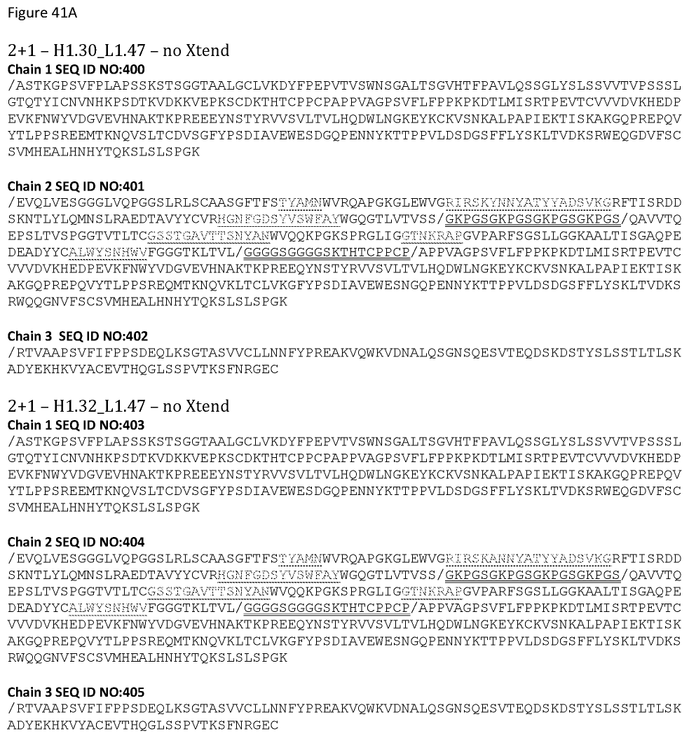

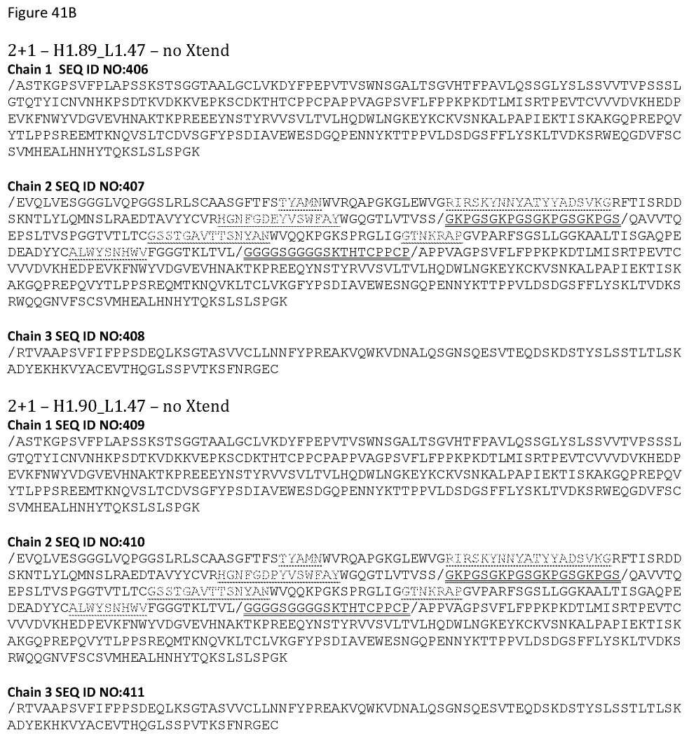

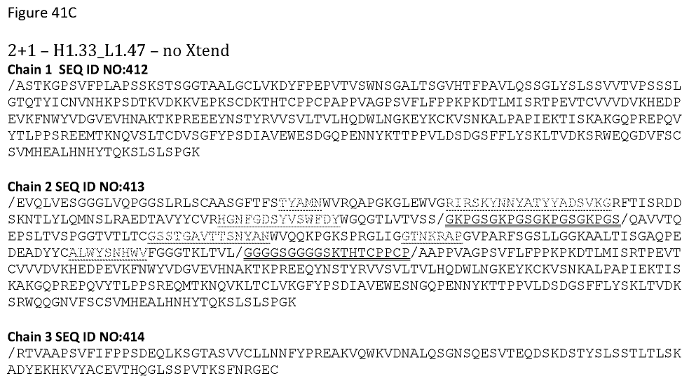

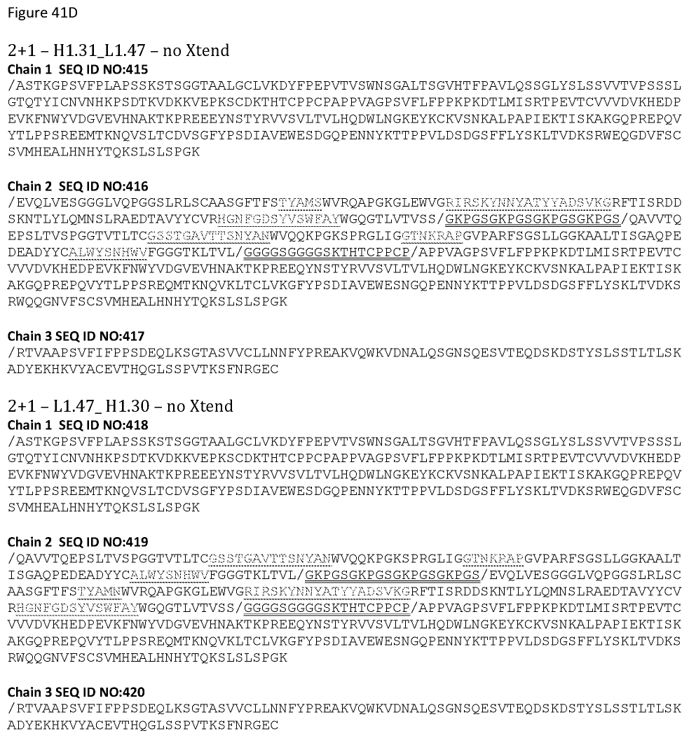

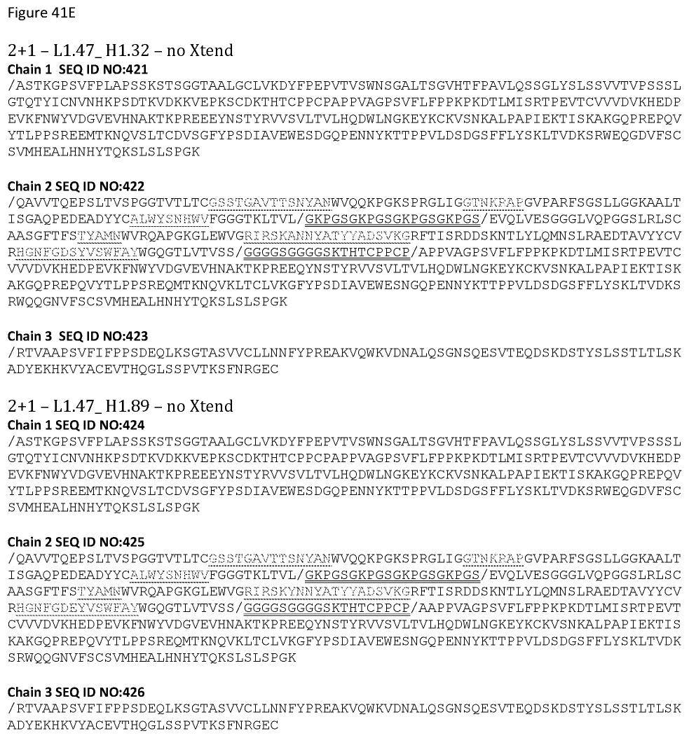

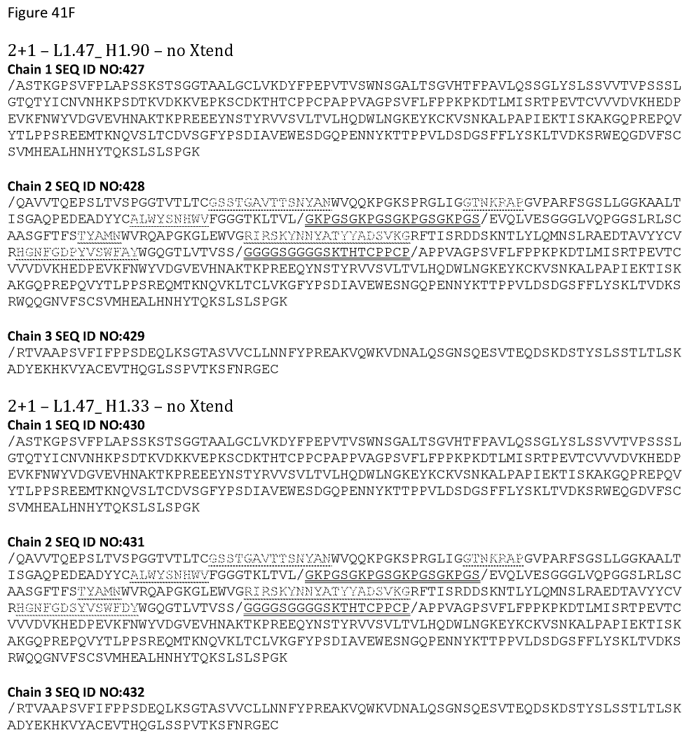

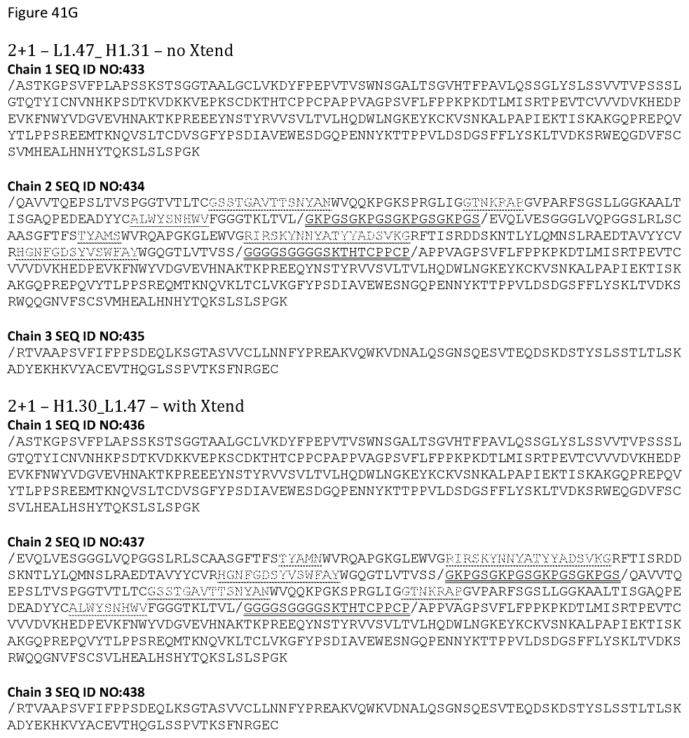

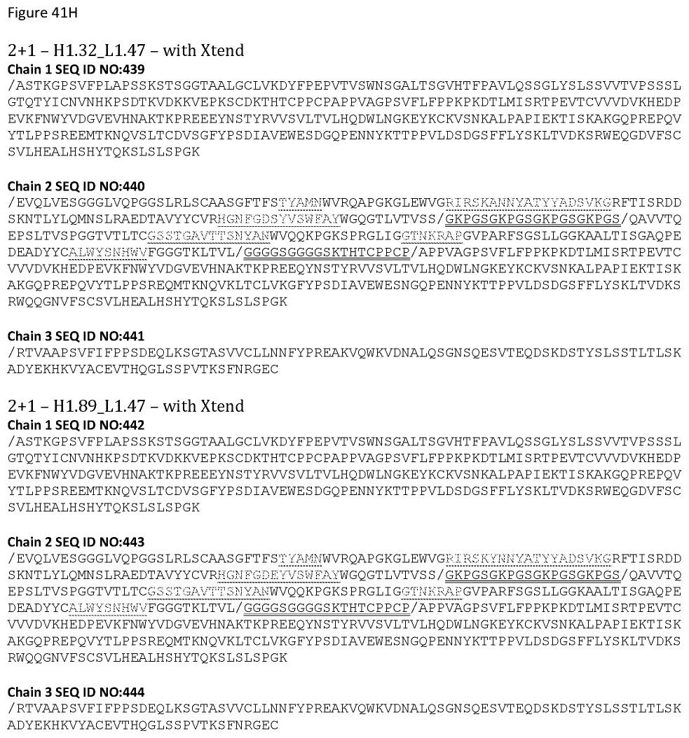

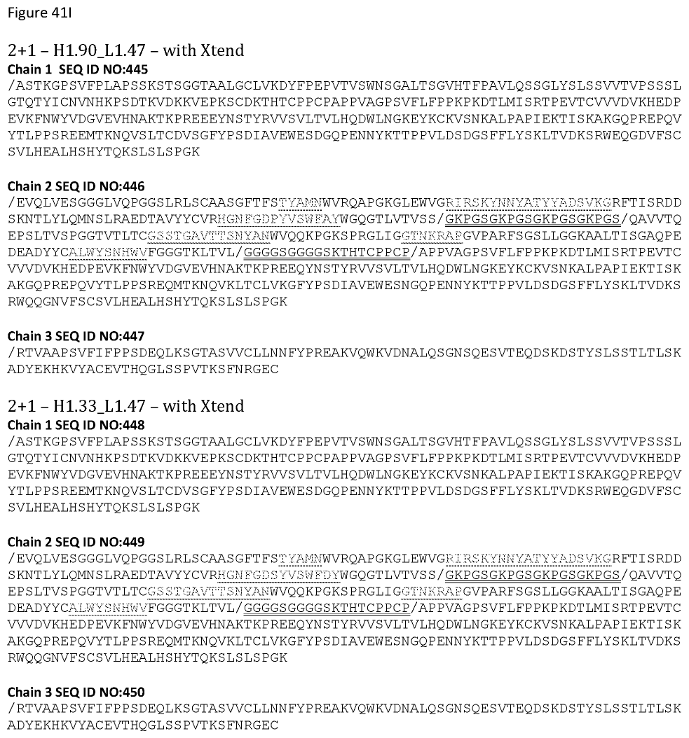

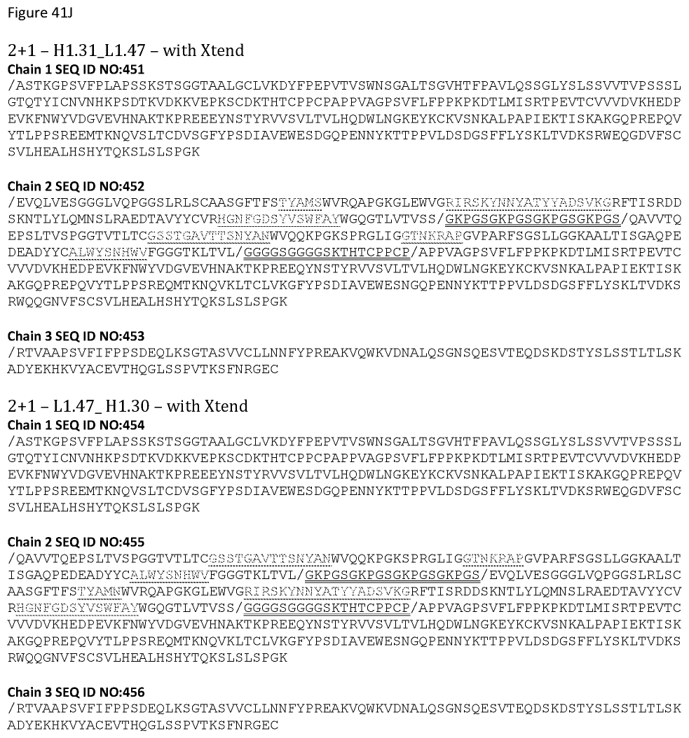

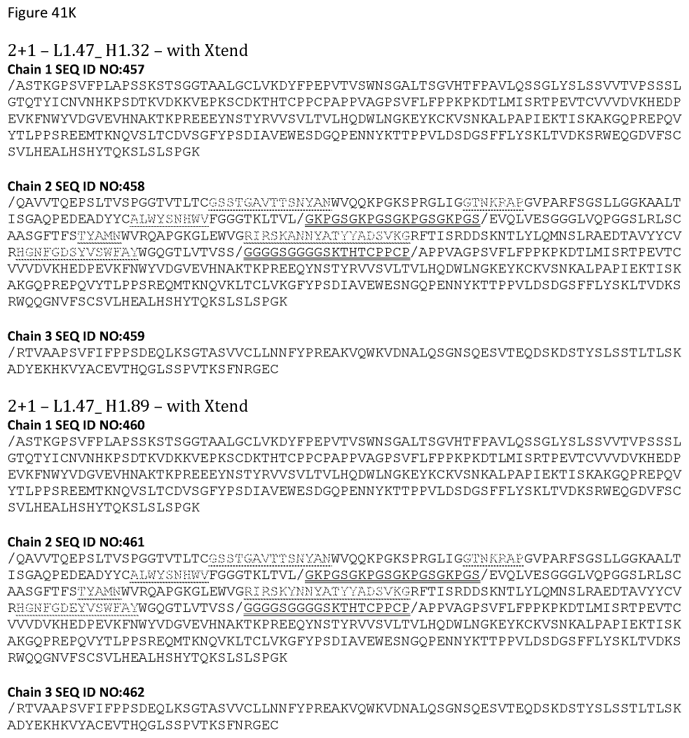

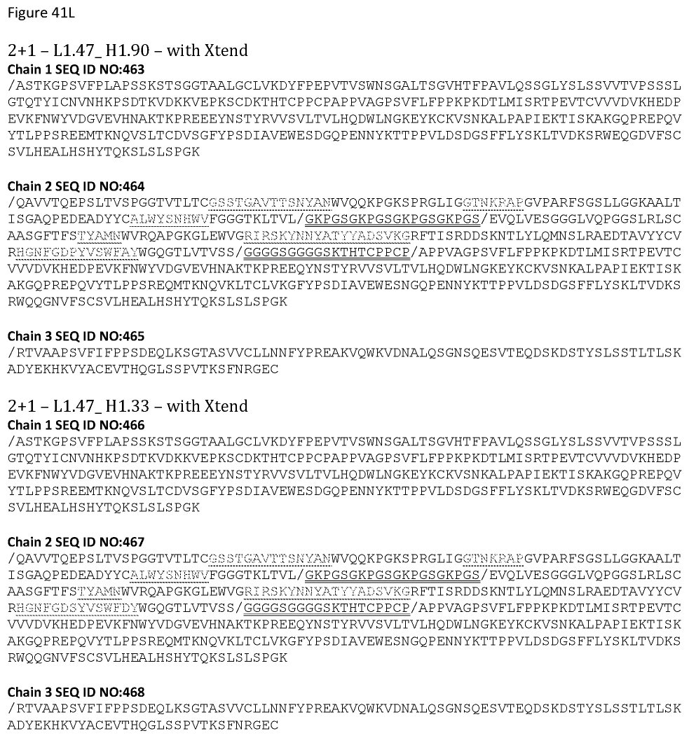

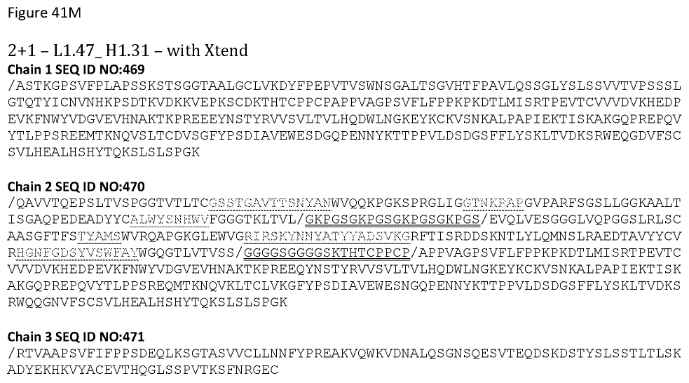

Accordingly, there are a number of useful Fc substitutions that can be made to alter binding to one or more of the FcγR receptors. Substitutions that result in increased binding as well as decreased binding can be useful. For example, it is known that increased binding to FcγRIIIa generally results in increased ADCC (antibody dependent cell-mediated cytotoxicity; the cell-mediated reaction wherein nonspecific cytotoxic cells that express FcγRs recognize bound antibody on a target cell and subsequently cause lysis of the target cell). Similarly, decreased binding to FcγRIIb (an inhibitory receptor) can be beneficial as well in some circumstances. Amino acid substitutions that find use in the present invention include those listed in U.S. Ser. Nos. 11/124,620 (particularly FIG. 41), 11/174,287, 11/396,495, 11/538,406, all of which are expressly incorporated herein by reference in their entirety and specifically for the variants disclosed therein. Particular variants that find use include, but are not limited to, 236A, 239D, 239E, 332E, 332D, 239D/332E, 267D, 267E, 328F, 267E/328F, 236A/332E, 239D/332E/330Y, 239D/332E/330L, 243A, 243L, 264A, 264V and 299T.

In addition, there are additional Fc substitutions that find use in increased binding to the FcRn receptor and increased serum half-life, as specifically disclosed in U.S. Ser. No. 12/341,769, hereby incorporated by reference in its entirety, including, but not limited to, 434S, 434A, 428L, 308F, 2591, 428L/434S, 2591/308F, 4361/428L, 4361 or V/434S, 436V/428L and 2591/308F/428L.

### Ablation Variants

Similarly, another category of functional variants are “FcγR ablation variants” or “Fc knock out (FcKO or KO)” variants. In these embodiments, for some therapeutic applications, it is desirable to reduce or remove the normal binding of the Fc domain to one or more or all of the Fcγ receptors (e.g. FcγR1, FcγRIIa, FcγRIIb, FcγRIIIa, etc.) to avoid additional mechanisms of action. That is, for example, in many embodiments, particularly in the use of bispecific antibodies that bind CD3 monovalently it is generally desirable to ablate FcγRIIIa binding to eliminate or significantly reduce ADCC activity. wherein one of the Fc domains comprises one or more Fcγ receptor ablation variants. These ablation variants are depicted in FIG. 3, and each can be independently and optionally included or excluded, with preferred aspects utilizing ablation variants selected from the group consisting of G236R/L328R, E233P/L234V/L235A/G236del/S239K, E233P/L234V/L235A/G236del/S267K, E233P/L234V/L235A/G236del/S239K/A327G, E233P/L234V/L235A/G236del/S267K/A327G and E233P/L234V/L235A/G236del. It should be noted that the ablation variants referenced herein ablate FcγR binding but generally not FcRn binding.

As is known in the art, the Fc domain of human IgG1 has the highest binding to the Fcγ receptors, and thus ablation variants can be used when the constant domain (or Fc domain) in the backbone of the heterodimeric antibody is IgG1. Alternatively, or in addition to ablation variants in an IgG1 background, mutations at the glycosylation position 297 (generally to A or S) can significantly ablate binding to FcγRIIIa, for example. Human IgG2 and IgG4 have naturally reduced binding to the Fcγ receptors, and thus those backbones can be used with or without the ablation variants.

### Combination of Heterodimeric and Fc Variants

As will be appreciated by those in the art, all of the recited heterodimerization variants (including skew and/or pI variants) can be optionally and independently combined in any way, as long as they retain their “strandedness” or “monomer partition”. In addition, all of these variants can be combined into any of the heterodimerization formats.

In the case of pI variants, while embodiments finding particular use are shown in the Figures, other combinations can be generated, following the basic rule of altering the pI difference between two monomers to facilitate purification.

In addition, any of the heterodimerization variants, skew and pI, are also independently and optionally combined with Fc ablation variants, Fc variants, FcRn variants, as generally outlined herein.

### H. Useful Formats of the Invention

As will be appreciated by those in the art and discussed more fully below, the heterodimeric fusion proteins of the present invention can take on a wide variety of configurations, as are generally depicted in FIGS. 13 and 42. Some figures depict “single ended” configurations, where there is one type of specificity on one “arm” of the molecule and a different specificity on the other “arm”. Other figures depict “dual ended” configurations, where there is at least one type of specificity at the “top” of the molecule and one or more different specificities at the “bottom” of the molecule. Thus, the present invention is directed to novel immunoglobulin compositions that co-engage a different first and a second antigen.

As will be appreciated by those in the art, the heterodimeric formats of the invention can have different valencies as well as be bispecific. That is, heterodimeric antibodies of the invention can be bivalent and bispecific, wherein one target tumor antigen (e.g. CD3) is bound by one binding domain and the other target tumor antigen (e.g. CLDN18.2) is bound by a second binding domain. The heterodimeric antibodies can also be trivalent and bispecific, wherein the first antigen is bound by two binding domains and the second antigen by a second binding domain. As is outlined herein, when CD3 is one of the target antigens, it is preferable that the CD3 is bound only monovalently, to reduce potential side effects.

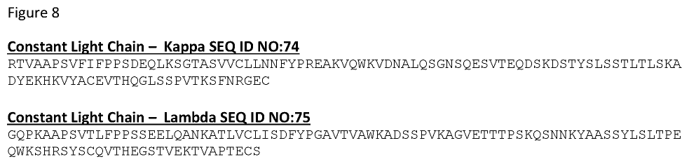

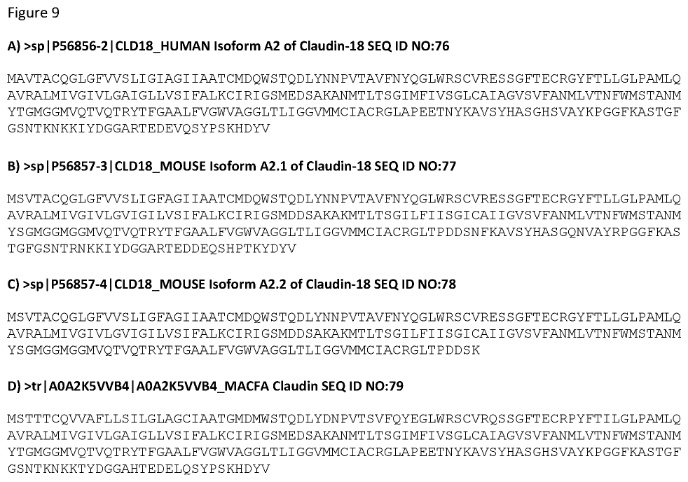

The present invention utilizes anti-CD3 antigen binding domains in combination with anti-CLDN18.2 binding domains. As will be appreciated by those in the art, any collection of anti-CD3 CDRs, anti-CD3 variable light and variable heavy domains, Fabs and scFvs as depicted in any of the Figures can be used. Similarly, any of the anti-CLDN18.2 antigen binding domains can be used, whether CDRs, variable light and variable heavy domains, Fabs and scFvs as depicted in any of the Figures (e.g., FIGS. 8 and 10) can be used, optionally and independently combined in any combination.

### 1+1 Fab-scFv-Fc Format

One heterodimeric scaffold that finds particular use in the present invention is the “1+1 Fab-scFv-Fc” format of FIG. 13A and FIG. 42A. In this embodiment, one heavy chain of the antibody contains an single chain Fv (“scFv”, as defined below) and the other heavy chain is a “regular” Fab format, comprising a variable heavy chain and a light chain. This structure is sometimes referred to in previous related filings as “triple F” format (scFv-Fab-Fc) or the “bottle-opener” format, due to a rough visual similarity to a bottle-opener. The two chains are brought together by the use of amino acid variants in the constant regions (e.g., the Fc domain, the CH1 domain and/or the hinge region) that promote the formation of heterodimeric antibodies as is described more fully below.

There are several distinct advantages to the present “1+1 Fab-scFv-Fc” format. As is known in the art, antibody analogs relying on two scFv constructs often have stability and aggregation problems, which can be alleviated in the present invention by the addition of a “regular” heavy and light chain pairing. In addition, as opposed to formats that rely on two heavy chains and two light chains, there is no issue with the incorrect pairing of heavy and light chains (e.g. heavy 1 pairing with light 2, etc.).

Many of the embodiments outlined herein rely in general on the bottle opener format that comprises a first monomer comprising an scFv, comprising a variable heavy and a variable light domain, covalently attached using an scFv linker (charged, in many but not all instances), where the scFv is covalently attached to the N-terminus of a first Fc domain usually through a domain linker (which, as outlined herein can either be un-charged or charged). The second monomer of the bottle opener format is a heavy chain, and the composition further comprises a light chain.

In general, in many preferred embodiments, the scFv is the domain that binds to the CD3, and the Fab forms a CLDN18.2 binding domain.

In addition, the Fc domains of the invention generally comprise skew variants (e.g. a set of amino acid substitutions as shown in FIGS. 1 and 4, with particularly useful skew variants being selected from the group consisting of S364K/E357Q:L368D/K370S; L368D/K370S:S364K; L368E/K370S:S364K; T411T/E360E/Q362E:D401K; L368D/K370S:S364K/E357L and K370S:S364K/E357Q), optionally ablation variants (including those shown in FIG. 3), optionally charged scFv linkers (including those shown in FIG. 5) and the heavy chain comprises pI variants (including those shown in FIG. 2).

In some embodiments, the bottle opener format includes skew variants, pI variants, and ablation variants. Accordingly, some embodiments include bottle opener formats that comprise: a) a first monomer (the “scFv monomer”) that comprises a charged scFv linker (with the +H sequence of FIG. 5 being preferred in some embodiments), the skew variants S364K/E357Q, the ablation variants E233P/L234V/L235A/G236del/S267K, and an Fv that binds to CD3 as outlined herein; b) a second monomer (the “Fab monomer”) that comprises the skew variants L368D/K370S, the pI variants N208D/Q295E/N384D/Q418E/N421D, the ablation variants E233P/L234V/L235A/G236del/S267K, and a variable heavy domain that, with the variable light domain, makes up an Fv that binds to CLDN18.2 as outlined herein; and c) a light chain.

Exemplary variable heavy and light domains of the scFv that binds to CD3 are included in FIG. 12. Exemplary variable heavy and light domains of the Fv that binds to CLDN18.2 are included in FIG. 10.

In some embodiments, the bottle opener format includes skew variants, pI variants, ablation variants and FcRn variants. Accordingly, some embodiments include bottle opener formats that comprise: a) a first monomer (the “scFv monomer”) that comprises a charged scFv linker (with the +H sequence of FIG. 2 being preferred in some embodiments), the skew variants S364K/E357Q, the ablation variants E233P/L234V/L235A/G236del/S267K, the FcRn variants M428L/N434S and an Fv that binds to CD3 as outlined herein; b) a second monomer (the “Fab monomer”) that comprises the skew variants L368D/K370S, the pI variants N208D/Q295E/N384D/Q418E/N421D, the ablation variants E233P/L234V/L235A/G236del/S267K, the FcRn variants M428L/N434S, and a variable heavy domain that, with the variable light domain, makes up an Fv that binds to CLDN18.2 as outlined herein; and c) a light chain.

Exemplary variable heavy and light domains of scFvs that bind to CD3 are included in FIG. 12. Exemplary variable heavy and light domains of the Fv that binds to CLDN18.2 are included in FIG. 10.

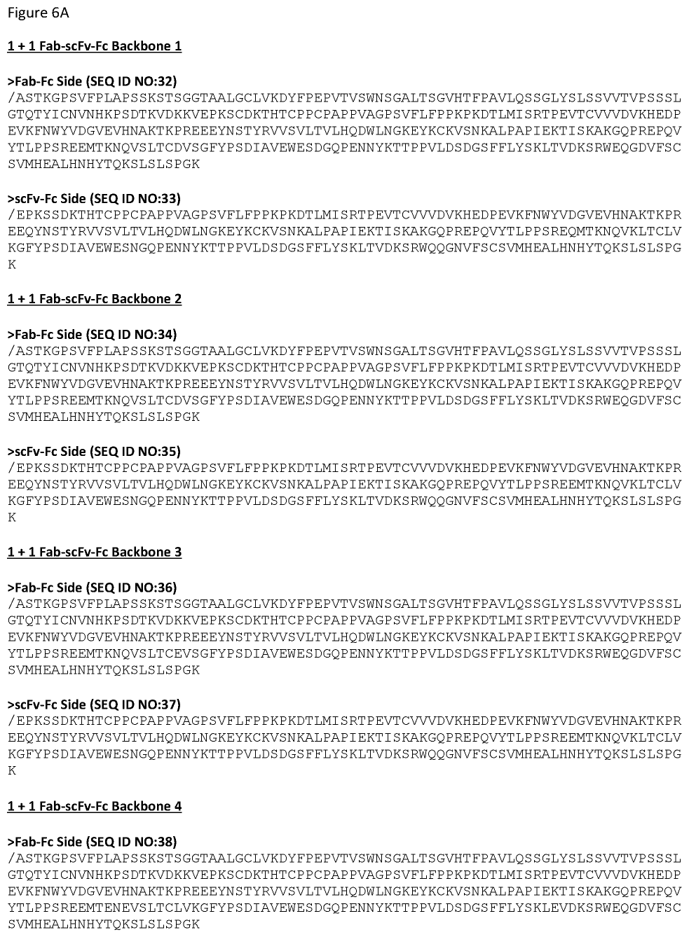

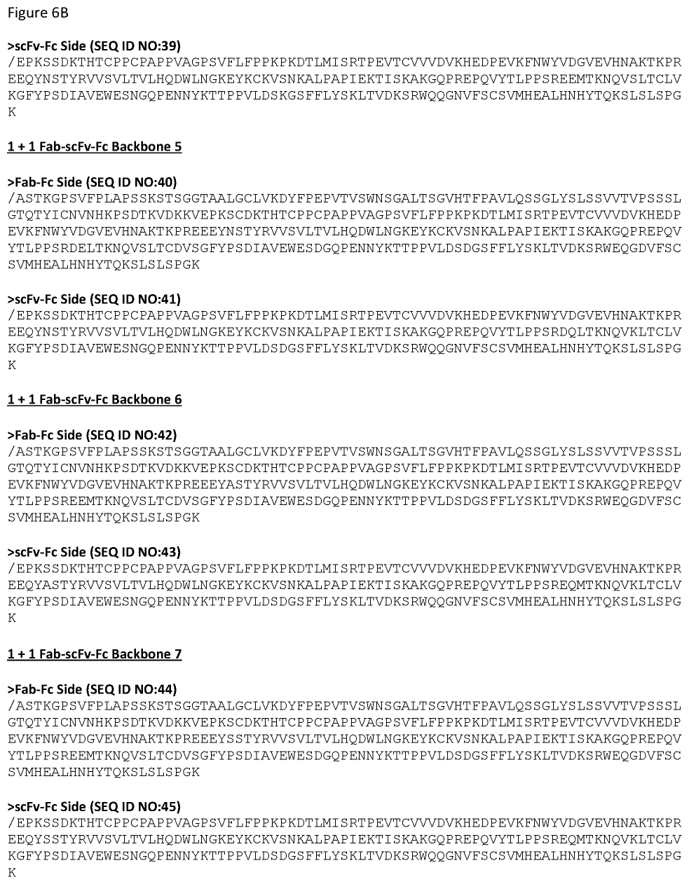

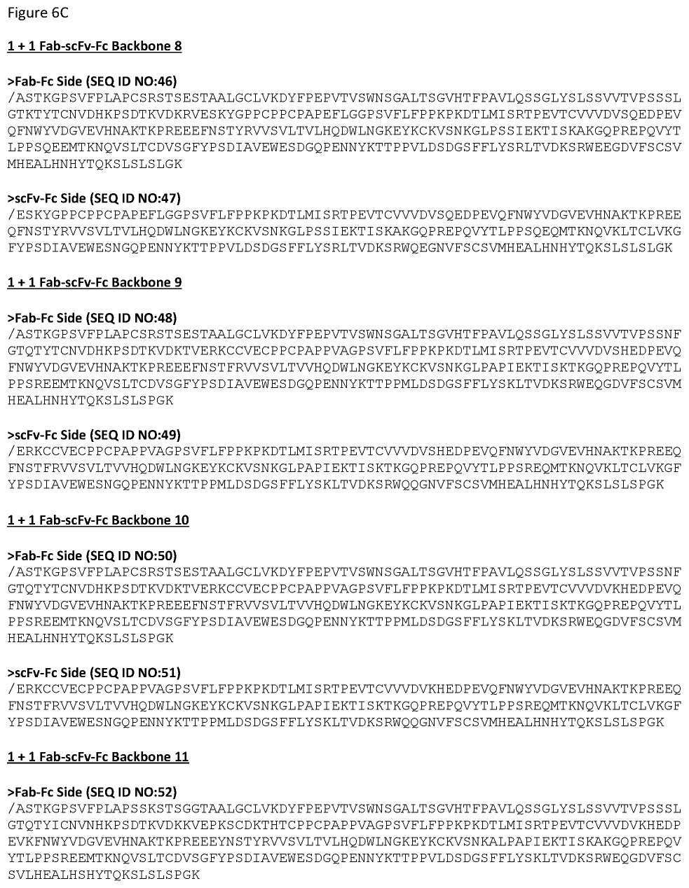

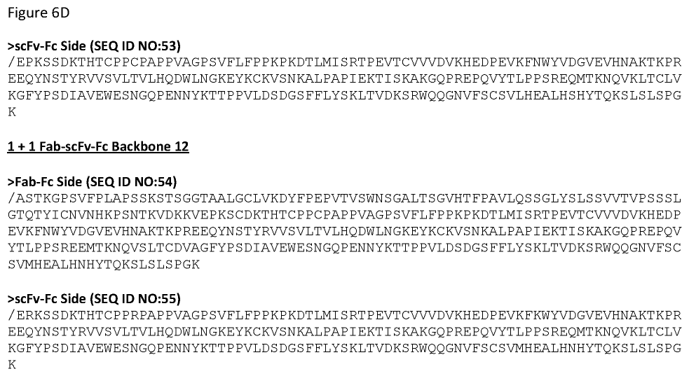

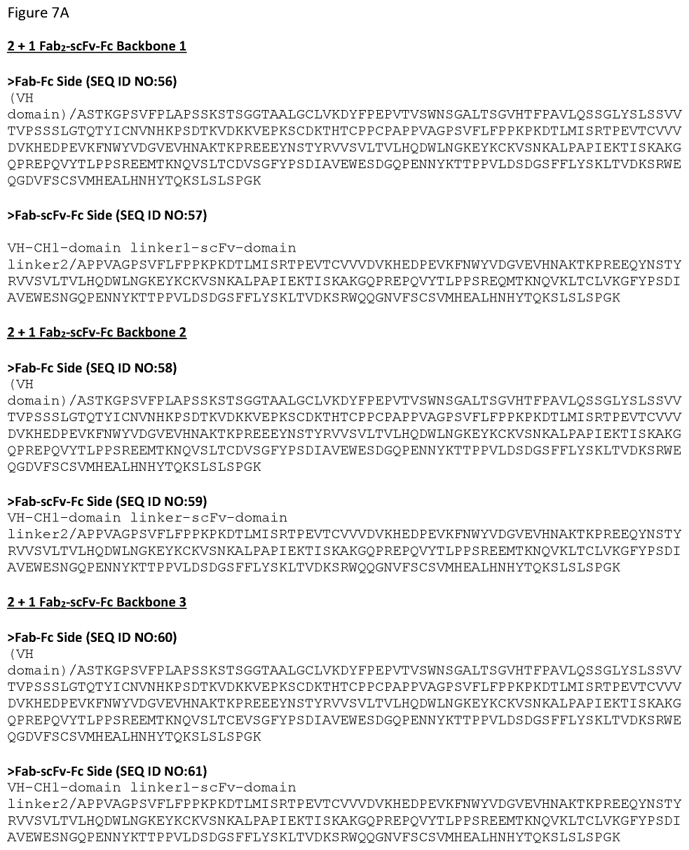

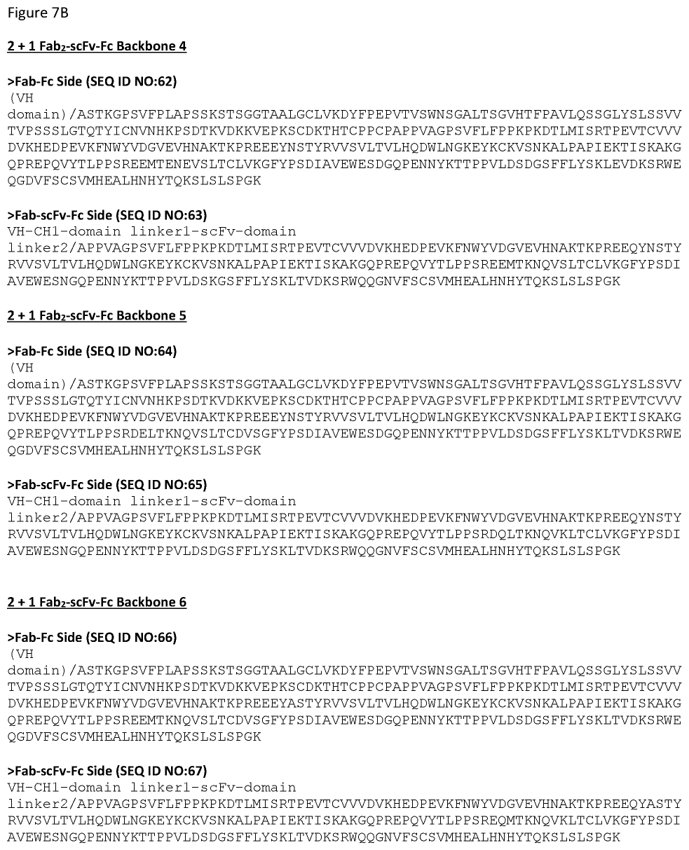

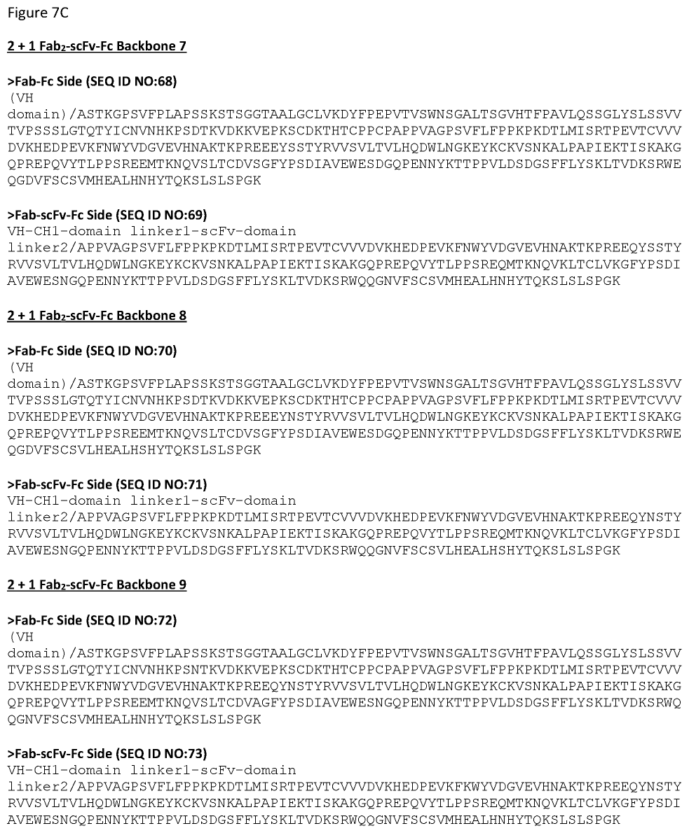

FIG. 6 shows some exemplary bottle opener “backbone” sequences that are missing the Fv sequences that can be used in the present invention. In some embodiments, any of the VH and VL sequences depicted herein (including all VH and VL sequences depicted in the Figures and Sequence Listings, including those directed to CLDN18.2) can be added to the bottle opener backbone formats of FIG. 6 as the “Fab side”, using any of the anti-CD3 scFv sequences shown in the Figures and Sequence Listings.

For bottle opener backbone 1 from FIG. 6, (optionally including the 428L/434S variants), CD binding domain sequences finding particular use in these embodiments include, but are not limited to, CD3 binding domain anti-CD3 H1.30_L1.47, anti-CD3 H1.32_L1.47, anti-CD3 H1.89_L1.47, anti-CD3 H1.90_L1.47, anti-CD3 H1.33_L1.47 and anti-CD3 H1.31_L1.47, as well as those depicted in FIG. 12, attached as the scFv side of the backbones shown in FIG. 6.

Particularly useful CLDN18.2 and CD3 sequence combinations for use with bottle opener backbone 1 from FIG. 6, (optionally including the 428L/434S variants), are disclosed in FIG. 31.

mAb-Fv

One heterodimeric scaffold that finds particular use in the present invention is the mAb-Fv format shown in FIG. 42G. In this embodiment, the format relies on the use of a C-terminal attachment of an “extra” variable heavy domain to one monomer and the C-terminal attachment of an “extra” variable light domain to the other monomer, thus forming a third antigen binding domain, wherein the Fab portions of the two monomers bind a CLDN18.2 and the “extra” scFv domain binds CD3.

In this embodiment, the first monomer comprises a first heavy chain, comprising a first variable heavy domain and a first constant heavy domain comprising a first Fc domain, with a first variable light domain covalently attached to the C-terminus of the first Fc domain using a domain linker (VH1-CH1-hinge-CH2-CH3-[optional linker]-VL2). The second monomer comprises a second variable heavy domain of the second constant heavy domain comprising a second Fc domain, and a third variable heavy domain covalently attached to the C-terminus of the second Fc domain using a domain linker (VH1-CH1-hinge-CH2-CH3-[optional linker]-VH2. The two C-terminally attached variable domains make up a Fv that binds CD3 (as it is less preferred to have bivalent CD3 binding). This embodiment further utilizes a common light chain comprising a variable light domain and a constant light domain that associates with the heavy chains to form two identical Fabs that bind a CLDN18.2. As for many of the embodiments herein, these constructs include skew variants, pI variants, ablation variants, additional Fc variants, etc. as desired and described herein.

The present invention provides mAb-Fv formats where the CD3 binding domain sequences are as shown in FIG. 12. The present invention provides mAb-Fv formats wherein the CLDN18.2 binding domain sequences are as shown in FIG. 10.

In addition, the Fc domains of the mAb-Fv format comprise skew variants (e.g. a set of amino acid substitutions as shown in FIGS. 1 and 4, with particularly useful skew variants being selected from the group consisting of S364K/E357Q:L368D/K370S; L368D/K370S:S364K; L368E/K370S:S364K; T411T/E360E/Q362E:D401K; L368D/K370S:S364K/E357L, K370S:S364K/E357Q, T366S/L368A/Y407V:T366W and T366S/L368A/Y407V/Y349C:T366W/S354C), optionally ablation variants (including those shown in FIG. 3), optionally charged scFv linkers (including those shown in FIG. 5) and the heavy chain comprises pI variants (including those shown in FIG. 2).

In some embodiments, the mAb-Fv format includes skew variants, pI variants, and ablation variants. Accordingly, some embodiments include mAb-Fv formats that comprise: a) a first monomer that comprises the skew variants S364K/E357Q, the ablation variants E233P/L234V/L235A/G236del/S267K, and a first variable heavy domain that, with the first variable light domain of the light chain, makes up an Fv that binds to CLDN18.2, and a second variable heavy domain; b) a second monomer that comprises the skew variants L368D/K370S, the pI variants N208D/Q295E/N384D/ Q418E/N421D, the ablation variants E233P/L234V/L235A/G236del/S267K, and a first variable heavy domain that, with the first variable light domain, makes up the Fv that binds to CLDN18.2 as outlined herein, and a second variable light chain, that together with the second variable heavy domain forms an Fv (ABD) that binds to CD3; and c) a light chain comprising a first variable light domain and a constant light domain.

In some embodiments, the mAb-Fv format includes skew variants, pI variants, ablation variants and FcRn variants. Accordingly, some embodiments include mAb-Fv formats that comprise: a) a first monomer that comprises the skew variants S364K/E357Q, the ablation variants E233P/L234V/L235A/G236del/S267K, the FcRn variants M428L/N434S and a first variable heavy domain that, with the first variable light domain of the light chain, makes up an Fv that binds to CLDN18.2, and a second variable heavy domain; b) a second monomer that comprises the skew variants L368D/K370S, the pI variants N208D/Q295E/N384D/Q418E/N421D, the ablation variants E233P/L234V/L235A/G236del/S267K, the FcRn variants M428L/N434S and a first variable heavy domain that, with the first variable light domain, makes up the Fv that binds to CLDN18.2 as outlined herein, and a second variable light chain, that together with the second variable heavy domain of the first monomer forms an Fv (ABD) that binds CD3; and c) a light chain comprising a first variable light domain and a constant light domain.

mAb-scFv

One heterodimeric scaffold that finds particular use in the present invention is the mAb-scFv format shown in FIG. 42H. In this embodiment, the format relies on the use of a C-terminal attachment of a scFv to one of the monomers, thus forming a third antigen binding domain, wherein the Fab portions of the two monomers bind CLDN18.2 and the “extra” scFv domain binds CD3. Thus, the first monomer comprises a first heavy chain (comprising a variable heavy domain and a constant domain), with a C-terminally covalently attached scFv comprising a scFv variable light domain, an scFv linker and a scFv variable heavy domain in either orientation (VH1-CH1-hinge-CH2-CH3-[optional linker]-VH2-scFv linker-VL2 or VH1-CH1-hinge-CH2-CH3-[optional linker]-VL2-scFv linker-VH2). This embodiment further utilizes a common light chain comprising a variable light domain and a constant light domain, that associates with the heavy chains to form two identical Fabs that bind CLDN18.2. As for many of the embodiments herein, these constructs include skew variants, pI variants, ablation variants, additional Fc variants, etc. as desired and described herein.

The present invention provides mAb-scFv formats where the CD binding domain sequences are as shown in FIG. 12 and the CLDN18.2 binding domain sequences are as shown in FIG. 10.

In addition, the Fc domains of the mAb-scFv format comprise skew variants (e.g. a set of amino acid substitutions as shown in FIG. 1, with particularly useful skew variants being selected from the group consisting of S364K/E357Q:L368D/K370S; L368D/K370S:S364K; L368E/K370S:S364K; T411T/E360E/Q362E:D401K; L368D/K370S:S364K/E357L, K370S:S364K/E357Q, T366S/L368A/Y407V:T366W and T366S/L368A/Y407V/Y349C:T366W/S354C), optionally ablation variants (including those shown in FIG. 3), optionally charged scFv linkers (including those shown in FIG. 5) and the heavy chain comprises pI variants (including those shown in FIG. 2).

In some embodiments, the mAb-scFv format includes skew variants, pI variants, and ablation variants. Accordingly, some embodiments include mAb-scFv formats that comprise: a) a first monomer that comprises the skew variants S364K/E357Q, the ablation variants E233P/L234V/L235A/G236del/S267K, and a variable heavy domain that, with the variable light domain of the common light chain, makes up an Fv that binds to CLDN18.2 as outlined herein, and a scFv domain that binds to CD3; b) a second monomer that comprises the skew variants L368D/K370S, the pI variants N208D/Q295E/N384D/Q418E/N421D, the ablation variants E233P/L234V/L235A/G236del/S267K, and a variable heavy domain that, with the variable light domain of the common light chain, makes up an Fv that binds to CLDN18.2 as outlined herein; and c) a common light chain comprising a variable light domain and a constant light domain.

In some embodiments, the mAb-scFv format includes skew variants, pI variants, ablation variants and FcRn variants. Accordingly, some embodiments include mAb-scFv formats that comprise: a) a first monomer that comprises the skew variants S364K/E357Q, the ablation variants E233P/L234V/L235A/G236del/S267K, the FcRn variants M428L/N434S and a variable heavy domain that, with the variable light domain of the common light chain, makes up an Fv that binds to CLDN18.2 as outlined herein, and a scFv domain that binds to CD3; b) a second monomer that comprises the skew variants L368D/K370S, the pI variants N208D/Q295E/N384D/Q418E/N421D, the ablation variants E233P/L234V/L235A/G236del/S267K, the FcRn variants M428L/N434S and a variable heavy domain that, with the variable light domain of the common light chain, makes up an Fv that binds to CLDN18.2 as outlined herein; and c) a common light chain comprising a variable light domain and a constant light domain.

### 2+1 Fab₂-scFv-Fc Format

One heterodimeric scaffold that finds particular use in the present invention is the “2+1 Fab₂-scFv-Fc” format (also referred to in previous related filings as “Central-scFv format”) shown in FIG. 13B and FIG. 42F. In this embodiment, the format relies on the use of an inserted scFv domain thus forming a third antigen binding domain, wherein the Fab portions of the two monomers bind CLDN18.2 and the “extra” scFv domain binds CD3. The scFv domain is inserted between the Fc domain and the CH1-Fv region of one of the monomers, thus providing a third antigen binding domain.

In this embodiment, one monomer comprises a first heavy chain comprising a first variable heavy domain, a CH1 domain (and optional hinge) and Fc domain, with a scFv comprising a scFv variable light domain, an scFv linker and a scFv variable heavy domain. The scFv is covalently attached between the C-terminus of the CH1 domain of the heavy constant domain and the N-terminus of the first Fc domain using optional domain linkers (VH1-CH1-[optional linker]-VH2-scFv linker-VL2-[optional linker including the hinge]-CH2-CH3, or the opposite orientation for the scFv, VH1-CH1-[optional linker]-VL2-scFv linker-VH2-[optional linker including the hinge]-CH2-CH3). The other monomer is a standard Fab side. This embodiment further utilizes a common light chain comprising a variable light domain and a constant light domain, that associates with the heavy chains to form two identical Fabs that bind CLDN18.2. As for many of the embodiments herein, these constructs include skew variants, pI variants, ablation variants, additional Fc variants, etc. as desired and described herein.

The present invention provides “2+1 Fab₂-scFv-Fc” formats where the CD3 binding domain sequences are as shown in FIG. 12 and the anti-CLDN18.2 sequences are as shown in FIG. 10.

In addition, the Fc domains of the central scFv format comprise skew variants (e.g. a set of amino acid substitutions as shown in FIG. 1, with particularly useful skew variants being selected from the group consisting of S364K/E357Q:L368D/K370S; L368D/K370S:S364K; L368E/K370S:S364K; T411T/E360E/Q362E:D401K; L368D/K370S:S364K/E357L, K370S:S364K/E357Q, T366S/L368A/Y407V:T366W and T366S/L368A/Y407V/Y349C:T366W/S354C), optionally ablation variants (including those shown in FIG. 3), optionally charged scFv linkers (including those shown in FIG. 5) and the heavy chain comprises pI variants (including those shown in FIG. 2).

In some embodiments, the central-scFv format includes skew variants, pI variants, and ablation variants. Accordingly, some embodiments include central scFv formats that comprise: a) a first monomer that comprises the skew variants S364K/E357Q, the ablation variants E233P/L234V/L235A/G236del/S267K, and a variable heavy domain that, with the variable light domain of the light chain, makes up an Fv that binds to CLDN18.2 as outlined herein, and an scFv domain that binds to CD3; b) a second monomer that comprises the skew variants L368D/K370S, the pI variants N208D/Q295E/N384D/Q418E/N421D, the ablation variants E233P/L234V/L235A/G236del/S267K, and a variable heavy domain that, with variable light domain of the light chain, makes up an Fv that binds to CLDN18.2 as outlined herein; and c) a light chain comprising a variable light domain and a constant light domain.

In some embodiments, the central-scFv format includes skew variants, pI variants, ablation variants and FcRn variants. Accordingly, some embodiments include central scFv formats that comprise: a) a first monomer that comprises the skew variants S364K/E357Q, the ablation variants E233P/L234V/L235A/G236del/S267K, the FcRn variants M428L/N434S and a variable heavy domain that, with the variable light domain of the light chain, makes up an Fv that binds to CLDN18.2 as outlined herein, and an scFv domain that binds to CD3; b) a second monomer that comprises the skew variants L368D/K370S, the pI variants N208D/Q295E/N384D/Q418E/N421D, the ablation variants E233P/L234V/L235A/G236del/S267K, the FcRn variants M428L/N434S and a variable heavy domain that, with variable light domain of the light chain, makes up an Fv that binds to CLDN18.2 as outlined herein; and c) a light chain comprising a variable light domain and a constant light domain.

### Central-Fv

One heterodimeric scaffold that finds particular use in the present invention is the Central-Fv format shown in FIG. 42I. In this embodiment, the format relies on the use of an inserted Fv domain (i.e., the central Fv domain) thus forming a third antigen binding domain, wherein the Fab portions of the two monomers bind a CLDN18.2 and the “central Fv” domain binds CD3. The scFv domain is inserted between the Fc domain and the CH1-Fv region of the monomers, thus providing a third antigen binding domain, wherein each monomer contains a component of the scFv (e.g. one monomer comprises a variable heavy domain and the other a variable light domain).

In this embodiment, one monomer comprises a first heavy chain comprising a first variable heavy domain, a CH1 domain, and Fc domain and an additional variable light domain. The light domain is covalently attached between the C-terminus of the CH1 domain of the heavy constant domain and the N-terminus of the first Fc domain using domain linkers (VH1-CH1-[optional linker]-VL2-hinge-CH2-CH3). The other monomer comprises a first heavy chain comprising a first variable heavy domain, a CH1 domain and Fc domain and an additional variable heavy domain (VH1-CH1-[optional linker]-VH2-hinge-CH2-CH3). The light domain is covalently attached between the C-terminus of the CH1 domain of the heavy constant domain and the N-terminus of the first Fc domain using domain linkers.

This embodiment further utilizes a common light chain comprising a variable light domain and a constant light domain, that associates with the heavy chains to form two identical Fabs that bind a CLDN18.2. As for many of the embodiments herein, these constructs include skew variants, pI variants, ablation variants, additional Fc variants, etc. as desired and described herein.

The present invention provides central-Fv formats where the CD3 binding domain sequences are as shown in FIG. 12 and the CLDN18.2 binding domain sequences are as shown in FIG. 10.

For central-Fv formats, CD3 binding domain sequences finding particular use in these embodiments include, but are not limited to, anti-CD3 H1.30_L1.47, anti-CD3 H1.32_L1.47, anti-CD3 H1.89_L1.47, anti-CD3 H1.90_L1.47, anti-CD3 H1.33_L1.47 and anti-CD3 H1.31_L1.47 as depicted in FIG. 12.

### One Armed Central-scFv

One heterodimeric scaffold that finds particular use in the present invention is the one armed central-scFv format shown in FIG. 42C. In this embodiment, one monomer comprises just an Fc domain, while the other monomer uses an inserted scFv domain thus forming the second antigen binding domain. In this format, either the Fab portion binds a CLDN18.2 and the scFv binds CD3 or vice versa. The scFv domain is inserted between the Fc domain and the CH1-Fv region of one of the monomers.

In this embodiment, one monomer comprises a first heavy chain comprising a first variable heavy domain, a CH1 domain and Fc domain, with a scFv comprising a scFv variable light domain, an scFv linker and a scFv variable heavy domain. The scFv is covalently attached between the C-terminus of the CH1 domain of the heavy constant domain and the N-terminus of the first Fc domain using domain linkers. The second monomer comprises an Fc domain. This embodiment further utilizes a light chain comprising a variable light domain and a constant light domain, that associates with the heavy chain to form a Fab. As for many of the embodiments herein, these constructs include skew variants, pI variants, ablation variants, additional Fc variants, etc. as desired and described herein.

The present invention provides central-Fv formats where the CD3 binding domain sequences are as shown in FIG. 12 and the CLDN18.2 binding domain sequences are as shown in FIG. 10.

In addition, the Fc domains of the one armed central-scFv format generally include skew variants (e.g. a set of amino acid substitutions as shown in FIG. 1, with particularly useful skew variants being selected from the group consisting of S364K/E357Q:L368D/K370S; L368D/K370S:S364K; L368E/K370S:S364K; T411T/E360E/Q362E:D401K; L368D/K370S:S364K/E357L, K370S:S364K/E357Q, T366S/L368A/Y407V:T366W and T366S/L368A/Y407V/Y349C:T366W/S354C), optionally ablation variants (including those shown in FIG. 3), optionally charged scFv linkers (including those shown in FIG. 5) and the heavy chain comprises pI variants (including those shown in FIG. 2).

In some embodiments, the one armed central-scFv format includes skew variants, pI variants, and ablation variants. Accordingly, some embodiments of the one armed central-scFv formats comprise: a) a first monomer that comprises the skew variants S364K/E357Q, the ablation variants E233P/L234V/L235A/G236del/S267K, and a variable heavy domain that, with the variable light domain of the light chain, makes up an Fv that binds to CLDN18.2 as outlined herein, and a scFv domain that binds to CD3; b) a second monomer that includes an Fc domain having the skew variants L368D/K370S, the pI variants N208D/Q295E/N384D/ Q418E/N421D, the ablation variants E233P/L234V/L235A/G236del/S267K; and c) a light chain comprising a variable light domain and a constant light domain.

In some embodiments, the one armed central-scFv format includes skew variants, pI variants, ablation variants and FcRn variants. Accordingly, some embodiments of the one armed central-scFv formats comprise: a) a first monomer that comprises the skew variants S364K/E357Q, the ablation variants E233P/L234V/L235A/G236del/S267K, the FcRn variants M428L/N434S and a variable heavy domain that, with the variable light domain of the light chain, makes up an Fv that binds to CLDN18.2 as outlined herein, and a scFv domain that binds to CD3; b) a second monomer that includes an Fc domain having the skew variants L368D/K370S, the pI variants N208D/Q295E/N384D/Q418E/N421D, the ablation variants E233P/L234V/L235A/G236del/S267K, and the FcRn variants M428L/N434S; and c) a light chain comprising a variable light domain and a constant light domain.

For one armed central-scFv formats, CD3 binding domain sequences finding particular use include, but are not limited to, anti-CD3 H1.30_L1.47, anti-CD3 H1.32_L1.47, anti-CD3 H1.89_L1.47, anti-CD3 H1.90_L1.47, anti-CD3 H1.33_L1.47 and anti-CD3 H1.31_L1.47, as depicted in FIG. 12.

One Armed scFv-mAb

One heterodimeric scaffold that finds particular use in the present invention is the one armed scFv-mAb format shown in FIG. 42D. In this embodiment, one monomer comprises just an Fc domain, while the other monomer uses a scFv domain attached at the N-terminus of the heavy chain, generally through the use of a linker: VH-scFv linker-VL-[optional domain linker]-CH1-hinge-CH2-CH3 or (in the opposite orientation) VL-scFv linker-VH-[optional domain linker]-CH1-hinge-CH2-CH3. In this format, the Fab portions each bind CLDN18.2 and the scFv binds CD3. This embodiment further utilizes a light chain comprising a variable light domain and a constant light domain, that associates with the heavy chain to form a Fab. As for many of the embodiments herein, these constructs include skew variants, pI variants, ablation variants, additional Fc variants, etc. as desired and described herein.

The present invention provides one armed scFv-mAb formats where the CD3 binding domain sequences are as shown in FIG. 12 and wherein the CLDN18.2 binding domain sequences are as shown in FIG. 10.

In addition, the Fc domains of the one armed scFv-mAb format generally include skew variants (e.g. a set of amino acid substitutions as shown in FIGS. 1 and 4, with particularly useful skew variants being selected from the group consisting of S364K/E357Q:L368D/K370S; L368D/K370S:S364K; L368E/K370S:S364K; T411T/E360E/Q362E:D401K; L368D/K370S:S364K/E357L, K370S:S364K/E357Q, T366S/L368A/Y407V:T366W and T366S/L368A/Y407V/Y349C:T366W/S354C), optionally ablation variants (including those shown in FIG. 3), optionally charged scFv linkers (including those shown in FIG. 5) and the heavy chain comprises pI variants (including those shown in FIG. 2).

In some embodiments, the one armed scFv-mAb format includes skew variants, pI variants, and ablation variants. Accordingly, some embodiments of the one armed scFv-mAb formats comprise: a) a first monomer that comprises the skew variants S364K/E357Q, the ablation variants E233P/L234V/L235A/G236del/S267K, and a variable heavy domain that, with the variable light domain of the light chain, makes up an Fv that binds to CLDN18.2 as outlined herein, and a scFv domain that binds to CD3; b) a second monomer that includes an Fc domain having the skew variants L368D/K370S, the pI variants N208D/Q295E/N384D/Q418E/N421D, the ablation variants E233P/L234V/L235A/G236del/S267K; and c) a light chain comprising a variable light domain and a constant light domain.

In some embodiments, the one armed scFv-mAb format includes skew variants, pI variants, ablation variants and FcRn variants. Accordingly, some embodiments one armed scFv-mAb formats comprise: a) a first monomer that comprises the skew variants S364K/E357Q, the ablation variants E233P/L234V/L235A/G236del/S267K, the FcRn variants M428L/N434S and a variable heavy domain that, with the variable light domain of the light chain, makes up an Fv that binds to CLDN18.2 as outlined herein, and a scFv domain that binds to CD3; b) a second monomer that includes an Fc domain having the skew variants L368D/K370S, the pI variants N208D/Q295E/N384D/Q418E/N421D, the ablation variants E233P/L234V/L235A/G236del/S267K, and the FcRn variants M428L/N434S; and c) a light chain comprising a variable light domain and a constant light domain.

For one armed scFv-mAb formats, CD3 binding domain sequences finding particular use include, but are not limited to, anti-CD3 H1.30_L1.47, anti-CD3 H1.32_L1.47, anti-CD3 H1.89_L1.47, anti-CD3 H1.90_L1.47, anti-CD3 H1.33_L1.47 and anti-CD3 H1.31_L1.47, as depicted in FIG. 12.

scFv-mAb

One heterodimeric scaffold that finds particular use in the present invention is the mAb-scFv format shown in FIG. 42E. In this embodiment, the format relies on the use of a N-terminal attachment of a scFv to one of the monomers, thus forming a third antigen binding domain, wherein the Fab portions of the two monomers bind CLDN18.2 and the “extra” scFv domain binds CD3.

In this embodiment, the first monomer comprises a first heavy chain (comprising a variable heavy domain and a constant domain), with a N-terminally covalently attached scFv comprising a scFv variable light domain, an scFv linker and a scFv variable heavy domain in either orientation ((VH1-scFv linker-VL1-[optional domain linker]-VH2-CH1-hinge-CH2-CH3) or (with the scFv in the opposite orientation) ((VL1-scFv linker-VH1-[optional domain linker]-VH2-CH1-hinge-CH2-CH3)). This embodiment further utilizes a common light chain comprising a variable light domain and a constant light domain that associates with the heavy chains to form two identical Fabs that bind CLDN18.2. As for many of the embodiments herein, these constructs include skew variants, pI variants, ablation variants, additional Fc variants, etc. as desired and described herein.

The present invention provides scFv-mAb formats where the CD3 binding domain sequences are as shown in FIG. 12 and wherein the CLDN18.2 binding domain sequences are as shown in FIG. 10.

In addition, the Fc domains of the scFv-mAb format generally include skew variants (e.g. a set of amino acid substitutions as shown in FIG. 1, with particularly useful skew variants being selected from the group consisting of S364K/E357Q:L368D/K370S; L368D/K370S:S364K; L368E/K370S:S364K; T411T/E360E/Q362E:D401K; L368D/K370S:S364K/E357L, K370S:S364K/E357Q, T366S/L368A/Y407V:T366W and T366S/L368A/Y407V/Y349C:T366W/S354C), optionally ablation variants (including those shown in FIG. 3), optionally charged scFv linkers (including those shown in FIG. 5) and the heavy chain comprises pI variants (including those shown in FIG. 2).

In some embodiments, the scFv-mAb format includes skew variants, pI variants, and ablation variants. Accordingly, some embodiments include scFv-mAb formats that comprise: a) a first monomer that comprises the skew variants S364K/E357Q, the ablation variants E233P/L234V/L235A/G236del/S267K, and a variable heavy domain that, with the variable light domain of the common light chain, makes up an Fv that binds to CLDN18.2 as outlined herein, and a scFv domain that binds to CD3; b) a second monomer that comprises the skew variants L368D/K370S, the pI variants N208D/Q295E/N384D/Q418E/N421D, the ablation variants E233P/L234V/L235A/G236del/S267K, and a variable heavy domain that, with the variable light domain of the common light chain, makes up an Fv that binds to CLDN18.2 as outlined herein; and c) a common light chain comprising a variable light domain and a constant light domain.

In some embodiments, the scFv-mAb format includes skew variants, pI variants, ablation variants and FcRn variants. Accordingly, some embodiments include scFv-mAb formats that comprise: a) a first monomer that comprises the skew variants S364K/E357Q, the ablation variants E233P/L234V/L235A/G236del/S267K, the FcRn variants M428L/N434S and a variable heavy domain that, with the variable light domain of the common light chain, makes up an Fv that binds to CLDN18.2 as outlined herein, and a scFv domain that binds to CD3; b) a second monomer that comprises the skew variants L368D/K370S, the pI variants N208D/Q295E/N384D/Q418E/N421D, the ablation variants E233P/L234V/L235A/G236del/S267K, the FcRn variants M428L/N434S and a variable heavy domain that, with the variable light domain of the common light chain, makes up an Fv that binds to CLDN18.2 as outlined herein; and c) a common light chain comprising a variable light domain and a constant light domain.

For the mAb-scFv format backbone 1 (optionally including M428L/N434S) from FIG. 10, CD3 binding domain sequences finding particular use include, but are not limited to, anti-CD3 H1.30_L1.47, anti-CD3 H1.32_L1.47, anti-CD3 H1.89_L1.47, anti-CD3 H1.90_L1.47, anti-CD3 H1.33_L1.47 and anti-CD3 H1.31_L1.47, as depicted in FIG. 12.

Dual scFv Formats

The present invention also provides dual scFv formats as are known in the art and shown in FIG. 42B. In this embodiment, the CLDN18.2 x CD3 heterodimeric bispecific antibody is made up of two scFv-Fc monomers (both in either (VH-scFv linker-VL-[optional domain linker]-CH2-CH3) format or (VL-scFv linker-VH-[optional domain linker]-CH2-CH3) format, or with one monomer in one orientation and the other in the other orientation.

The present invention provides dual scFv formats where the CD3 binding domain sequences are as shown in FIG. 12 and wherein the CLDN18.2 binding domain sequences are as shown in FIG. 10.

In some embodiments, the dual scFv format includes skew variants, pI variants, and ablation variants. Accordingly, some embodiments include dual scFv formats that comprise: a) a first monomer that comprises the skew variants S364K/E357Q, the ablation variants E233P/L234V/L235A/G236del/S267K, and a first scFv that binds either CD3 or CLDN18.2; and b) a second monomer that comprises the skew variants L368D/K370S, the pI variants N208D/Q295E/N384D/ Q418E/N421D, the ablation variants E233P/L234V/L235A/G236del/S267K, and a second scFv that binds either CD3 or CLDN18.2.

In some embodiments, the dual scFv format includes skew variants, pI variants, ablation variants and FcRn variants. In some embodiments, the dual scFv format includes skew variants, pI variants, and ablation variants. Accordingly, some embodiments include dual scFv formats that comprise: a) a first monomer that comprises the skew variants S364K/E357Q, the ablation variants E233P/L234V/L235A/G236del/S267K, the FcRn variants M428L/N434S and a first scFv that binds either CD3 or CLDN18.2; and b) a second monomer that comprises the skew variants L368D/K370S, the pI variants N208D/Q295E/N384D/Q418E/N421D, the ablation variants E233P/L234V/L235A/G236del/S267K, the FcRn variants M428L/N434S and a second scFv that binds either CD3 or CLDN18.2.

For the dual scFv format, CD3 binding domain sequences finding particular use include, but are not limited to, anti-CD3 H1.30_L1.47, anti-CD3 H1.32_L1.47, anti-CD3 H1.89_L1.47, anti-CD3 H1.90_L1.47, anti-CD3 H1.33_L1.47 and anti-CD3 H1.31_L1.47, as well as depicted in FIG. 12.

### I. Antigen Binding Domains to Target Antigens

The bispecific antibodies of the invention have two different antigen binding domains (ABDs) that bind to two different target checkpoint antigens (“target pairs”), in either bivalent, bispecific formats or trivalent, bispecific formats as generally shown in FIG. 1. Note that generally these bispecific antibodies are named “anti-CLDN18.2 X anti-CD3”, or generally simplistically or for ease (and thus interchangeably) as “CLDN18.2 X CD3”, etc. for each pair. Note that unless specified herein, the order of the antigen list in the name does not confer structure; that is a CLDN18.2 X CD3 bottle opener antibody can have the scFv bind to CLDN18.2 or CD3, although in some cases, the order specifies structure as indicated.

As is more fully outlined herein, these combinations of ABDs can be in a variety of formats, as outlined below, generally in combinations where one ABD is in a Fab format and the other is in an scFv format. As discussed herein and shown in FIG. 42, some formats use a single Fab and a single scFv (FIGS. 42A, C and D), and some formats use two Fabs and a single scFv (FIGS. 42E, F, and I).

### Antigen Binding Domains

As discussed herein, the subject heterodimeric antibodies include two antigen binding domains (ABDs), each of which bind to CLDN18.2 or CD3. As outlined herein, these heterodimeric antibodies can be bispecific and bivalent (each antigen is bound by a single ABD, for example, in the format depicted in FIG. 42A), or bispecific and trivalent (one antigen is bound by a single ABD and the other is bound by two ABDs, for example as depicted in FIG. 42F).

In addition, in general, one of the ABDs comprises a scFv as outlined herein, in an orientation from N- to C-terminus of VH-scFv linker-VL or VL-scFv linker-VH. One or both of the other ABDs, according to the format, generally is a Fab, comprising a VH domain on one protein chain (generally as a component of a heavy chain) and a VL on another protein chain (generally as a component of a light chain).

The invention provides a number of ABDs that bind to a number of different checkpoint proteins, as outlined below. As will be appreciated by those in the art, any set of 6 CDRs or VH and VL domains can be in the scFv format or in the Fab format, which is then added to the heavy and light constant domains, where the heavy constant domains comprise variants (including within the CH1 domain as well as the Fc domain). The scFv sequences contained in the sequence listing utilize a particular charged linker, but as outlined herein, uncharged or other charged linkers can be used, including those depicted in FIG. 5.

In addition, as discussed above, the numbering used in the Sequence Listing for the identification of the CDRs is Kabat, however, different numbering can be used, which will change the amino acid sequences of the CDRs as shown in Table 1.

For all of the variable heavy and light domains listed herein, further variants can be made. As outlined herein, in some embodiments the set of 6 CDRs can have from 0, 1, 2, 3, 4 or 5 amino acid modifications (with amino acid substitutions finding particular use), as well as changes in the framework regions of the variable heavy and light domains, as long as the frameworks (excluding the CDRs) retain at least about 80, 85 or 90% identity to a human germline sequence selected from those listed in FIG. 1 of U.S. Pat. No.7,657,380, which Figure and Legend is incorporated by reference in its entirety herein. Thus, for example, the identical CDRs as described herein can be combined with different framework sequences from human germline sequences, as long as the framework regions retain at least 80, 85 or 90% identity to a human germline sequence selected from those listed in FIG. 1 of U.S. Pat. No. 7,657,380. Alternatively, the CDRs can have amino acid modifications (e.g. from 1, 2, 3, 4 or 5 amino acid modifications in the set of CDRs (that is, the CDRs can be modified as long as the total number of changes in the set of 6 CDRs is less than 6 amino acid modifications, with any combination of CDRs being changed; e.g. there may be one change in VLCDR1, two in VHCDR2, none in VHCDR3, etc.)), as well as having framework region changes, as long as the framework regions retain at least 80, 85 or 90% identity to a human germline sequence selected from those listed in FIG. 1 of U.S. Pat. No. 7,657,380.

### CLDN18.2 Antigen Binding Domains

In some embodiments, one of the ABDs binds CLDN18.2. Suitable sets of 6 CDRs and/or VH and VL domains are depicted in FIG. 10.

As will be appreciated by those in the art, suitable CLDN18.2 binding domains can comprise a set of 6 CDRs as depicted in the Figures, either as they are underlined or, in the case where a different numbering scheme is used as described herein and as shown in Table 1, as the CDRs that are identified using other alignments within the VH and VL sequences of those depicted in FIG. 10. Suitable ABDs can also include the entire VH and VL sequences as depicted in these sequences and Figures, used as scFvs or as Fabs. In many of the embodiments herein that contain an Fv to CLDN18.2, it is the Fab monomer that binds CLDN18.2.

In addition to the parental CDR sets disclosed in the figures and sequence listing that form an ABD to CLDN18.2, the invention provides variant CDR sets. In one embodiment, a set of 6 CDRs can have 1, 2, 3, 4 or 5 amino acid changes from the parental CDRs, as long as the CLDN18.2 ABD is still able to bind to the target antigen, as measured by at least one of a Biacore, surface plasmon resonance (SPR) and/or BLI (biolayer interferometry, e.g. Octet assay) assay, with the latter finding particular use in many embodiments.

In addition to the parental variable heavy and variable light domains disclosed herein that form an ABD to CLDN18.2, the invention provides variant VH and VL domains. In one embodiment, the variant VH and VL domains each can have from 1, 2, 3, 4, 5, 6, 7, 8, 9 or 10 amino acid changes from the parental VH and VL domain, as long as the ABD is still able to bind to the target antigen, as measured at least one of a Biacore, surface plasmon resonance (SPR) and/or BLI (biolayer interferometry, e.g. Octet assay) assay, with the latter finding particular use in many embodiments. In another embodiment, the variant VH and VL are at least 90, 95, 97, 98 or 99% identical to the respective parental VH or VL, as long as the ABD is still able to bind to the target antigen, as measured by at least one of a Biacore, surface plasmon resonance (SPR) and/or BLI (biolayer interferometry, e.g. Octet assay) assay, with the latter finding particular use in many embodiments.

Specific preferred embodiments include the H1L1 and H2L1 CLDN18.2 antigen binding domain, as a “Fab”, included within any of the bottle opener format backbones of FIG. 6.

Specific preferred embodiments include the H1L1 and H2L1 CLDN18.2 antigen binding domain, as a “Fab”, included within any of the 2+1 format backbones of FIG. 41.

### CD3 Antigen Binding Domains

In some embodiments, one of the ABDs binds CD3. Suitable sets of 6 CDRs and/or VH and VL domains, as well as scFv sequences, are depicted in FIGS. 12 and 13 and the Sequence Listing. CD3 binding domain sequences that are of particular use include, but are not limited to, anti-CD3 H1.30_L1.47, anti-CD3 H1.32_L1.47, anti-CD3 H1.89_L1.47, anti-CD3 H1.90_L1.47, anti-CD3 H1.33_L1.47 and anti-CD3 H1.31_L1.47, as depicted in FIG. 12.

As will be appreciated by those in the art, suitable CD3 binding domains can comprise a set of 6 CDRs as depicted in FIG. 12, either as they are underlined or, in the case where a different numbering scheme is used as described herein and as shown in Table 1, as the CDRs that are identified using other alignments within the VH and VL sequences of those depicted in FIG. 12. Suitable ABDs can also include the entire VH and VL sequences as depicted in these sequences and Figures, used as scFvs or as Fabs. In many of the embodiments herein that contain an Fv to CD3, it is the scFv monomer that binds CD3.

In addition to the parental CDR sets disclosed in the figures and sequence listing that form an ABD to CD3, the invention provides variant CDR sets. In one embodiment, a set of 6 CDRs can have 1, 2, 3, 4 or 5 amino acid changes from the parental CDRs, as long as the CD3 ABD is still able to bind to the target antigen, as measured by at least one of a Biacore, surface plasmon resonance (SPR) and/or BLI (biolayer interferometry, e.g. Octet assay) assay, with the latter finding particular use in many embodiments.

In addition to the parental variable heavy and variable light domains disclosed herein that form an ABD to CD3, the invention provides variant VH and VL domains. In one embodiment, the variant VH and VL domains each can have from 1, 2, 3, 4, 5, 6, 7, 8, 9 or 10 amino acid changes from the parental VH and VL domain, as long as the ABD is still able to bind to the target antigen, as measured at least one of a Biacore, surface plasmon resonance (SPR) and/or BLI (biolayer interferometry, e.g. Octet assay) assay, with the latter finding particular use in many embodiments. In another embodiment, the variant VH and VL are at least 90, 95, 97, 98 or 99% identical to the respective parental VH or VL, as long as the ABD is still able to bind to the target antigen, as measured by at least one of a Biacore, surface plasmon resonance (SPR) and/or BLI (biolayer interferometry, e.g. Octet assay) assay, with the latter finding particular use in many embodiments.

### J. Useful Embodiments

In one embodiment, a particular combination of skew and pI variants that finds use in the present invention is T366S/L368A/Y407V:T366W (optionally including a bridging disulfide, T366S/L368A/Y407V/Y349C:T366W/S354C) with one monomer comprises Q295E/N384D/Q418E/N481D and the other a positively charged scFv linker (when the format includes an scFv domain). As will be appreciated in the art, the “knobs in holes” variants do not change pI, and thus can be used on either monomer.

### K. Nucleic Acids of the Invention

The invention further provides nucleic acid compositions encoding the anti-CLDN18.2 antibodies provided herein, including, but not limited to, anti-CLDN18.2 x anti-CD3 bispecific antibodies and CLDN18.2 monospecific antibodies.

As will be appreciated by those in the art, the nucleic acid compositions will depend on the format and scaffold of the heterodimeric protein. Thus, for example, when the format requires three amino acid sequences, such as for the 1+1 Fab-scFv-Fc format (e.g. a first amino acid monomer comprising an Fc domain and a scFv, a second amino acid monomer comprising a heavy chain and a light chain), three nucleic acid sequences can be incorporated into one or more expression vectors for expression. Similarly, some formats (e.g. dual scFv formats such as disclosed in FIG. 1) only two nucleic acids are needed; again, they can be put into one or two expression vectors.

As is known in the art, the nucleic acids encoding the components of the invention can be incorporated into expression vectors as is known in the art, and depending on the host cells used to produce the heterodimeric antibodies of the invention. Generally the nucleic acids are operably linked to any number of regulatory elements (promoters, origin of replication, selectable markers, ribosomal binding sites, inducers, etc.). The expression vectors can be extra-chromosomal or integrating vectors.

The nucleic acids and/or expression vectors of the invention are then transformed into any number of different types of host cells as is well known in the art, including mammalian, bacterial, yeast, insect and/or fungal cells, with mammalian cells (e.g. CHO cells), finding use in many embodiments.

In some embodiments, nucleic acids encoding each monomer and the optional nucleic acid encoding a light chain, as applicable depending on the format, are each contained within a single expression vector, generally under different or the same promoter controls. In embodiments of particular use in the present invention, each of these two or three nucleic acids are contained on a different expression vector. As shown herein and in 62/025,931, hereby incorporated by reference, different vector ratios can be used to drive heterodimer formation. That is, surprisingly, while the proteins comprise first monomer:second monomer:light chains (in the case of many of the embodiments herein that have three polypeptides comprising the heterodimeric antibody) in a 1:1:2 ratio, these are not the ratios that give the best results.

The heterodimeric antibodies of the invention are made by culturing host cells comprising the expression vector(s) as is well known in the art. Once produced, traditional antibody purification steps are done, including an ion exchange chromatography step. As discussed herein, having the pIs of the two monomers differ by at least 0.5 can allow separation by ion exchange chromatography or isoelectric focusing, or other methods sensitive to isoelectric point. That is, the inclusion of pI substitutions that alter the isoelectric point (pI) of each monomer so that such that each monomer has a different pI and the heterodimer also has a distinct pI, thus facilitating isoelectric purification of the “1+1 Fab-scFv-Fc ” and “2+1” heterodimers (e.g., anionic exchange columns, cationic exchange columns). These substitutions also aid in the determination and monitoring of any contaminating dual scFv-Fc and mAb homodimers post-purification (e.g., IEF gels, cIEF, and analytical IEX columns).

### L. Biological and Biochemical Functionality of the Heterodimeric Bispecific Antibodies

Generally the bispecific CLDN18.2 x CD3 antibodies of the invention are administered to patients with cancer, and efficacy is assessed, in a number of ways as described herein. Thus, while standard assays of efficacy can be run, such as cancer load, size of tumor, evaluation of presence or extent of metastasis, etc., immuno-oncology treatments can be assessed on the basis of immune status evaluations as well. This can be done in a number of ways, including both in vitro and in vivo assays.

### M. Treatments

Once made, the compositions of the invention find use in a number of applications. CLDN18.2 is highly expressed in gastric tumors. Accordingly, the heterodimeric compositions of the invention find use in the treatment of such CLDN18.2 positive cancers.

### Antibody Compositions for In Vivo Administration

Formulations of the antibodies used in accordance with the present invention are prepared for storage by mixing an antibody having the desired degree of purity with optional pharmaceutically acceptable carriers, excipients or stabilizers (Remington's Pharmaceutical Sciences 16th edition, Osol, A. Ed. [1980]), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

### Administrative Modalities

The antibodies and chemotherapeutic agents of the invention are administered to a subject, in accord with known methods, such as intravenous administration as a bolus or by continuous infusion over a period of time.

### Treatment Modalities

In the methods of the invention, therapy is used to provide a positive therapeutic response with respect to a disease or condition. By “positive therapeutic response” is intended an improvement in the disease or condition, and/or an improvement in the symptoms associated with the disease or condition. For example, a positive therapeutic response would refer to one or more of the following improvements in the disease: (1) a reduction in the number of neoplastic cells; (2) an increase in neoplastic cell death; (3) inhibition of neoplastic cell survival; (5) inhibition (i.e., slowing to some extent, preferably halting) of tumor growth; (6) an increased patient survival rate; and (7) some relief from one or more symptoms associated with the disease or condition.

Positive therapeutic responses in any given disease or condition can be determined by standardized response criteria specific to that disease or condition. Tumor response can be assessed for changes in tumor morphology (i.e., overall tumor burden, tumor size, and the like) using screening techniques such as magnetic resonance imaging (MRI) scan, x-radiographic imaging, computed tomographic (CT) scan, bone scan imaging, endoscopy, and tumor biopsy sampling including bone marrow aspiration (BMA) and counting of tumor cells in the circulation.

In addition to these positive therapeutic responses, the subject undergoing therapy may experience the beneficial effect of an improvement in the symptoms associated with the disease.

Treatment according to the present invention includes a “therapeutically effective amount” of the medicaments used. A “therapeutically effective amount” refers to an amount effective, at dosages and for periods of time necessary, to achieve a desired therapeutic result.

A therapeutically effective amount may vary according to factors such as the disease state, age, sex, and weight of the individual, and the ability of the medicaments to elicit a desired response in the individual. A therapeutically effective amount is also one in which any toxic or detrimental effects of the antibody or antibody portion are outweighed by the therapeutically beneficial effects.

A “therapeutically effective amount” for tumor therapy may also be measured by its ability to stabilize the progression of disease. The ability of a compound to inhibit cancer may be evaluated in an animal model system predictive of efficacy in human tumors.

Alternatively, this property of a composition may be evaluated by examining the ability of the compound to inhibit cell growth or to induce apoptosis by in vitro assays known to the skilled practitioner. A therapeutically effective amount of a therapeutic compound may decrease tumor size, or otherwise ameliorate symptoms in a subject. One of ordinary skill in the art would be able to determine such amounts based on such factors as the subject's size, the severity of the subject's symptoms, and the particular composition or route of administration selected.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

The specification for the dosage unit forms of the present invention are dictated by and directly dependent on (a) the unique characteristics of the active compound and the particular therapeutic effect to be achieved, and (b) the limitations inherent in the art of compounding such an active compound for the treatment of sensitivity in individuals.

The efficient dosages and the dosage regimens for the bispecific antibodies used in the present invention depend on the disease or condition to be treated and may be determined by the persons skilled in the art.

An exemplary, non-limiting range for a therapeutically effective amount of an bispecific antibody used in the present invention is about 0.1-100 mg/kg.

All cited references are herein expressly incorporated by reference in their entirety.

Whereas particular embodiments of the invention have been described above for purposes of illustration, it will be appreciated by those skilled in the art that numerous variations of the details may be made without departing from the invention as described in the appended claims.

### 2. EXAMPLES

Examples are provided below to illustrate the present invention. These examples are not meant to constrain the present invention to any particular application or theory of operation. For all constant region positions discussed in the present invention, numbering is according to the EU index as in Kabat (Kabat et al., 1991, Sequences of Proteins of Immunological Interest, 5th Ed., United States Public Health Service, National Institutes of Health, Bethesda, entirely incorporated by reference). Those skilled in the art of antibodies will appreciate that this convention consists of nonsequential numbering in specific regions of an immunoglobulin sequence, enabling a normalized reference to conserved positions in immunoglobulin families. Accordingly, the positions of any given immunoglobulin as defined by the EU index will not necessarily correspond to its sequential sequence.

General and specific scientific techniques are outlined in US Publications 2015/0307629, 2014/0288275 and WO2014/145806, all of which are expressly incorporated by reference in their entirety and particularly for the techniques outlined therein.

### 2.1 Example 1: Prototype Anti-CLDN18.2 x anti-CD3 Bispecific Antibodies

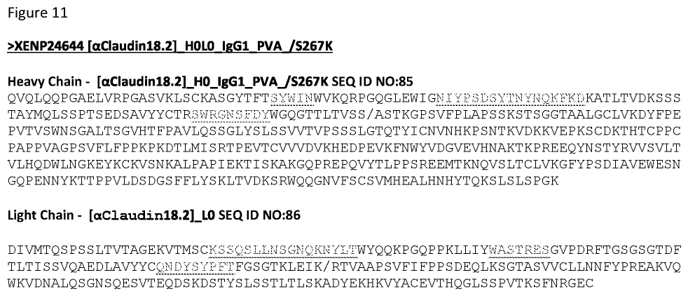

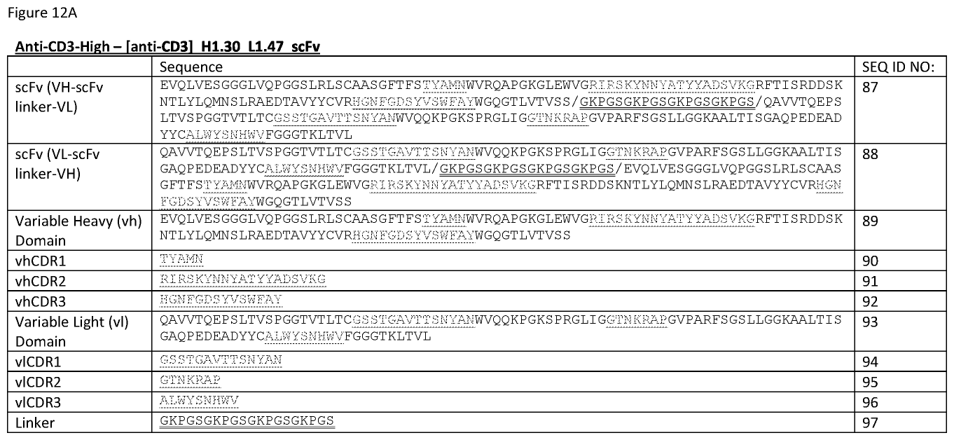

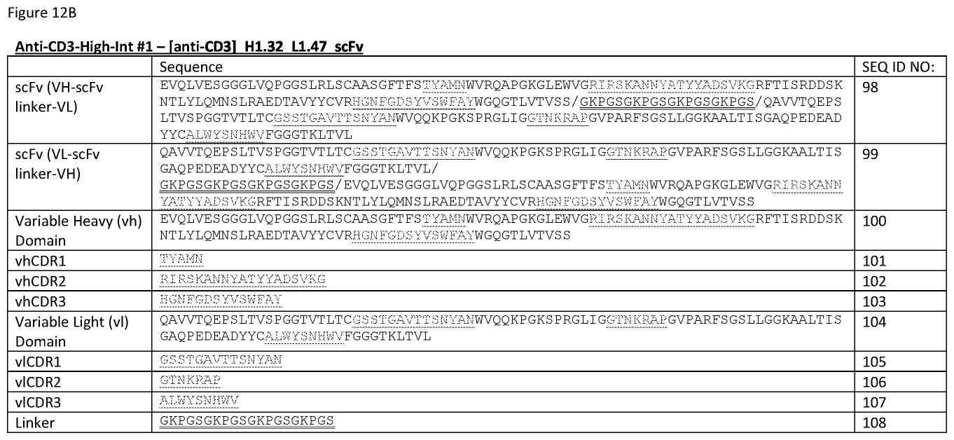

We investigated the potential of using anti-CLDN18.2 x anti-CD3 bispecific antibodies (bsAbs) to redirect CD3+ effector T cells to destroy CLDN18.2-expressing cell lines. Towards this, prototype anti-CLDN18.2 x anti-CD3 bsAbs were generated using the antigen binding domain (ABD) from a murine anti-CLDN18.2 antibody (see, e.g., U.S. Pat. No. 9,751,934, issued Sep. 5, 2017), sequences for which are depicted in FIG. 11. Sequences for anti-CD3 scFvs having different affinities for CD3 which use in the bsAbs are depicted in FIG. 12.

(a) 1A: Production of Prototype Anti-CLDN18.2 x anti-CD3 bsAbs

Schematics for illustrative anti-CLDN18.2 x anti-CD3 bsAbs are depicted in FIG. 13. FIG. 13A depicts the “1+1 Fab-scFv-Fc” format which comprises a single-chain Fv (“scFv”) recombinantly fused to one side of a heterodimeric Fc, a heavy chain variable region (VH) recombinantly fused to the other side of the heterodimeric Fc, and a light chain (LC) transfected separately so that a Fab domain is formed with the variable heavy domain. FIG. 13B depicts the “2+1 Fab₂-scFv-Fc” format which comprises a VH domain recombinantly fused to an scFv fused to one side of a heterodimeric Fc, a VH domain recombinantly fused to the other side of the heterodimeric Fc, and a LC transfected separately so that Fab domains are formed with the VH domain.

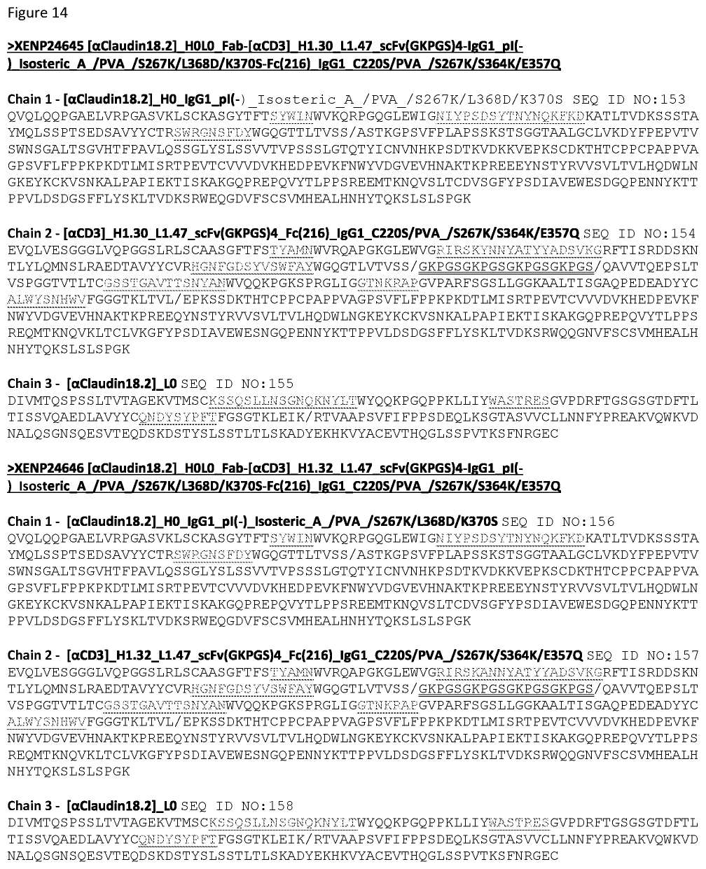

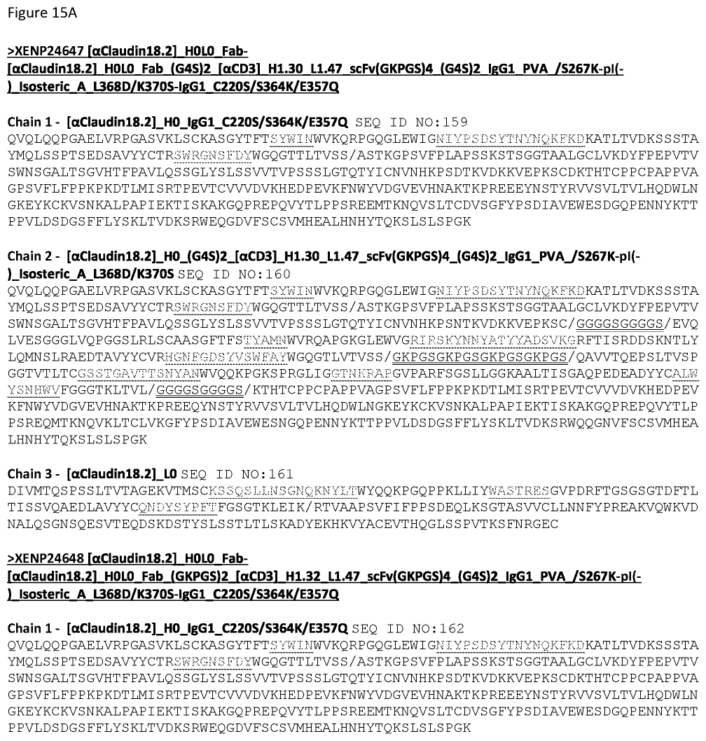

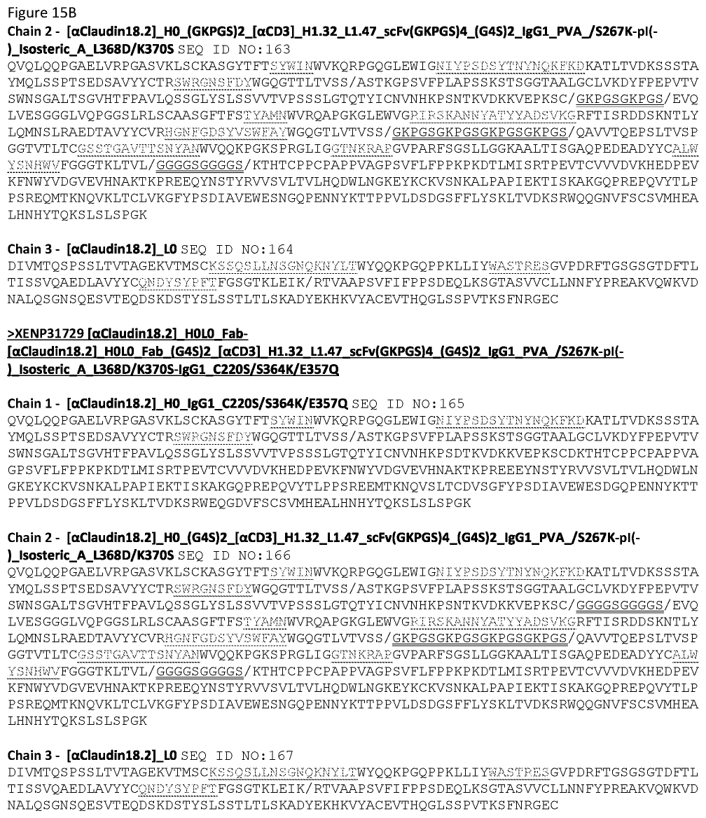

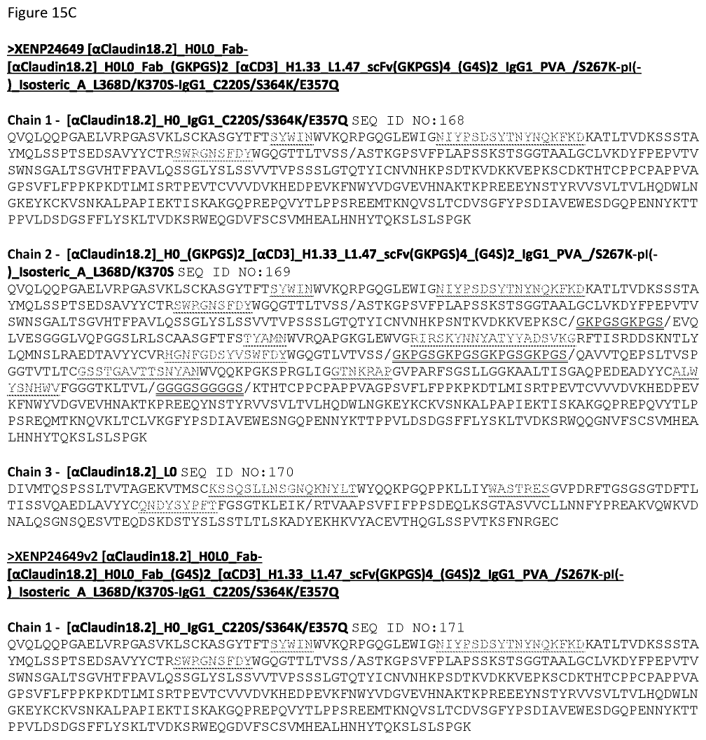

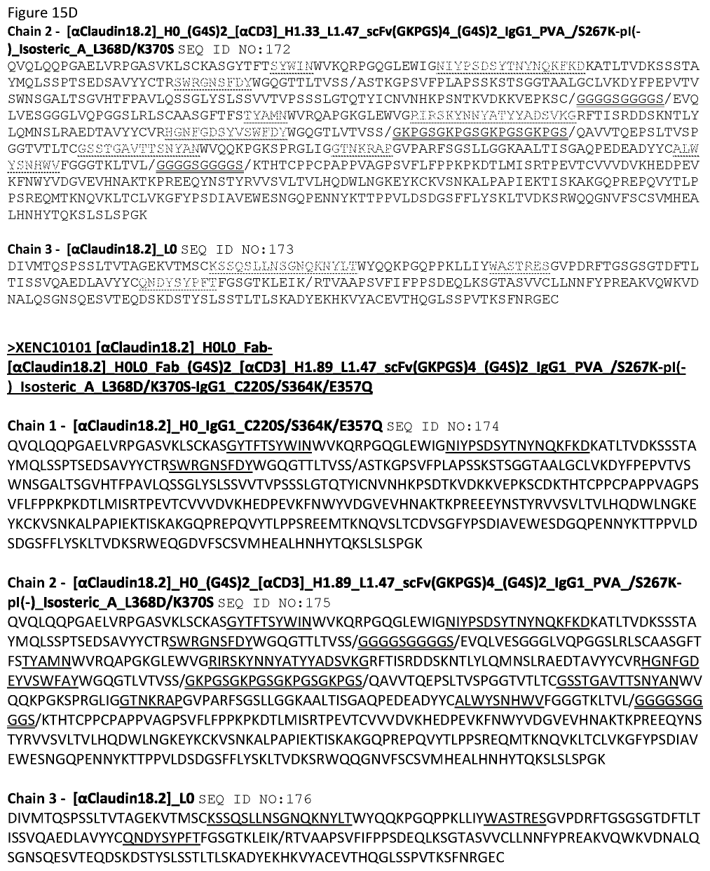

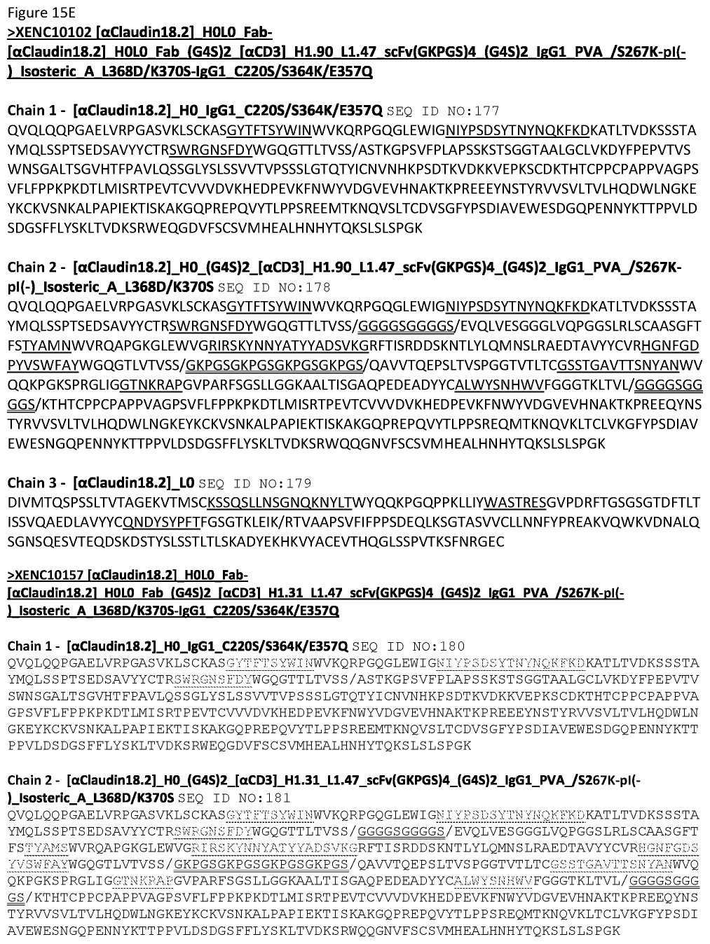

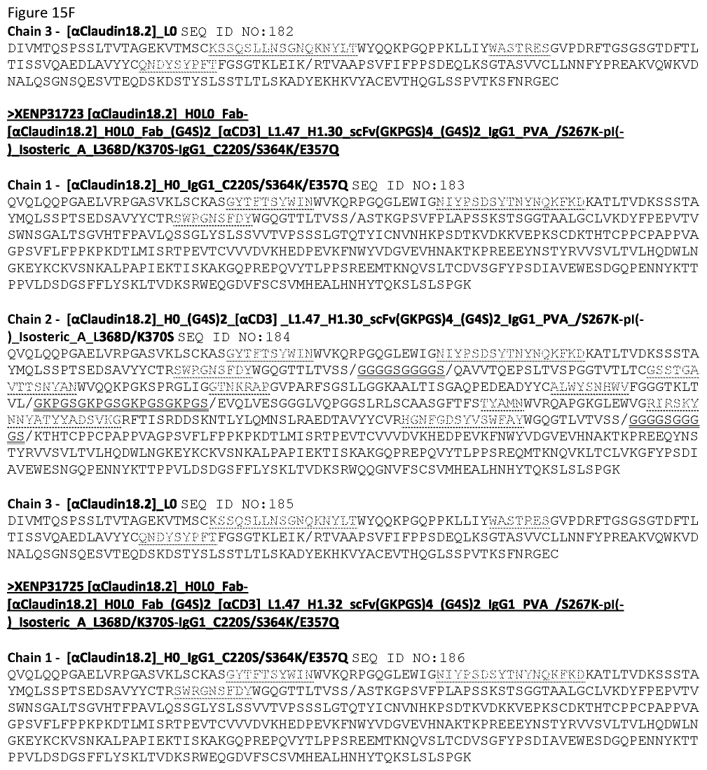

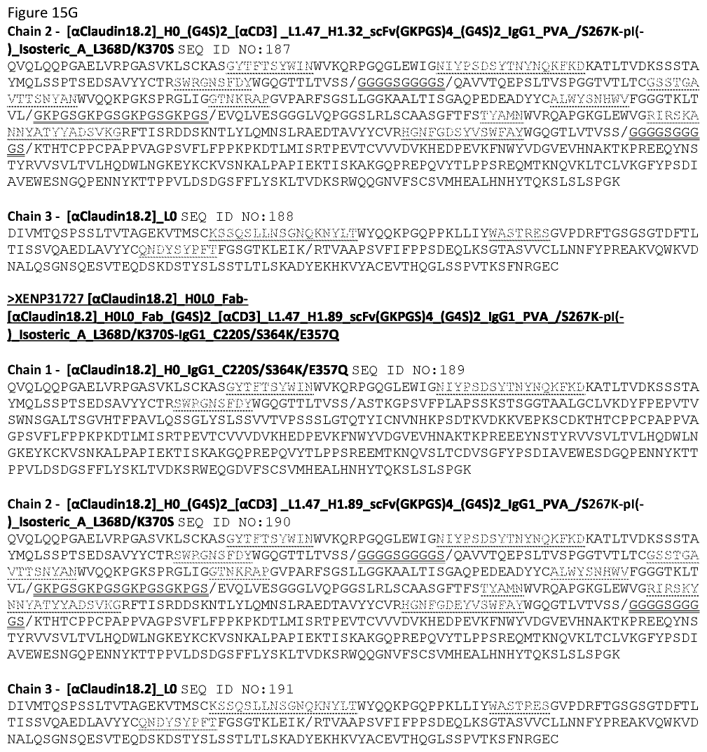

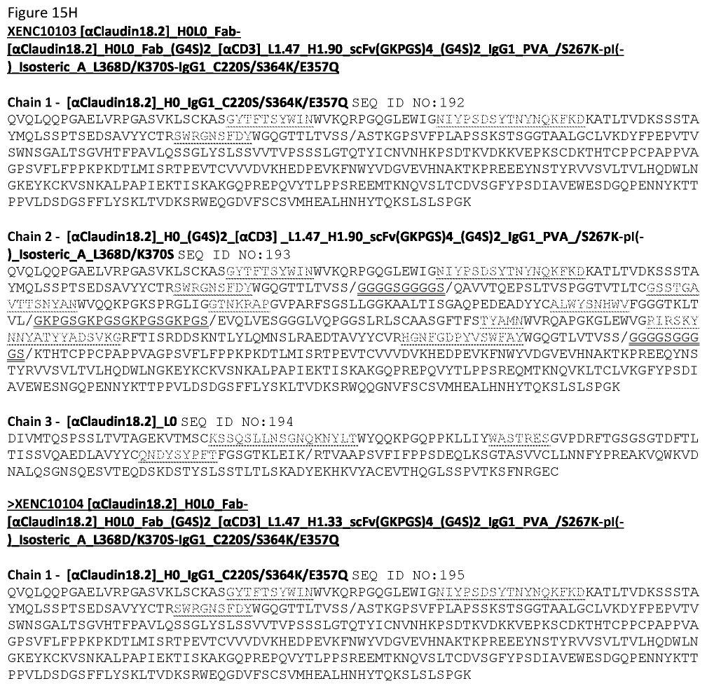

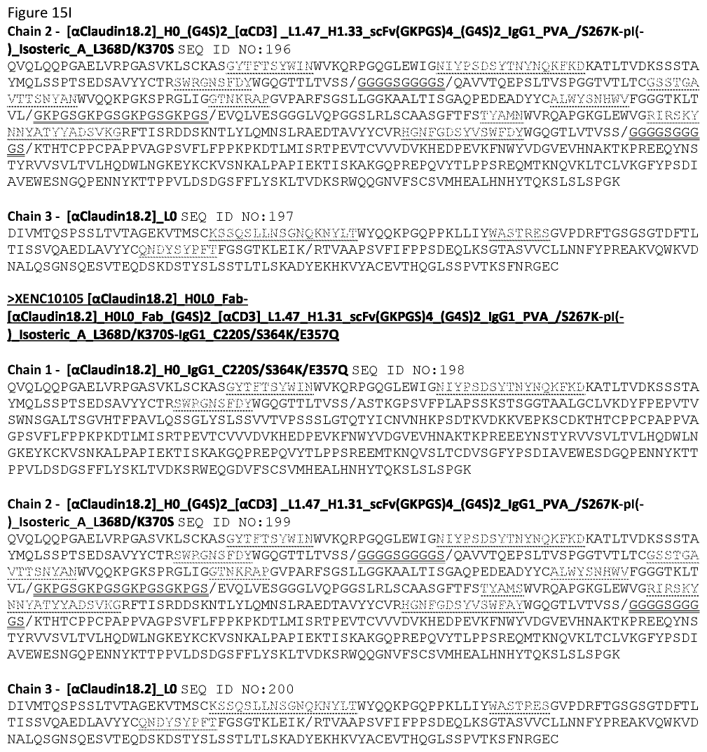

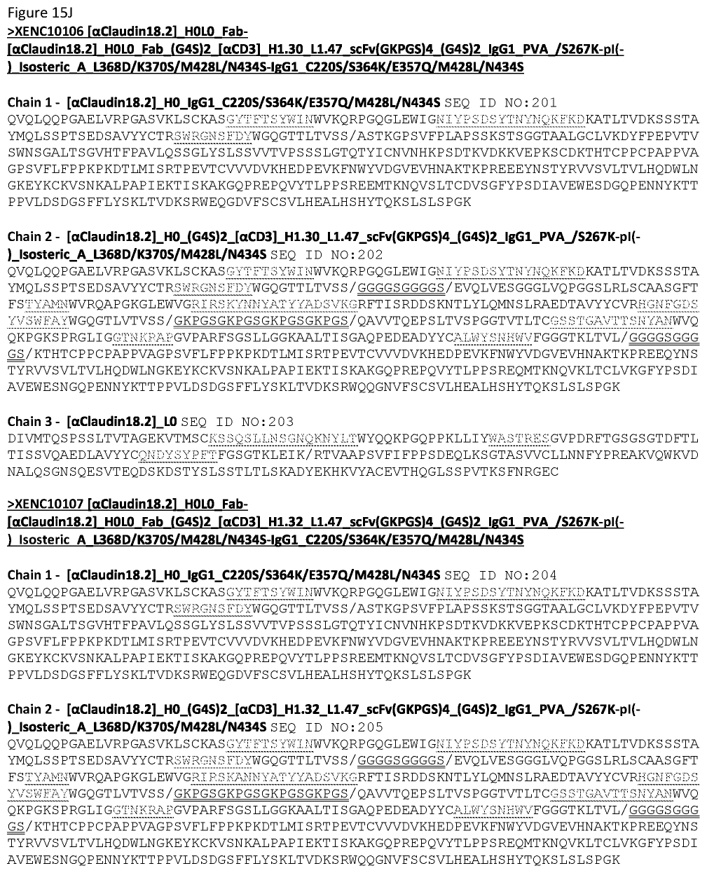

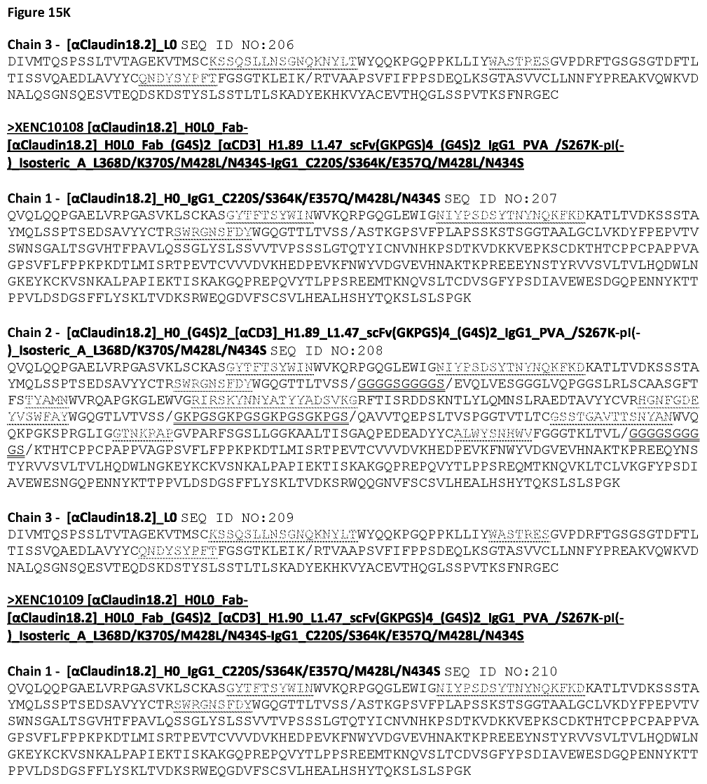

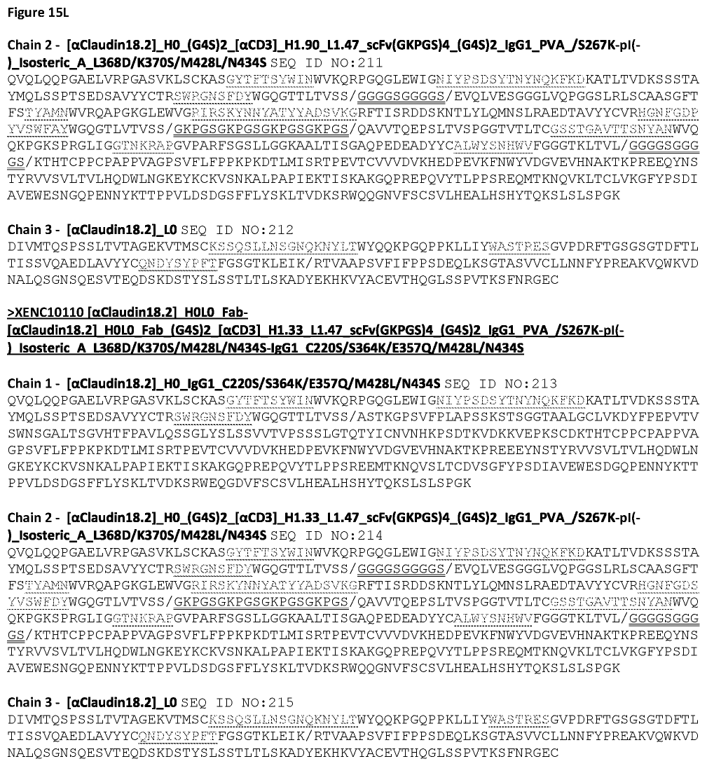

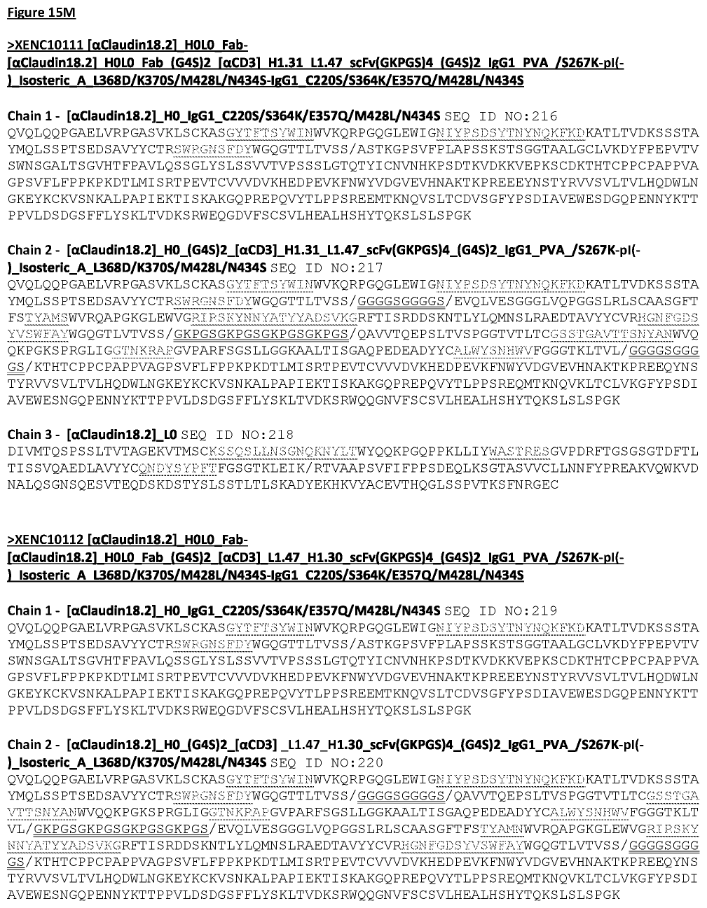

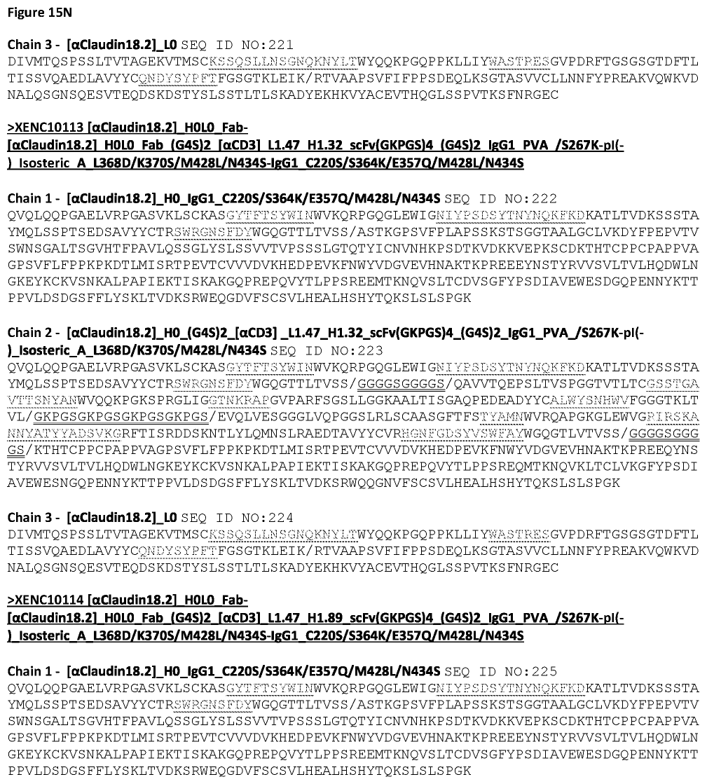

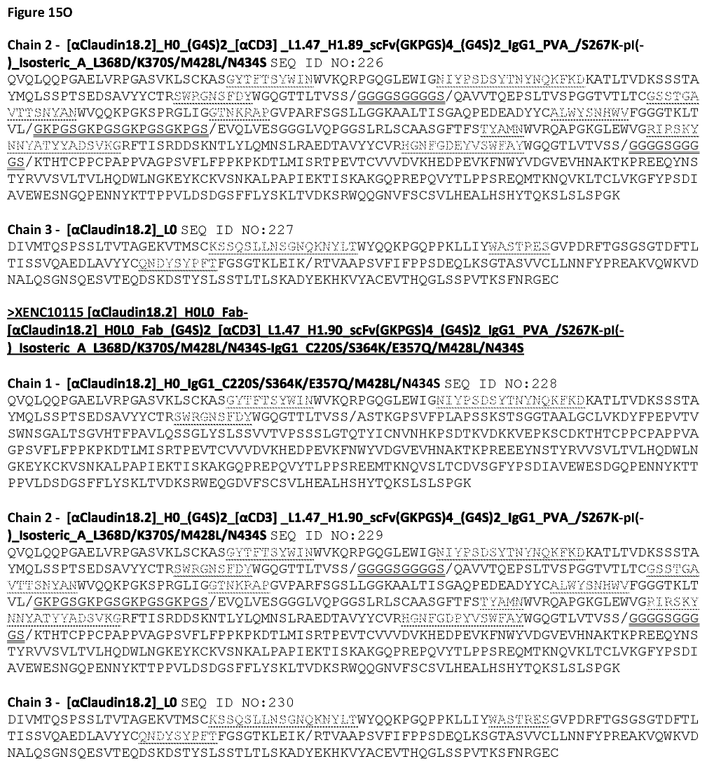

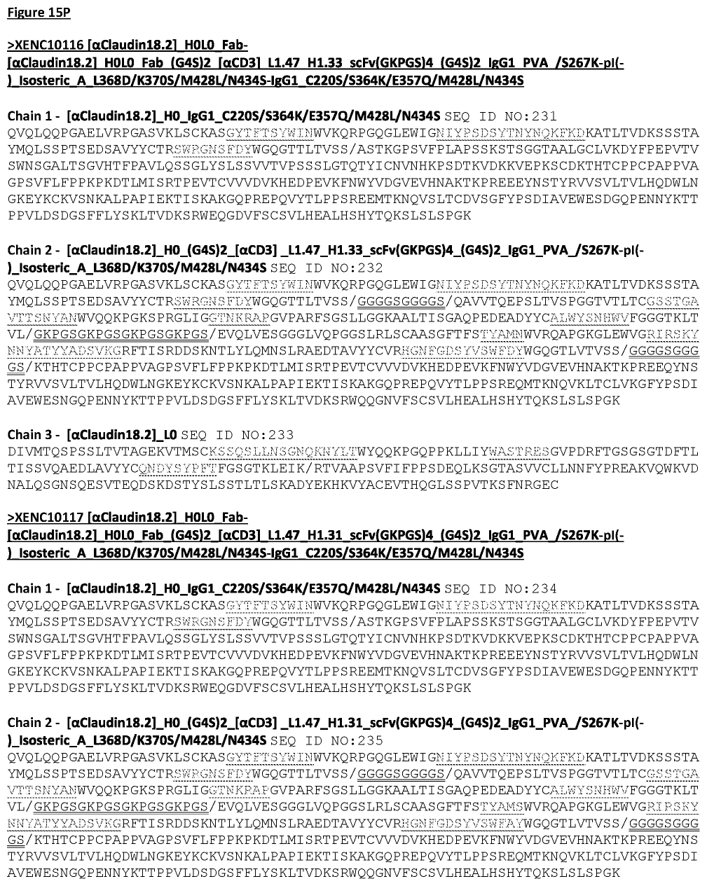

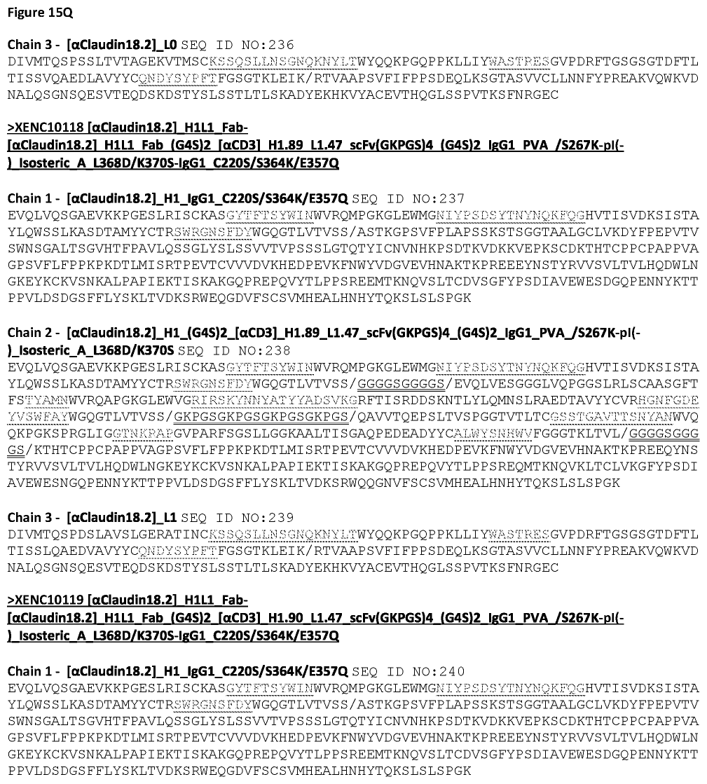

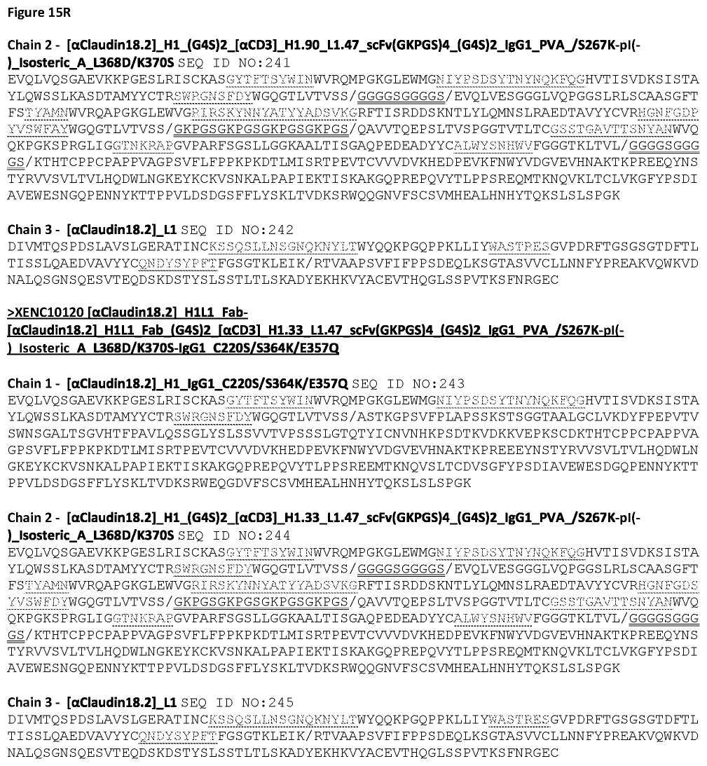

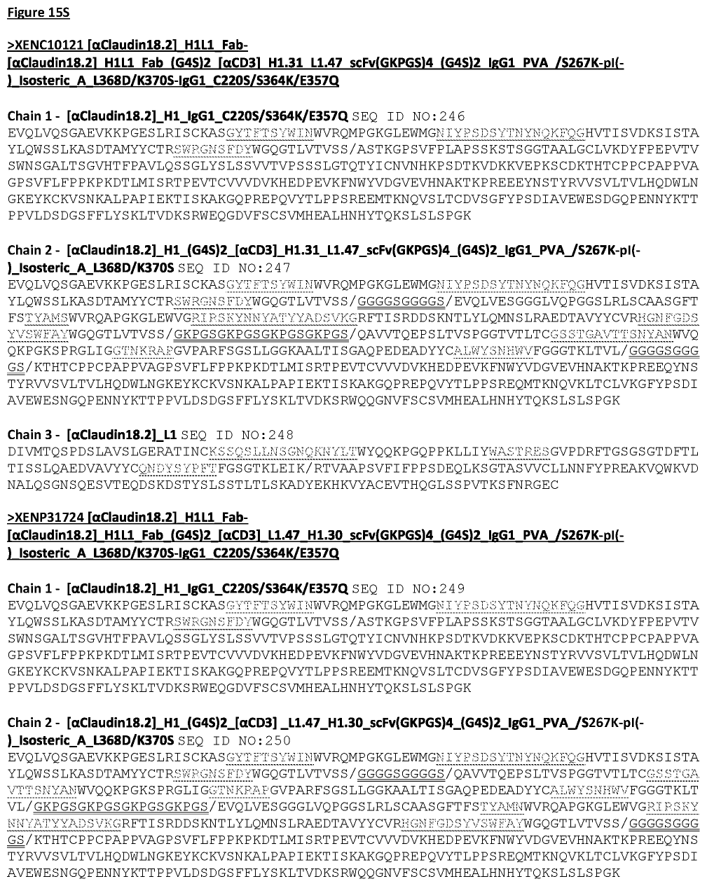

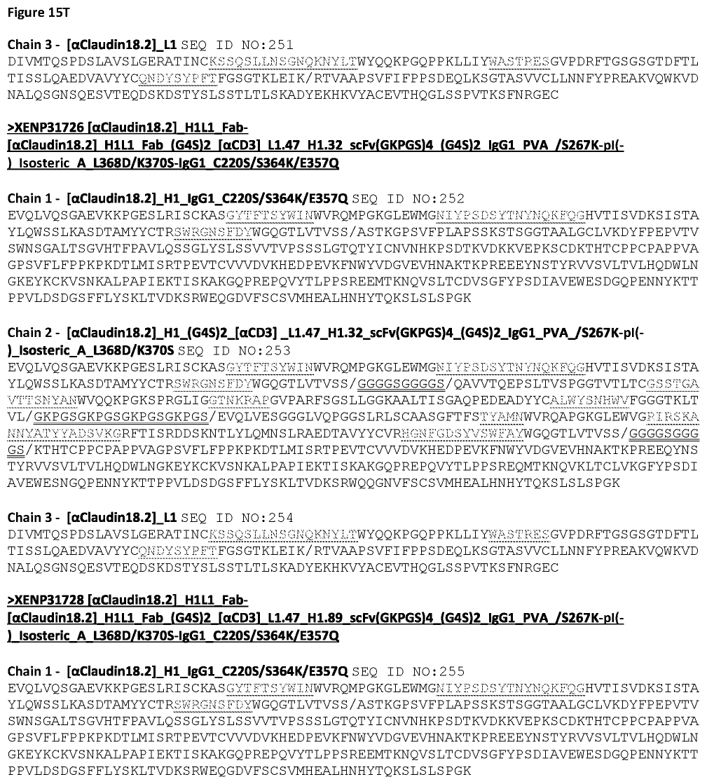

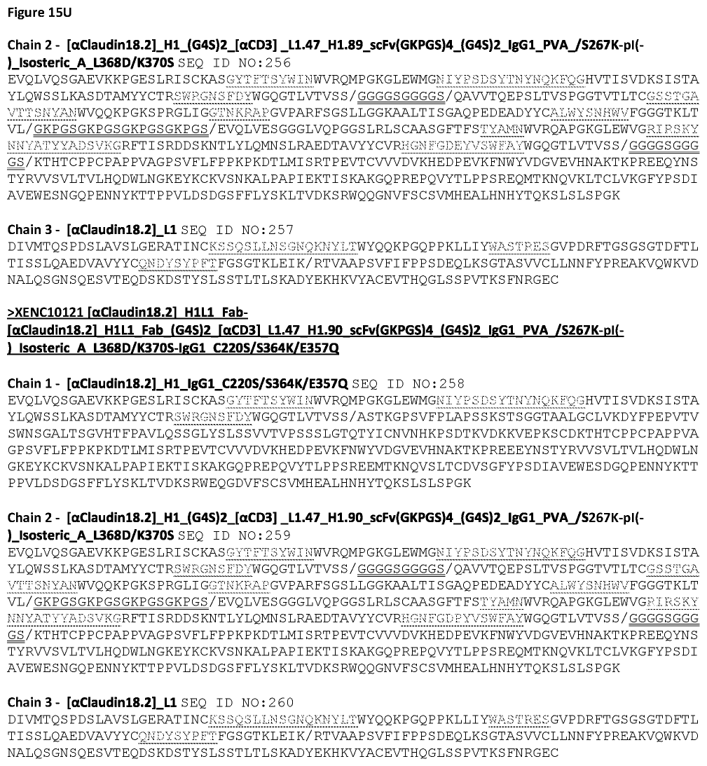

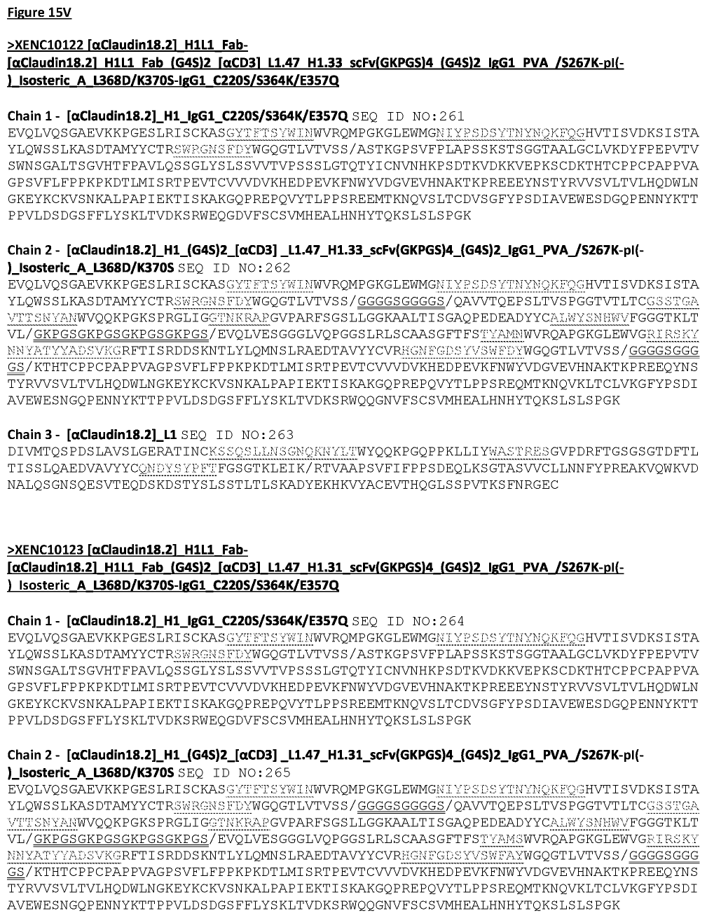

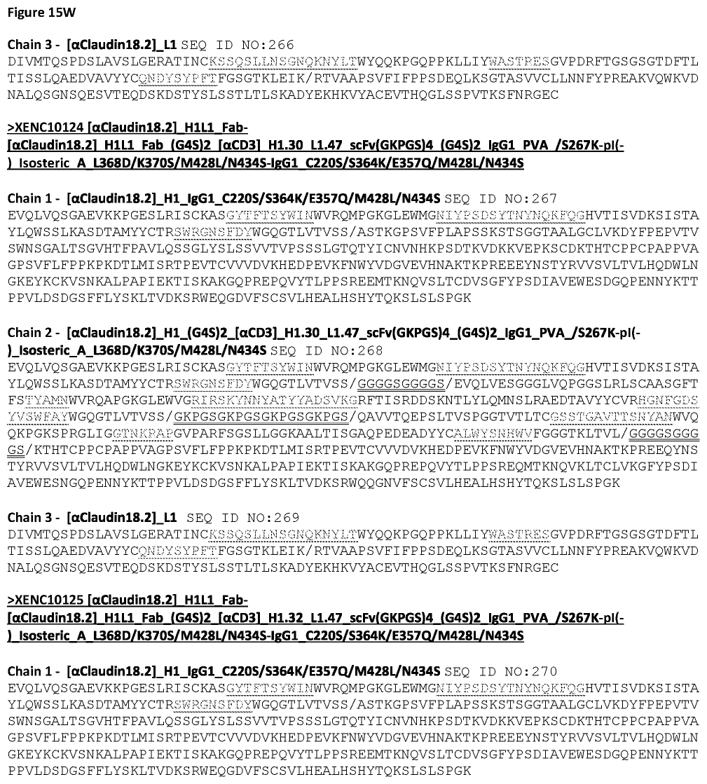

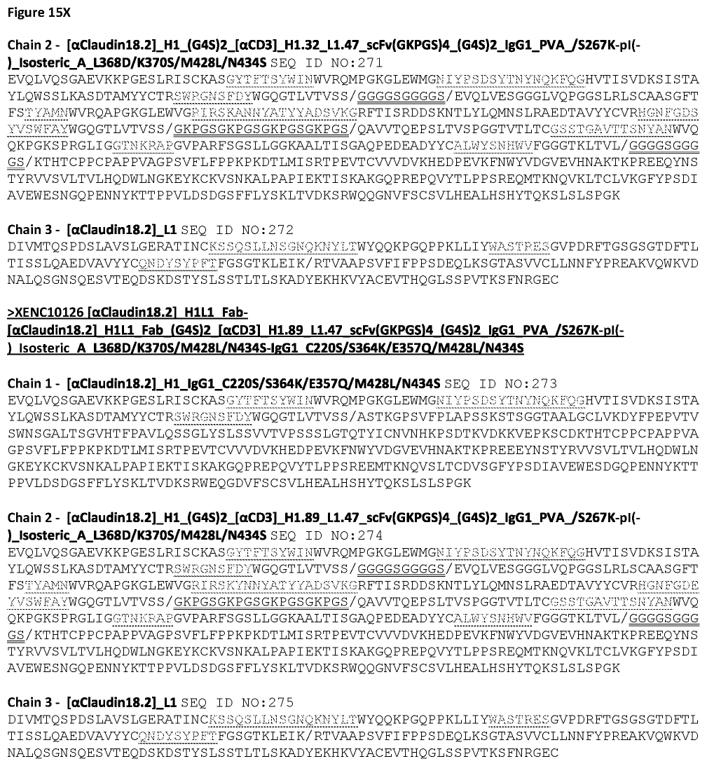

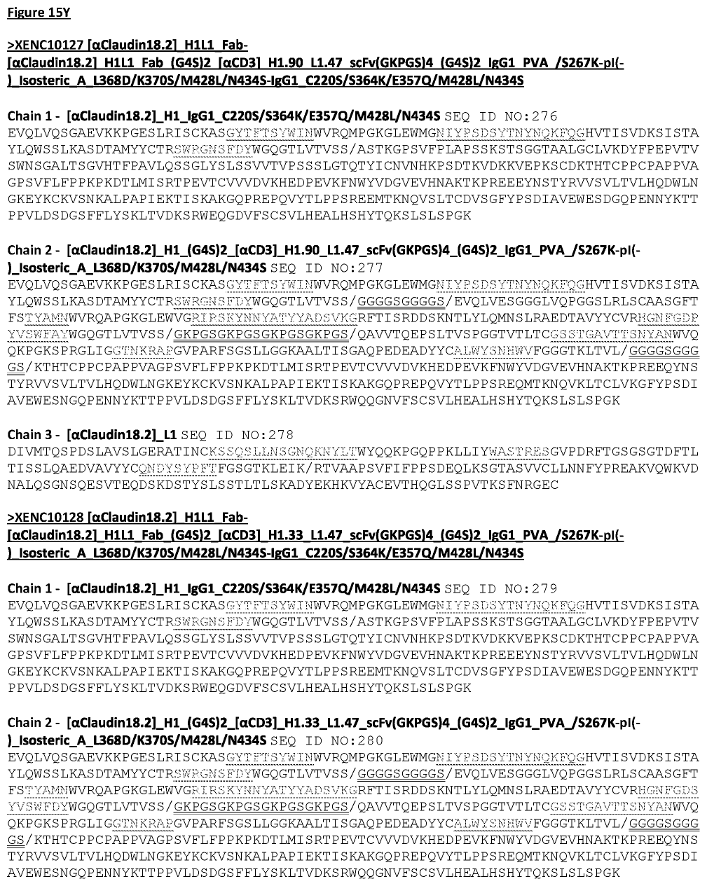

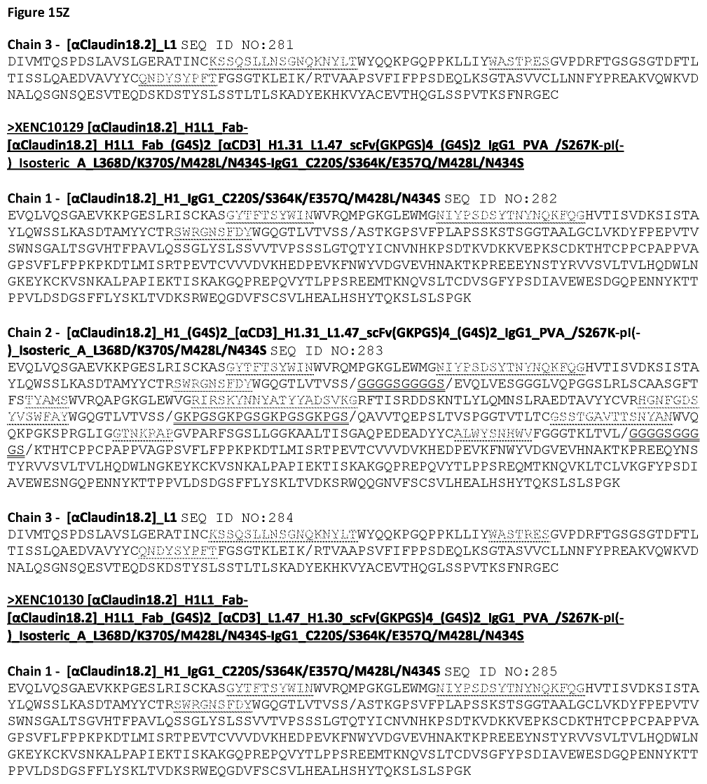

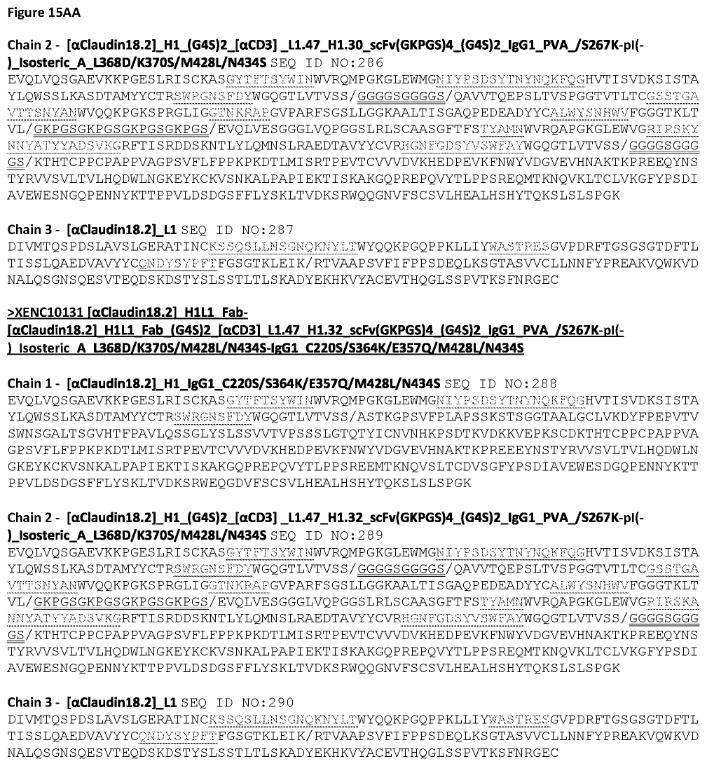

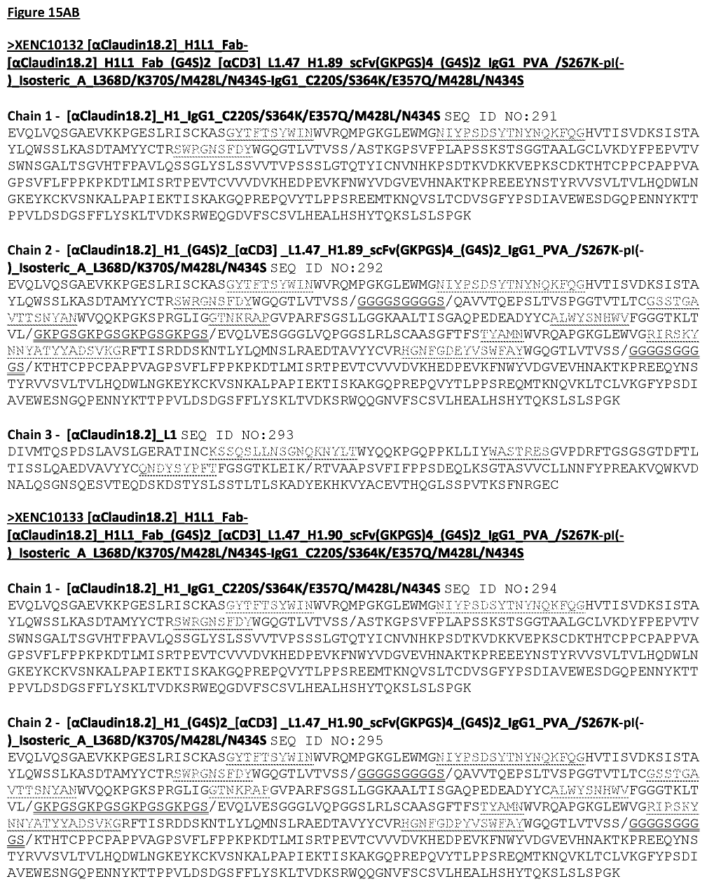

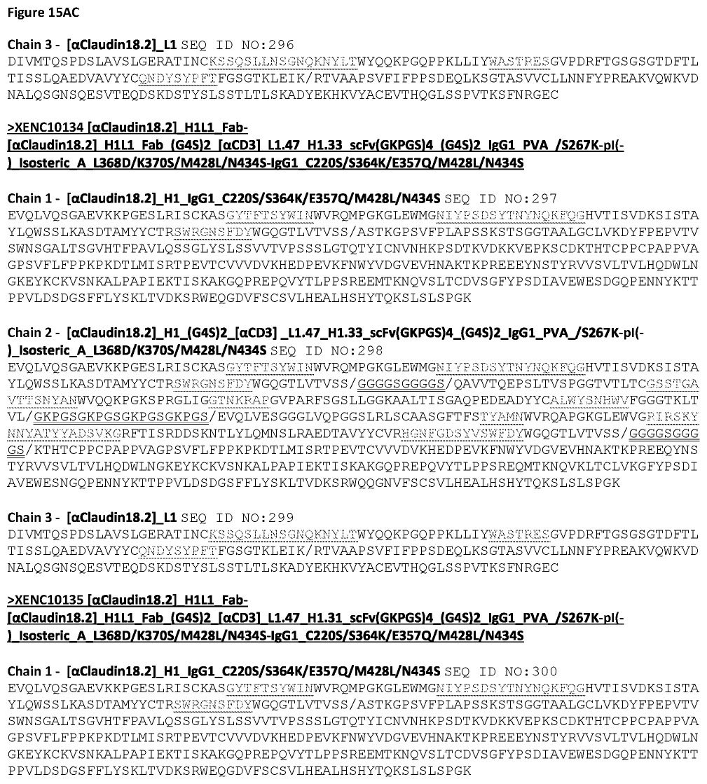

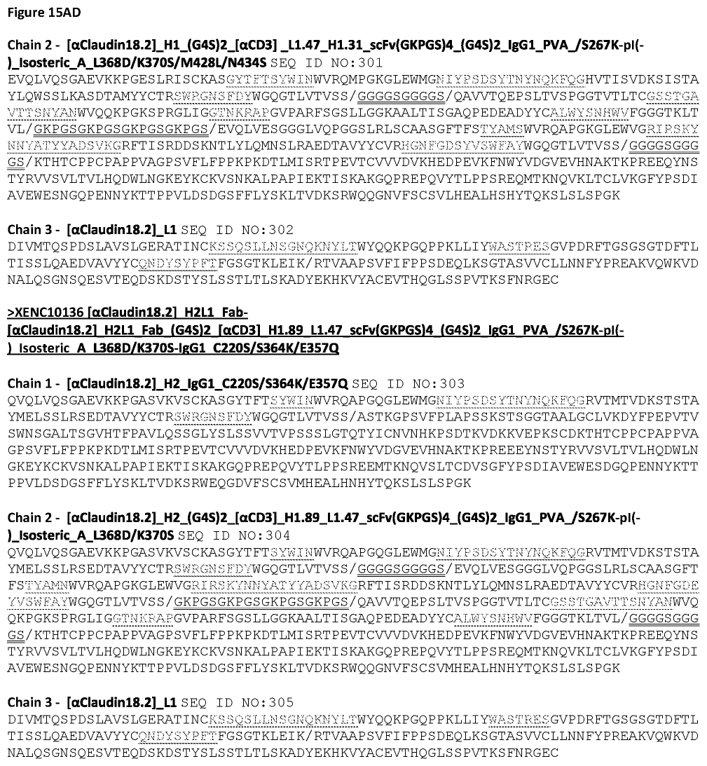

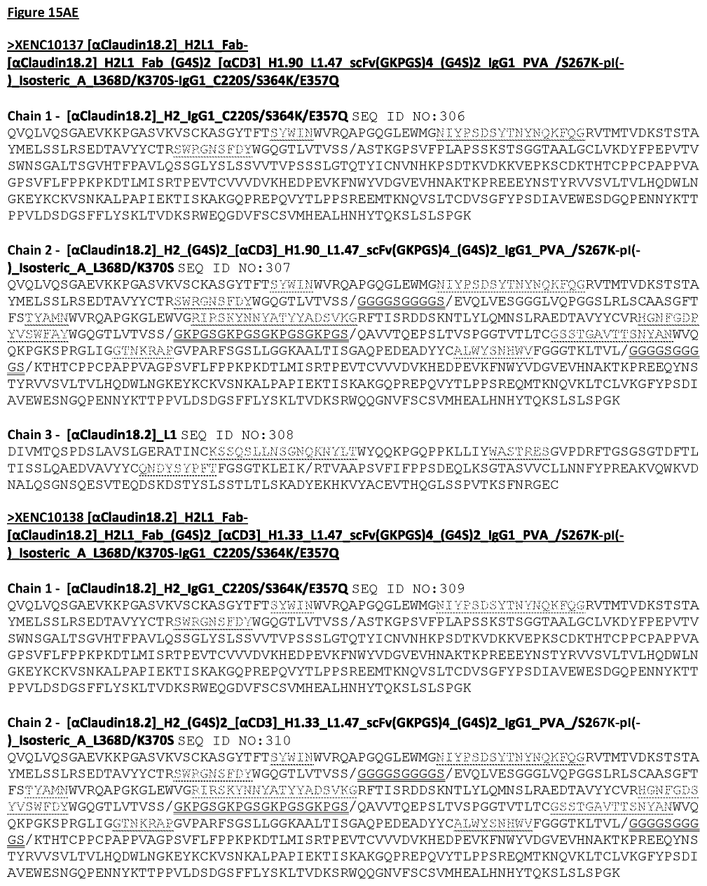

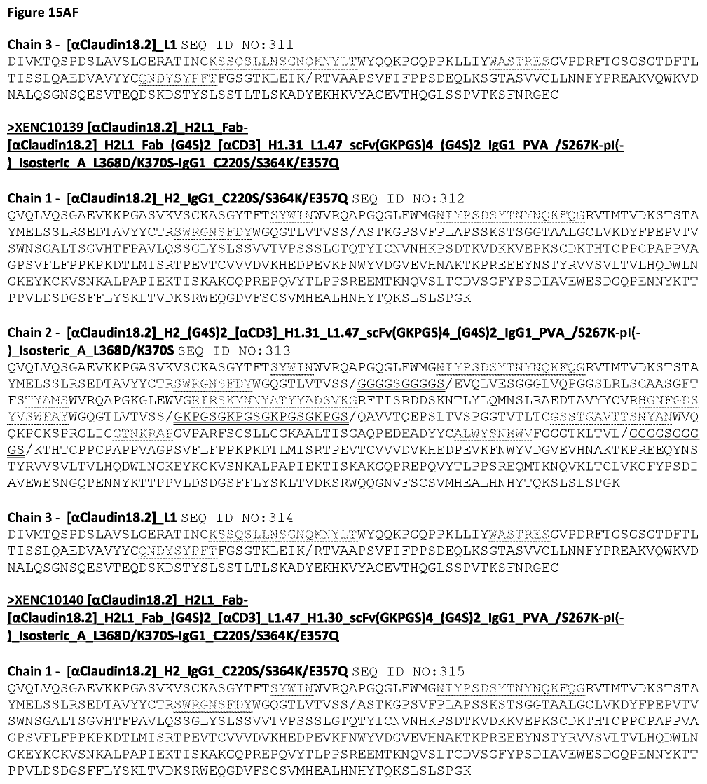

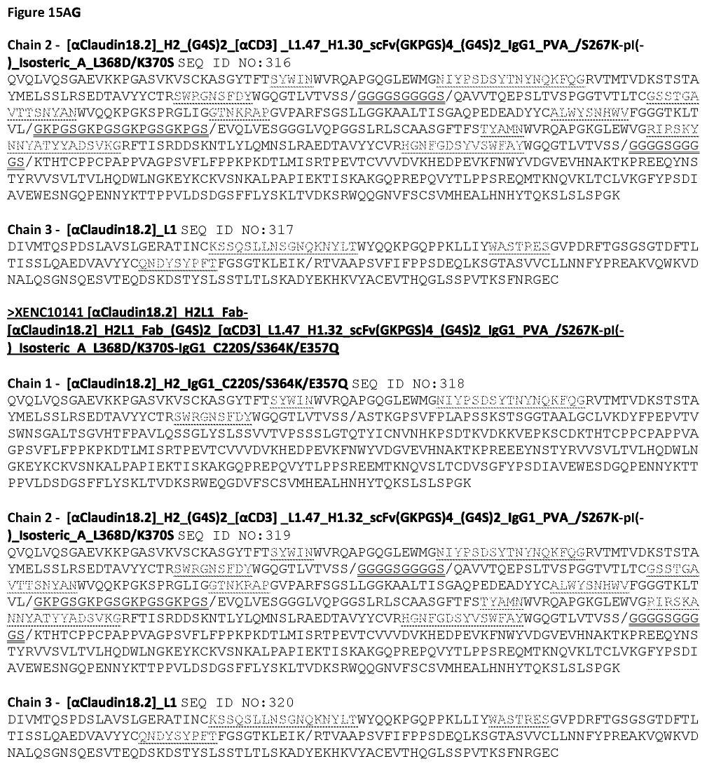

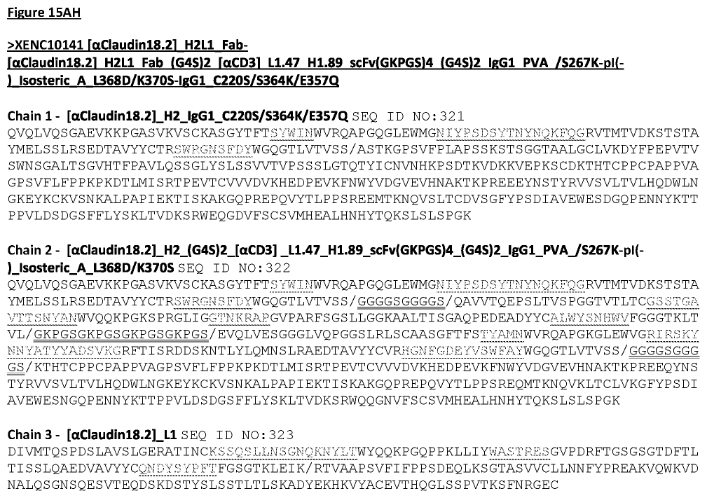

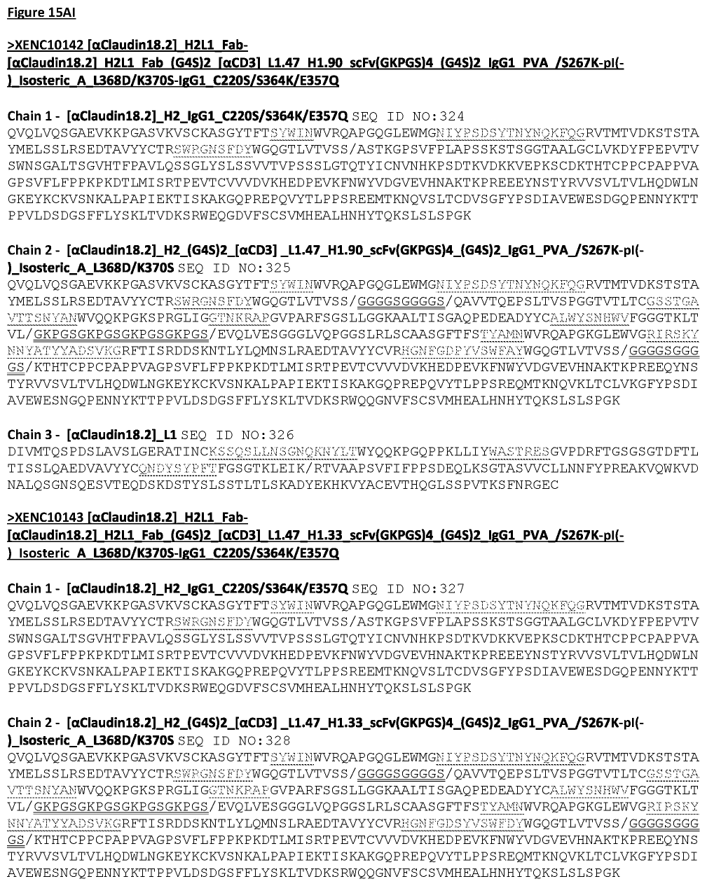

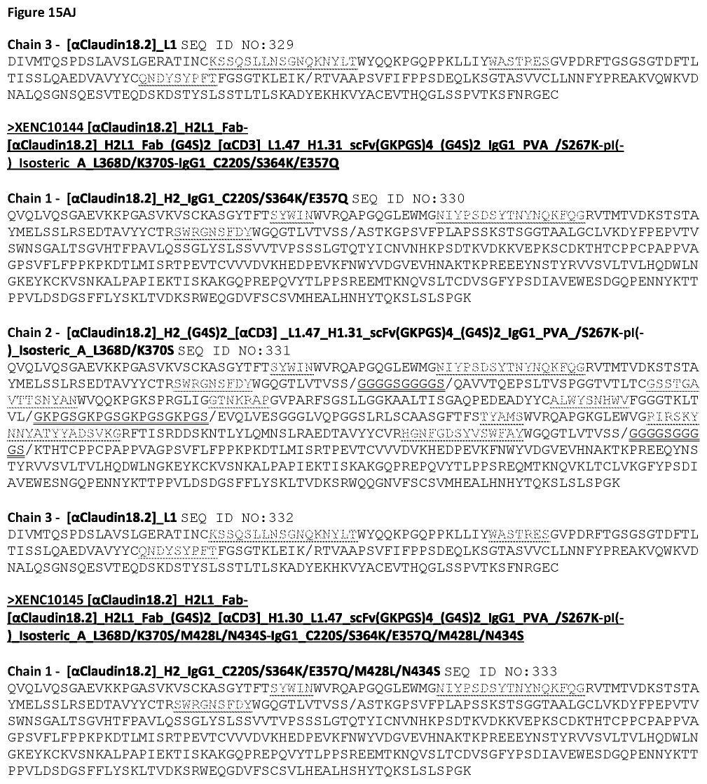

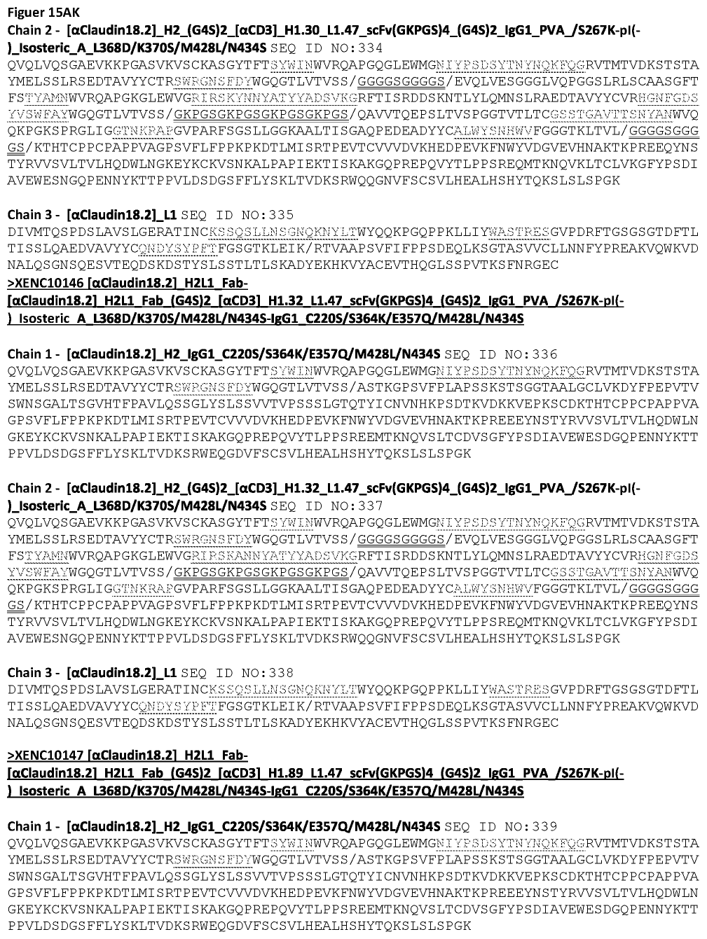

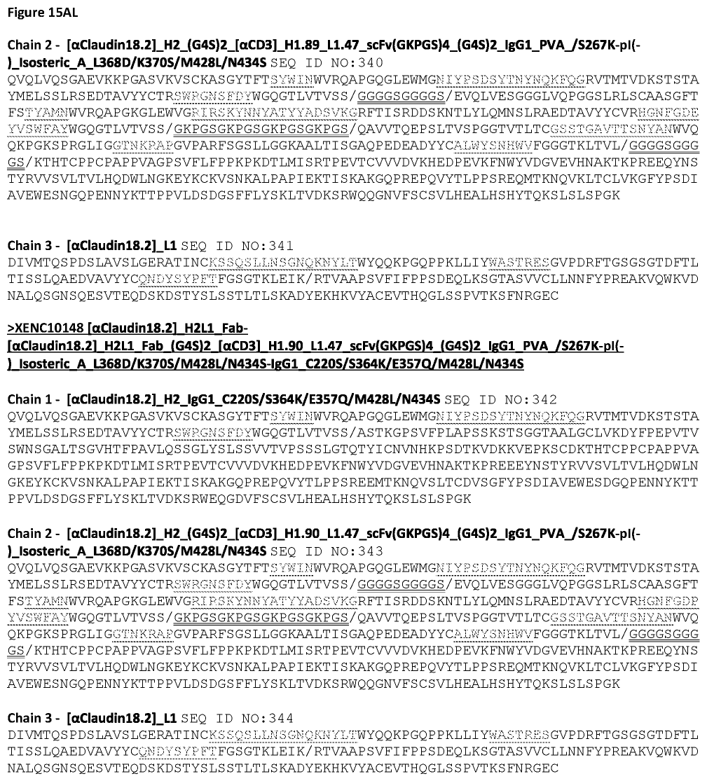

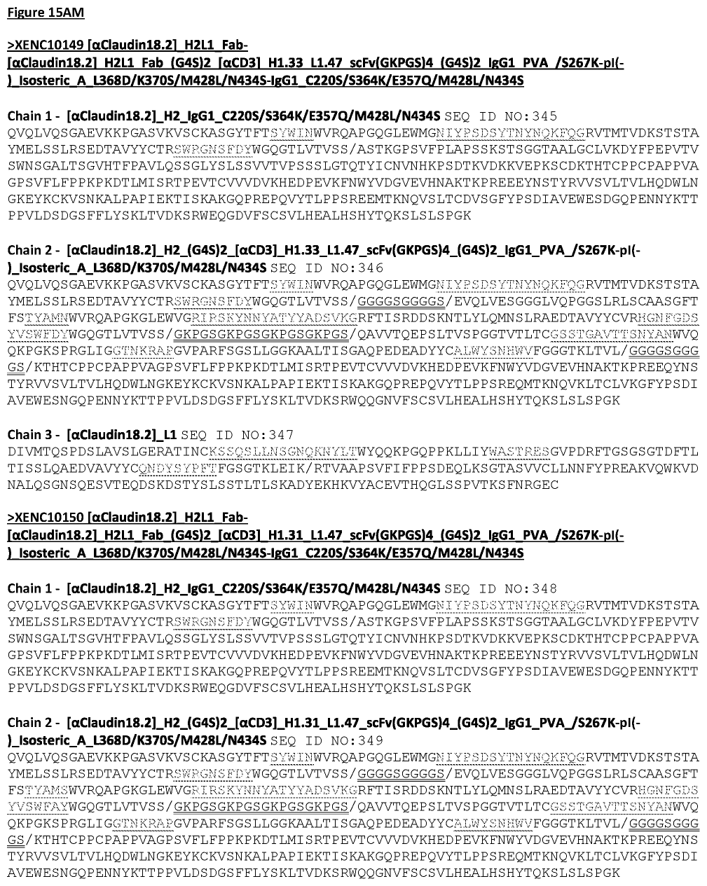

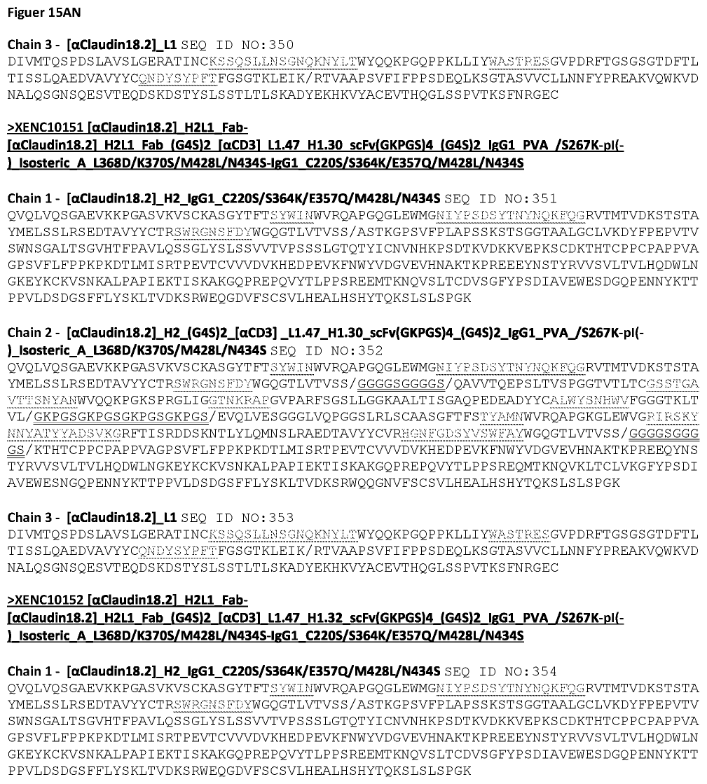

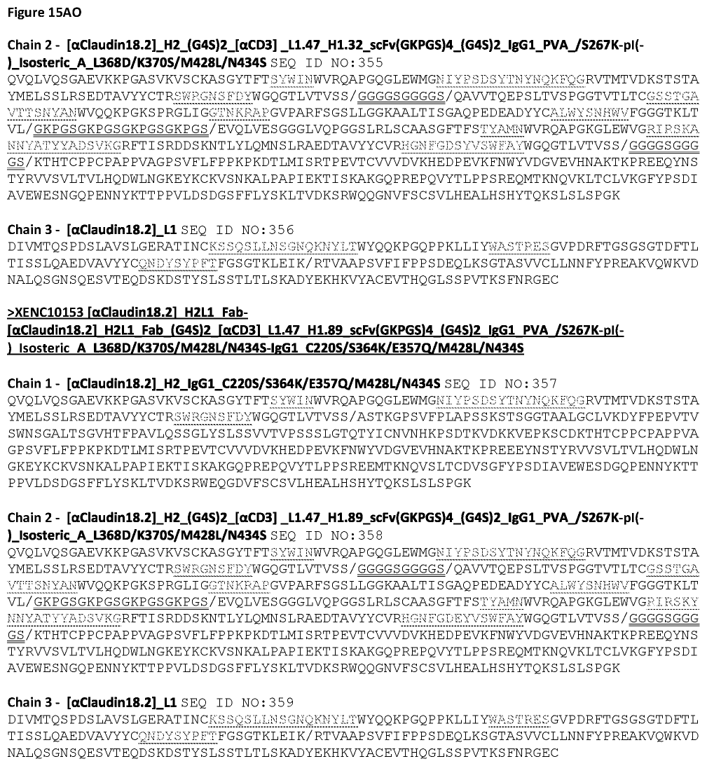

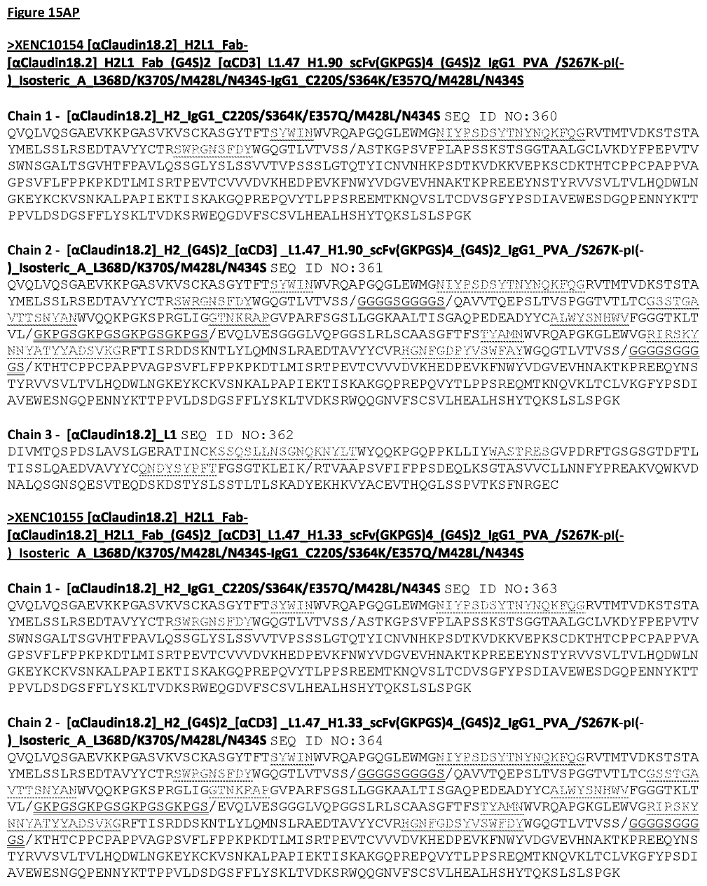

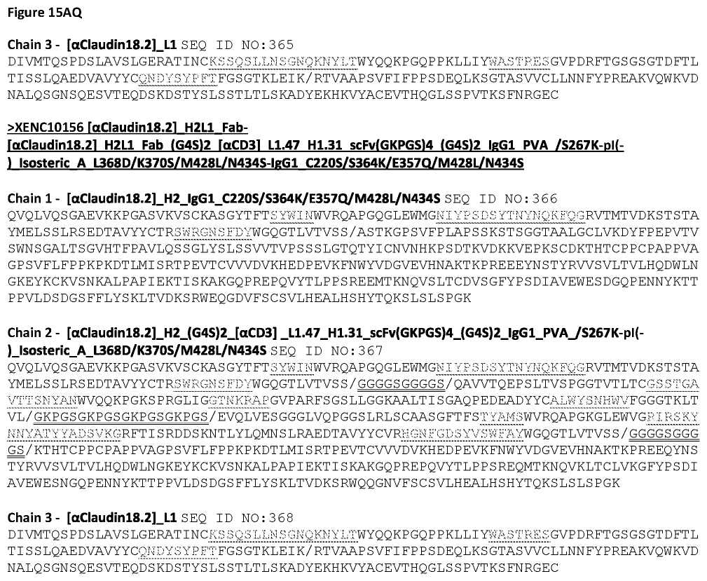

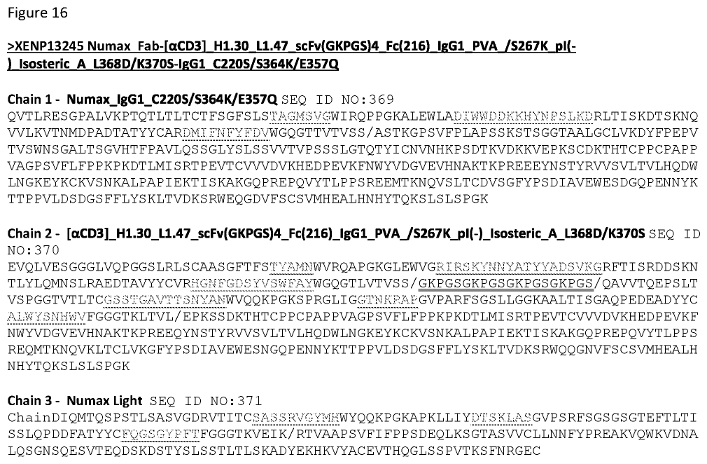

DNA encoding the anti-CLDN18.2 variable heavy chain variable and light chain variable domains was generated by gene synthesis and subcloned into a pTT5 expression vector containing appropriate fusion partners (e.g., constant regions or anti-CD3 scFv-Fc). DNA encoding anti-CD3 scFv-Fc heavy chains was generated by gene synthesis. DNA was transfected into HEK293E cells for expression. Sequences are depicted in FIGS. 14-15. An anti-RSV x anti-CD3 bispecific antibody was also generated as a control, sequences for which are depicted in FIG. 16.

(b) 1B: Binding and Redirected T Cell Cytotoxicity on NUGC-4 and KP-4 Cell Lines by Prototype Anti-CLDN18.2 x anti-CD3 bsAbs

WO 2014/075788 reported that NUGC-4 is a gastric carcinoma cell line expressing high levels of CLDN18.2, and KP-4 is a pancreatic carcinoma is a pancreatic carcinoma cell line expressing very low levels of CLDN18.2 (comparable to a negative control cell line). Accordingly, we investigated binding and redirected T cell cytotoxicity (RTCC) on NUGC-4 and KP-4 cell lines by illustrative prototype anti-CLDN18.2 x anti-CD3 bsAbs described above as well as an anti-RSV x anti-CD3 bsAb control.

In the binding experiment, 200,000 cells (either NUGC-4 or KP-4) were incubated with indicated concentrations of anti-CLDN18.2 x anti-CD3 bsAbs for an hour on ice. Samples were then washed and stained with Alexa Fluor® 647 AffiniPure F(ab′)₂ Fragment Goat Anti-Human IgG, Fcγ Fragment Specific secondary antibody (Jackson ImmunoResearch, West Grove, Penn.) for an hour on ice. Samples were washed again and analyzed by flow cytometry. Data depicting binding to KP-4 and NUGC-4 cells are depicted in FIG. 17.

In the experiment investigating RTCC, NUGC-4 or KP-4 were incubated with human PBMCs (10:1 effector to target cell ratio) and indicated concentrations of anti-CLDN18.2 x anti-CD3 bsAbs for 24 hours at 37° C. RTCC was determined using CytoTox-ONE™ Homogeneous Membrane Integrity Assay (Promega, Madison, Wis.) to measure lactate dehydrogenase levels according to manufacturer's instructions and data was acquired on a Wallac Victor2 Micrplate Reader (PerkinElmer, Waltham, Mass.), data for which are depicted in FIG. 18.

Collectively, the data show that the prototype anti-CLDN18.2 x anti-CD3 bsAbs dose-dependently bound to NUGC-4 cells, with close to baseline binding to KP-4 cells at all concentrations tested. Notably, bsAbs of the “2+1 Fab2-scFv-Fc” format (i.e. XENP24647, XENP24648, and XENP24949) bound much more potently to NUGC-4 cells than did bsAbs of the “1+1 Fab-scFv-Fc” format (i.e. XENP24645 and XENP24646) due to the extra avidity conveyed by the “2+1 Fab2-scFv-Fc” format. Consistent with cell-binding, the data show that the prototype anti-CLDN18.2 x anti-CD3 bsAbs dose-dependently induced RTCC on NUGC-4, and no RTCC on KP-4 cells.

(c) 1C: Further Characterization of Prototype Anti-CLDN18.2 x Anti-CD3 bsAbs Using NUGC-4 and SNU-601 Cells

### (i) 1C(a): Identification of SNU-601 as CLDN18.2-Expressing Cell Line

In order to better characterize the anti-CLDN18.2 x anti-CD3 bsAbs of the invention, we sought to identify cell lines with higher CLDN18.2 expression levels than NUGC-4. We investigated the binding of XENP24647 to various cell lines using flow cytometry as follows. 200,000 cells from various cell lines (and Ramos as negative control) were incubated with XENP24647 for an hour on ice, followed by staining with PE AffiniPure F(ab′)₂ Fragment Goat Anti-Human IgG, Fcγ Fragment Specific secondary antibody (Jackson ImmunoResearch, West Grove, Penn.) for an hour on ice, and analyzed by flow cytometry. FIG. 19 depicts the binding of XENP24647 to NUGC-4 and SNU-601. The data indicate that SNU-601 expresses more CLDN18.2 than does NUGC-4. Accordingly, we further characterized prototype anti-CLDN18.2 x anti-CD3 bsAbs using NUGC-4 and SNU-601 cells.

(ii) 1C(b): Binding of Prototype Anti-CLDN18.2 x Anti-CD3 bsAbs on NUGC-4 and SNU-601 Cells

We investigated the binding of prototype anti-CLDN18.2 x anti-CD3 bsAbs on NUGC-4 and SNU-601 cells as generally described above. In particular, 200,000 cells from various cell lines (and Ramos as negative control) were incubated with indicated concentrations of indicated test articles for an hour on ice, followed by staining with PE AffiniPure F(ab′)₂ Fragment Goat Anti-Human IgG, Fcγ Fragment Specific secondary antibody (Jackson ImmunoResearch, West Grove, Penn.) for an hour on ice, and analyzed by flow cytometry. PE MFI indicating binding by the bsAbs to NUGC-4 and SNU-601 are depicted in FIG. 20. Each of the bsAbs dose-dependently bound to both NUGC-4 and SNU-601, with higher maximal binding to SNU-601 cells than to NUGC-4, which is consistent with the respective CLDN18.2 expression levels on each cell line. Consistent with Example 1B, the data show that the bsAbs in the “2+1 Fab2-scFv-Fc” format bound cells more potently than bsAbs in the “1+1 Fab-scFv-Fc” format.

(iii) 1C(c): Induction of Redirected T Cell Cytotoxicity on NUGC-4 and SNU-601 Cells by Prototype anti-CLDN18.2 x Anti-CD3 bsAbs

Next, we investigated RTCC by the prototype anti-CLDN18.2 x anti-CD3 bsAbs on NUGC-4 and SNU-601 as generally described above. In particular, NUGC-4 or SNU-601 cells were incubated with human PBMCs (20:1 effector to target cell ratio) and indicated concentrations of the indicated test articles and anti-CD107a-BV421 for 24 hours or 48 hours at 37° C. Control conditions included target cells only or target cells and effector cells without test articles. After incubation, samples were stained with the following antibodies for 1 hour on ice: anti-CD69-PE, anti-CD25-APC, anti-CD4-APC-Fire750, and anti-CD8-BV605. Cells were then stained with BUV395 Annexin-V (BD Bioscience, San Jose, Calif.) and 7-AAD Viability Staining Solution (BioLegend, San Diego, Calif.). Finally, cells were analyzed by flow cytometry.

AnnexinV and 7AAD binding are indicators of the induction of apoptosis. Accordingly, live cells were AnnexinV-7AAD-, while dead cells were a sum of AnnexinV+, 7AAD+, and AnnexinV+7AAD+ cells. Data depicting number of dead/dying target cells and live target cells following incubation with effector cells and indicated test articles are depicted in FIGS. 21-24. The data show that the bsAbs enhance killing of target cells (i.e. NUGC-4 and SNU-601 cells) as indicated by increase in dead/dying cells and well as decrease in live cells. Notably, the data show that higher affinity anti-CD3 arms enhance RTCC (for example in comparing XENP24645 and XENP24646; and in comparing XENP24647 and XENP24649). Further, in the case of bsAbs having the same anti-CD3 arm, bsAbs in the “2+1 Fab2-scFv-Fc” format enhance RTCC more potently than bsAbs in the “1 +1 Fab-scFv-Fc” format (for example in comparing XENP24647 and XENP24645). Additionally, the enhanced RTCC by “2+1 Fab2-scFv-Fc” bsAbs over “1+1 Fab-scFv-Fc” bsAbs (e.g. XENP24645 and XENP24647 which are respectively anti-CLDN18.2 x anti-CD3 bsAbs in the “1+1 Fab-scFv-Fc” and “2+1 Fab2-scFv-Fc” format, and having the same affinity for CD3) is more pronounced in SNU-601 which expresses higher levels of CLDN18.2, suggesting that the “2+1 Fab2-scFv-Fc” format provides selectivity for cell lines expressing higher levels of CLDN18.2.

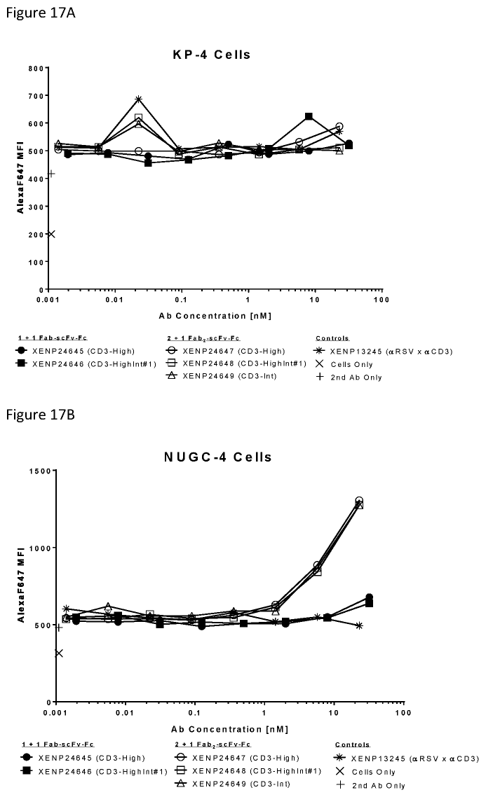

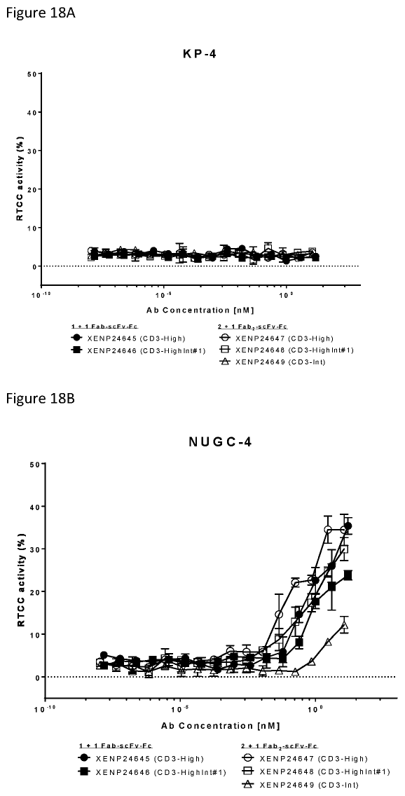

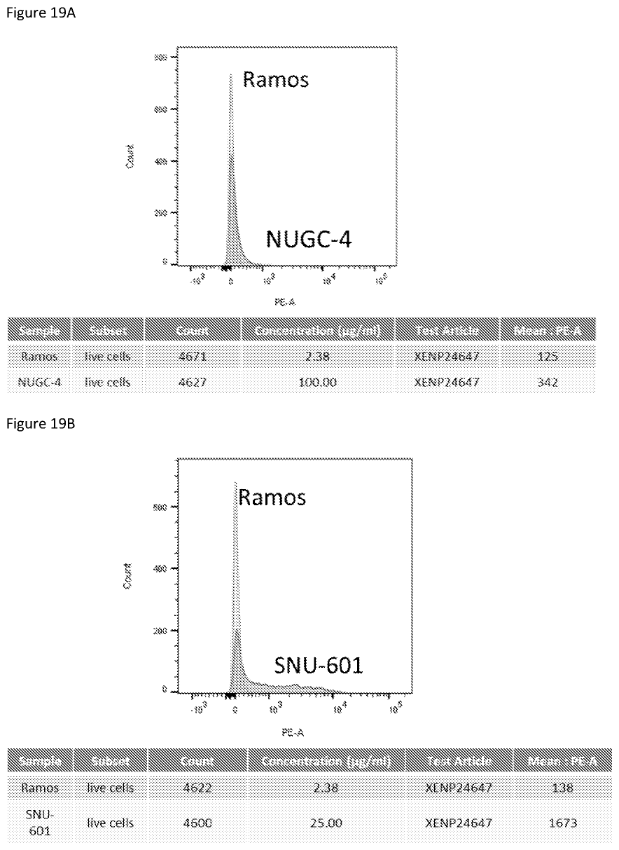

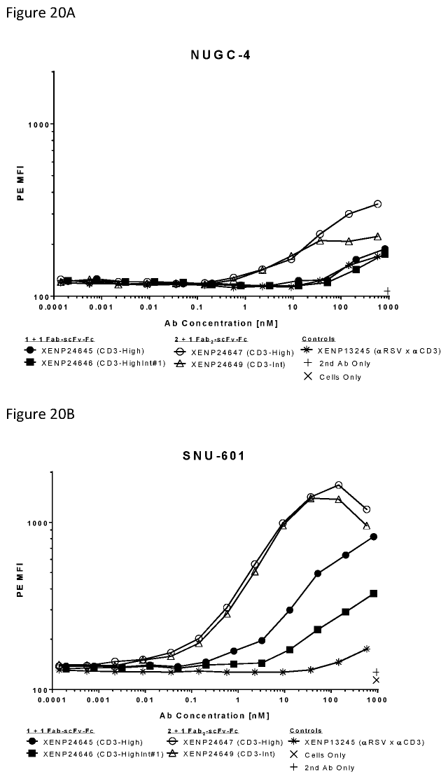

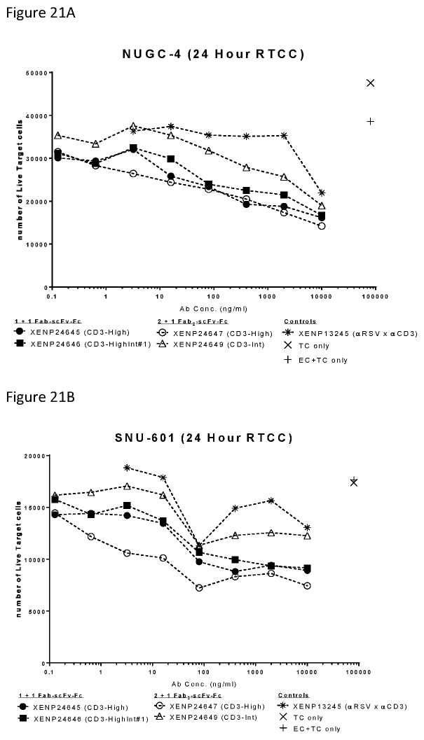

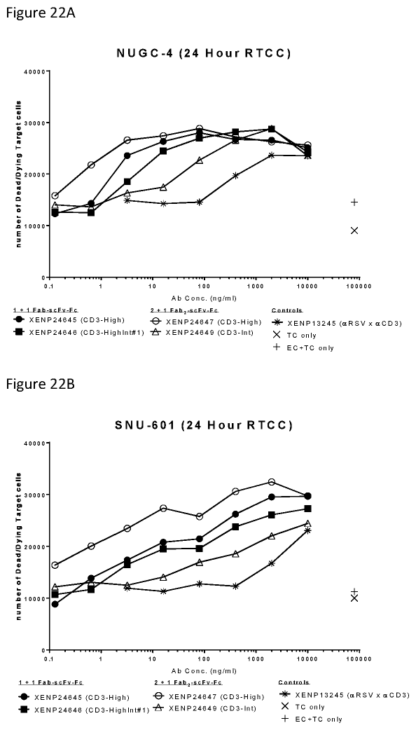

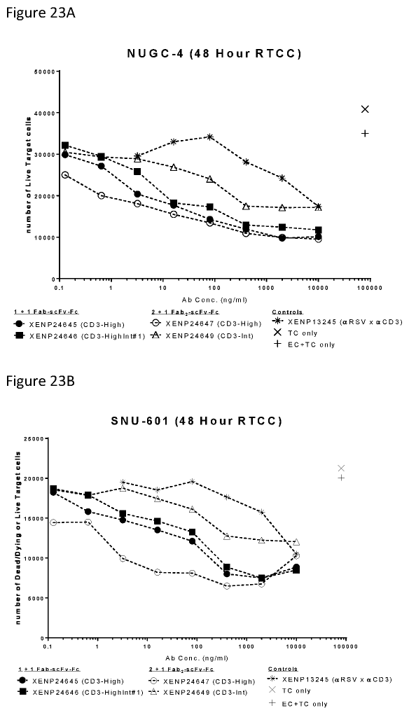

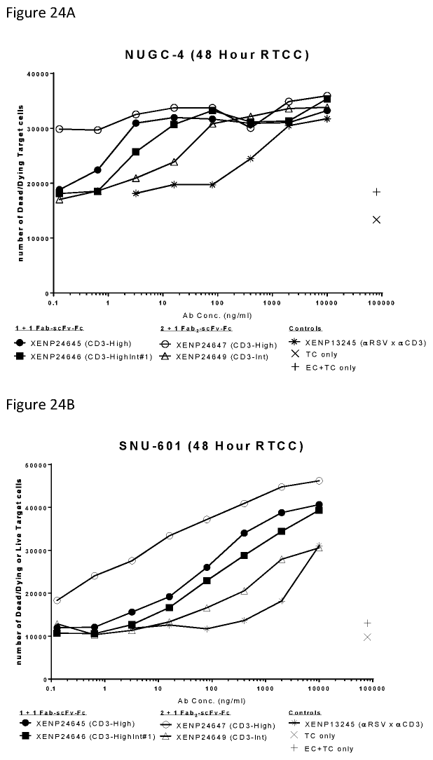

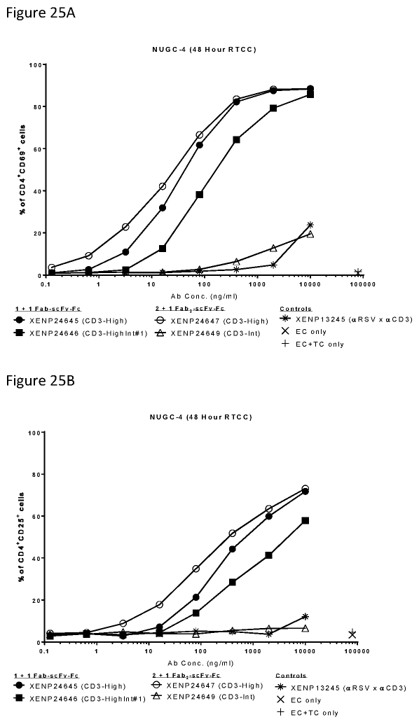

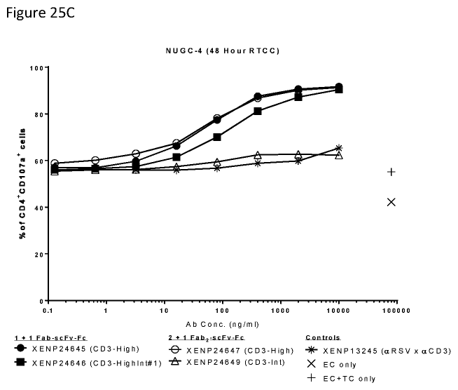

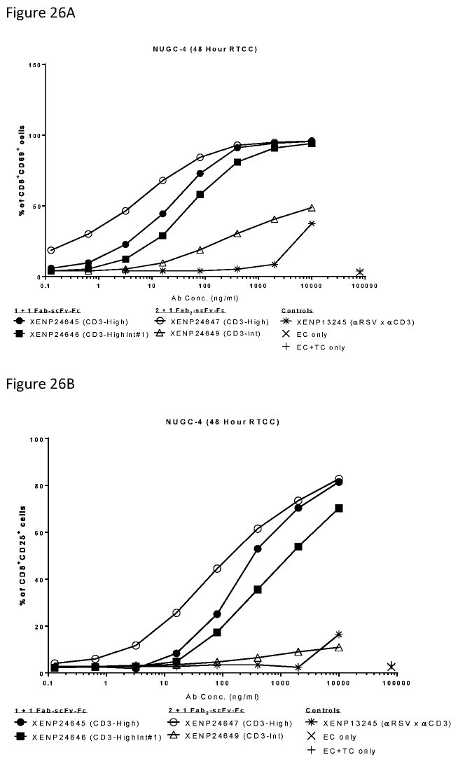

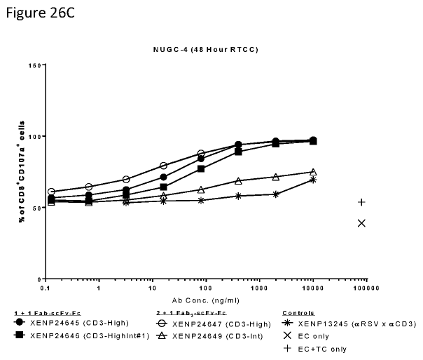

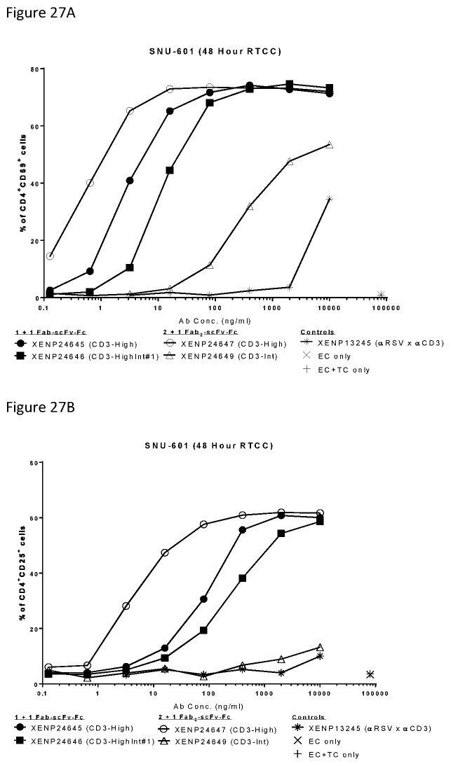

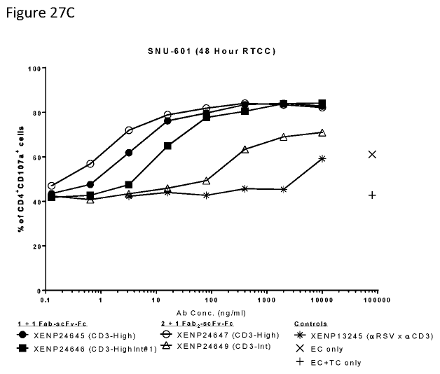

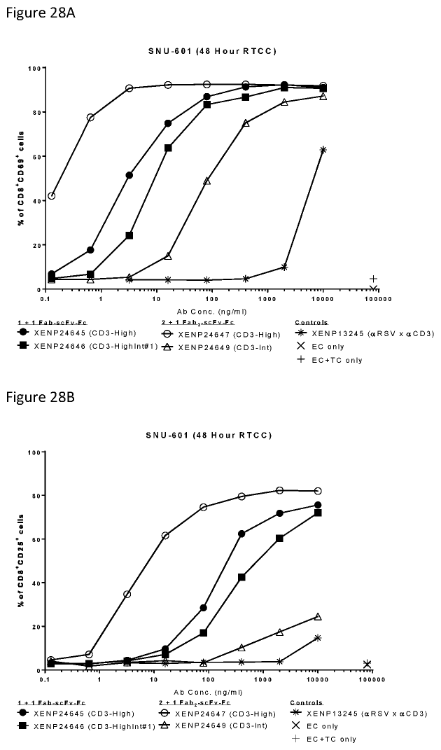

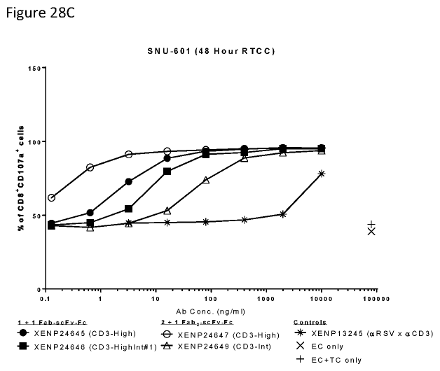

We also investigated the activation of T cells by the bsAbs of the invention. CD69 is an early activation marker, and CD25 is a late activation marker, both of which are upregulated following T cell activation via TCR signaling. CD107a is a functional marker for T cell degranulation and cytotoxic activity. Effector cells were gated to identify CD4+ and CD8+ T cells. T cells were then gated for CD69, CD25, and CD107a expression. Data depicting upregulation of the markers on CD4+ and CD8+ T cells are depicted in FIGS. 25-28. The data shows a trend consistent with RTCC, that is for example, higher affinity CD3 binding and/or bivalent CLDN18.2 binding enhance T cell activation and degranulation.

### 2.2 Example 2: Generation of Anti-CLDN18.2 x Anti-CD3 bsAbs Having Humanized CLDN18.2 Antigen Binding Domains

### (a) 2A: Humanization of CLDN18.2 Antigen Binding Domains

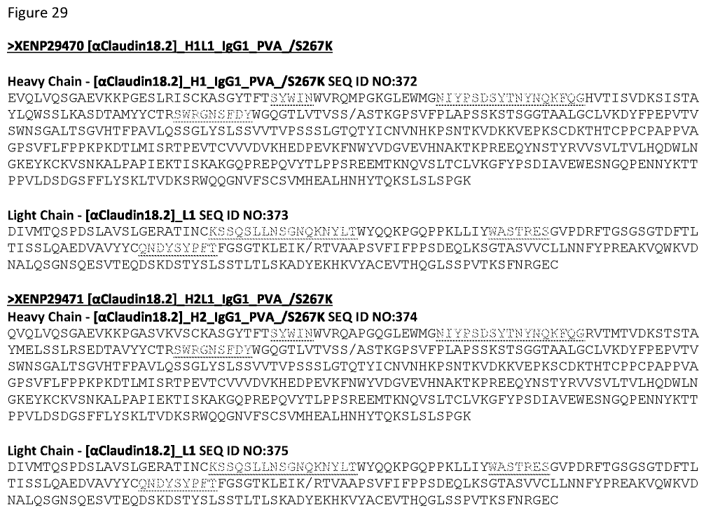

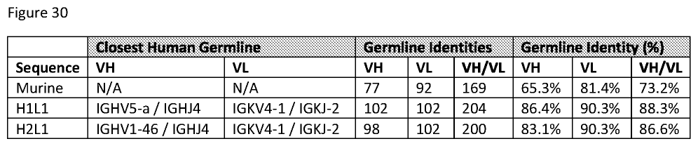

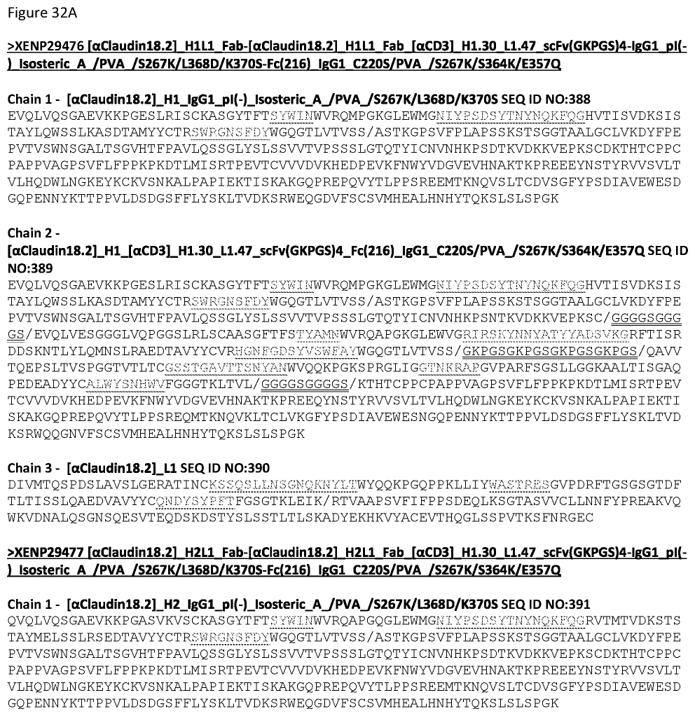

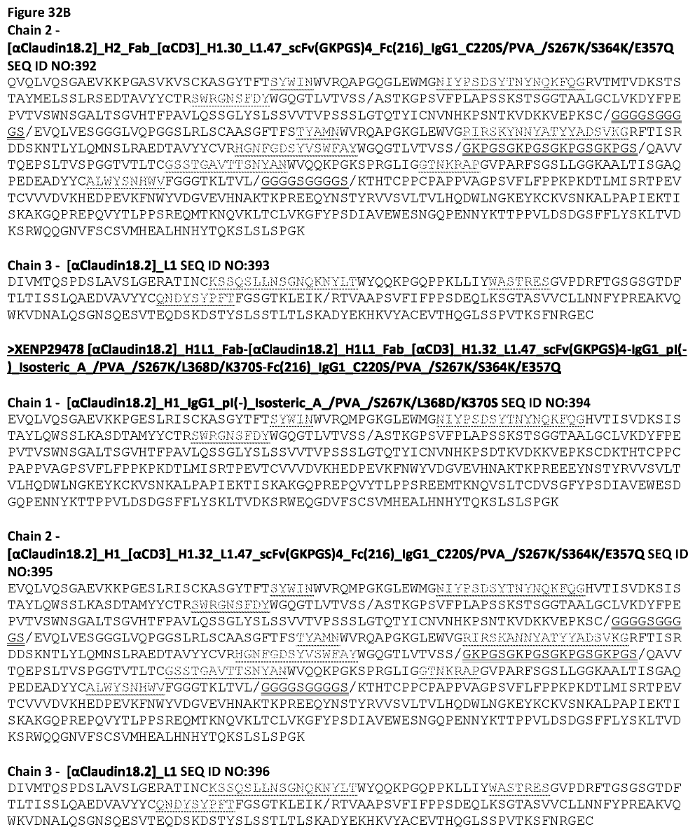

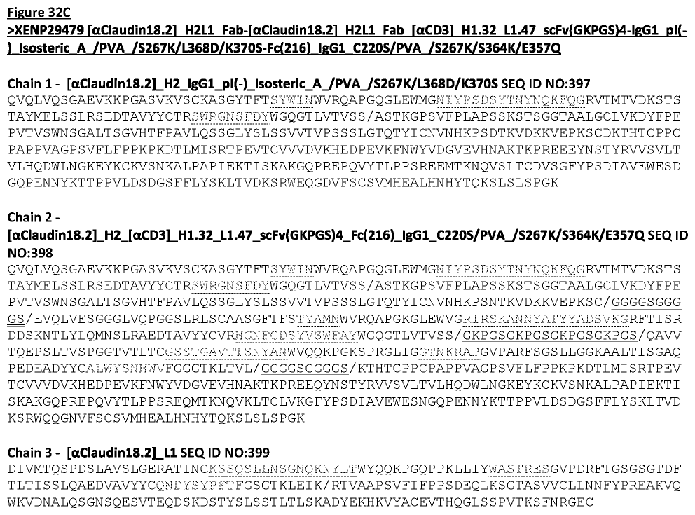

We engineered two anti-CLDN18.2 mAbs having humanized variable regions using string content optimization (see, e.g., U.S. Pat. No. 7,657,380, issued Feb. 2, 2010), sequences for which are depicted in FIG. 29. Human germline identity was increased from 73.2% for the murine ABD to 86.6% and 88.3% respectively for the two variants (see FIG. 30). The variable regions from the humanized mAbs were used to generate additional anti-CLDN18.2 x anti-CD3 bsAbs in both the “1+1 Fab-scFv-Fc” and “2+1 Fab2-scFv-Fc” format as generally described in Example 1A, sequences for which are depicted in FIGS. 31-32.

### (b) 2B: Humanization of CLDN18.2 ABD Preserved Binding, RTCC, and T Cell Activation

Next, we compared cell binding, induction of RTCC, and induction of T cell activation by anti-CLDN18.2 x anti-CD3 bsAbs having murine CLDN18.2 ABDs and having humanized CLDN18.2 ABDs.

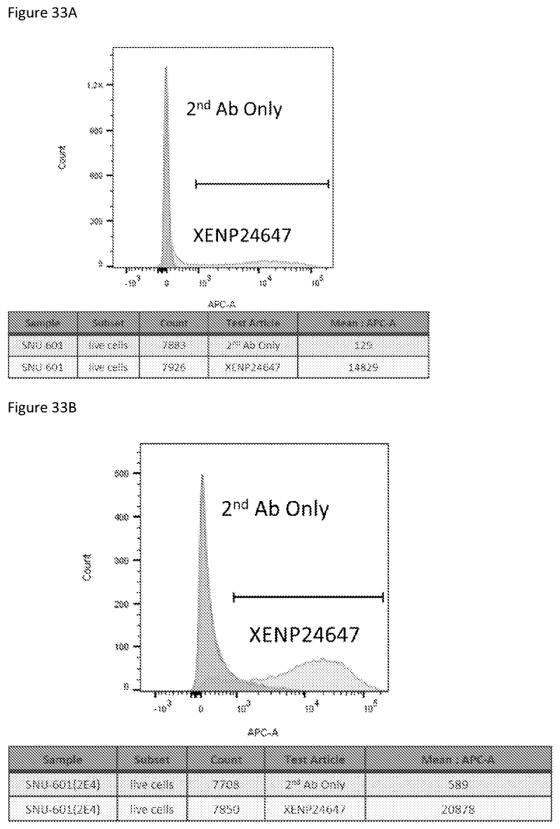

As observed in FIG. 19, although the SNU-601 cell line was characterized by higher levels of CLDN18.2 expression than the NUGC-4 cell line, the SNU-601 cell population was very heterogeneous for CLDN18.2 expression. Accordingly, using flow cytometry, we sorted SNU-601 cells based on CLDN18.2 expression and expanded CLDN18.2+ population for 4 days. The expanded CLDN18.2+ population (hereon referred to as enriched SNU-601 or SNU-601(2E4)) was re-assessed for CLDN18.2 expression as described in Example 1C(a) and compared to non-enriched SNU-601 cells, data for which are depicted in FIG. 33. The data show that the SNU-601(2E4) contains substantially higher population of CLDN18.2+ cells. Experiments in this section are performed using SNU-601(2E4) cells.

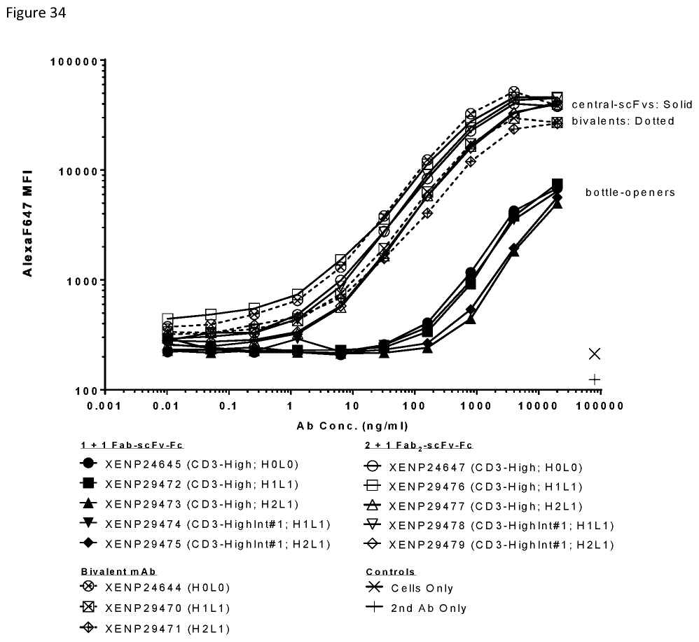

Binding by the bsAbs having humanized CLDN18.2 ABDs to SNU-601(2E4) cells were assessed as generally described in Example 1B. In particular, 200,000 SNU-601(2E4) cells were incubated with indicated concentrations of indicated test articles for an hour on ice. Samples were then stained with Alexa Fluor® 647 AffiniPure F(ab′)₂ Fragment Goat Anti-Human IgG, Fcγ Fragment Specific secondary antibody (Jackson ImmunoResearch, West Grove, Penn.), and analyzed by flow cytometry. Control samples included bsAbs having murine CLDN18.2 ABDs, bivalent anti-CLDN18.2 mAbs, cells only, and secondary antibody only. AlexaF657 MFI indicating binding by the test articles are depicted in FIG. 34. The data show that the bsAbs having humanized CLDN18.2 ABDs had similar binding to SNU-601(2E4) cells as bsAbs having murine CLDN18.2 ABDs indicating that humanization preserved the binding efficacy of the bsAbs. Notably, bsAbs in the “2+1 Fab2-scFv-Fc” format showed similar binding to bivalent anti-CLDN18.2 mAbs. Additionally, the data show that bsAbs based on the H1L1 humanized variant (e.g. XENP29472, XENP29474, XENP28476, and XENP29478) better preserved binding than did bsAbs based on the H2L1 humanized variant (e.g. XENP29473, XENP29475, XENP29477, and XENP29479).

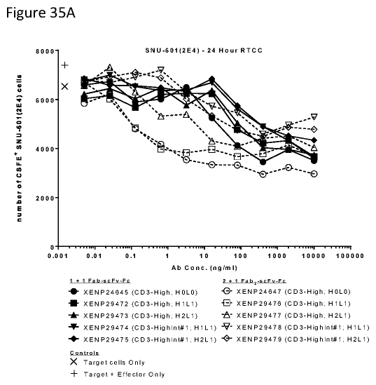

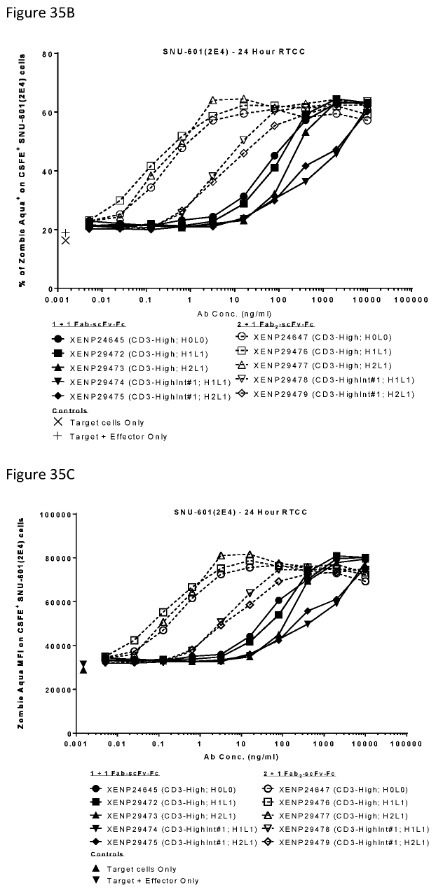

Next, we investigated induction of RTCC on SNU-601(2E4) cells and T cell activation by the bsAbs having humanized CLDN18.2 ABDs. SNU-601(2E4) cells were labeled with CellTrace™ CFSE Cell Proliferation Kit (ThermoFisher Scientific, Waltham, Mass.). CFSE-labeled SNU-601(2E4) cells were incubated with human PBMCs (10:1 effector to target ratio) and anti-CD107a-BV421 for 24 hours at 37° C. After incubation, supernatant was collected for cytokine analysis by V-PLEX Proinflammatory Panel 1 Human Kit (according to manufacturer protocol; Meso Scale Discovery, Rockville, Md.) and cells were stained with Aqua Zombie stain for 15 minutes at room temperature. Cells were then washed and stained with anti-CD4-APC-Cy7, anti-CD8-perCP-Cy5.5, and anti-CD69-APC staining antibodies, and analyzed by flow cytometry. We used two different approaches for investigating induction of RTCC: a) decrease in the number of CSFE+ target cells (data for which are depicted in FIG. 35A), and b) Zombie Aqua staining on CSFE+ target cells (data for which are depicted in FIGS. 35B-C). We also investigated activation and degranulation of CD4+ and CD8+ T cells based on CD69 and CD107a expression (data for which are depicted in FIGS. 36-39). Finally, we investigated the secretion of IFNγ and TNFα by T cells, data for which are depicted in FIG. 40. Consistent with the binding data, humanization preserved the induction of RTCC and T cell activation and cytokine secretion by the bsAbs having humanized CLDN18.2 ABDs. Notably consistent with Example 1C(c), higher affinity CD3 binding and/or bivalent CLDN18.2 binding enhance potency of RTCC as well as T cell activation, degranulation, and cytokine secretion.

What is claimed is:

1. A heterodimeric antibody comprising:
a) a first monomer comprising, from N- to C-terminal, VH-CH1-domain linker-scFv-domain linker-CH2-CH3, wherein said CH2-CH3 is a first variant Fc domain;
b) a second monomer comprising, from N- to C-terminal, VH-CH1-hinge-CH2-CH3, wherein said CH2-CH3 is a second variant Fc domain; and
c) a common light chain comprising VL-CL;
wherein one of said variant Fc domains comprises amino acid substitutions N208D/Q295E/N384D/Q418E/N421D,
wherein said first and second variant Fc domains each comprise amino acid substitutions E233P/L234V/L235A/G236del/S267K,
wherein one of said variant Fc domains comprises amino acid substitutions S364K/E357Q and the other variant Fc domain comprises amino acid substitutions L368D/K370S,
wherein said scFv has a sequence selected from the group consisting of SEQ ID NO:87, SEQ ID NO:88, SEQ ID NO:98, SEQ ID NO:99, SEQ ID NO:109, SEQ ID NO:110, SEQ ID NO:120, SEQ ID NO:121, SEQ ID NO:131, SEQ ID NO:132, SEQ ID NO:142, SEQ ID NO:143;
wherein said VH domain comprises SEQ ID NO:81 or SEQ ID NO:82 and said VL domain comprises SEQ ID NO:84, wherein numbering is according to the EU index as in Kabat.

2. A heterodimeric antibody according to claim 1 selected from the group consisting of XENP24647, XENP31729, XENP24649, XENP31723, XENP31725, XENP31727, XENP29476, XENP29478, XENP31724, XENP31726, XENP31728, XENP29477, XENP29479, XENC10101, XENC10102, XENC10103, XENC10104, XENC10105, XENC10106, XENC10107, XENC10108, XENC10109, XENC10110, XENC10111, XENC10112, XENC10113, XENC10114, XENC10115, XENC10116, XENC10117, XENC10118, XENC10119, XENC10120, XENC10121, XENC10122, XENC10123, XENC10124, XENC10125, XENC10126, XENC10127, XENC10128, XENC10129, XENC10130, XENC10131, XENC10132, XENC10133, XENC10134, XENC10135, XENC10136, XENC10137, XENC10138, XENC10139, XENC10140, XENC10141, XENC10142, XENC10143, XENC10144, XENC10145, XENC10146, XENC10147, XENC10148, XENC10149, XENC10150, XENC10151, XENC10152, XENC10153, XENC10154, XENC10155 and XENC10156.

3. A composition comprising an anti-CLDN18.2 antigen binding domain (ABD), wherein said ABD comprises:
a) a variable heavy domain comprising SEQ ID NO:81; and
b) a variable light domain comprising SEQ ID NO:84.

4. A composition comprising an anti-CLDN18.2 antigen binding domain (ABD), wherein said ABD comprises:
a) a variable heavy domain comprising SEQ ID NO:82; and
b) a variable light domain comprising SEQ ID NO:84.

5. A nucleic acid composition comprising:
a) a first nucleic acid encoding a variable heavy domain selected from the group consisting of SEQ ID NO:81 and SEQ ID NO:82; and
b) a second nucleic acid encoding SEQ ID NO:84.

6. An expression vector comprising said first and second nucleic acids according to claim 5.

7. An expression vector composition comprising:
a) a first expression vector comprising said first nucleic acid of claim 5; and
b) a second expression vector comprising said second nucleic acid of claim 5.

8. A host cell comprising an expression vector according to claim 6 or an expression vector composition according to claim 7.

9. A method of producing an anti-CLDN18.2 ABD comprising culturing the host cell of claim 8 under conditions where said ABD is produced and recovering said ABD.

10. A heterodimeric antibody comprising:
a) a first monomer comprising, from N- to C-terminal, scFv-domain linker-CH2-CH3, wherein said CH2-CH3 is a first variant Fc domain;
b) a second monomer comprising, from N- to C-terminal, VH-CH1-hinge-CH2-CH3, wherein said CH2-CH3 is a second variant Fc domain; and
c) a third monomer comprising VL-CL;
wherein one of said variant Fc domains comprises amino acid substitutions N208D/Q295E/N384D/Q418E/N421D,
wherein said first and second variant Fc domains each comprise amino acid substitutions E233P/L234V/L235A/G236del/S267K,
wherein one of said variant Fc domains comprises amino acid substitutions S364K/E357Q and the other variant Fc domain comprises amino acid substitutions L368D/K370S,
wherein said scFv has a sequence selected from the group consisting of SEQ ID NO:87, SEQ ID NO:88, SEQ ID NO:98, SEQ ID NO:99, SEQ ID NO:109, SEQ ID NO:110, SEQ ID NO:120, SEQ ID NO:121, SEQ ID NO:131, SEQ ID NO:132, SEQ ID NO:142, SEQ ID NO:143;
wherein said variable heavy (VH) domain comprises SEQ ID NO:81 or SEQ ID NO:82 and said variable light (VL) domain comprises SEQ ID NO:84, wherein numbering is according to the EU index as in Kabat.

11. A heterodimeric antibody according to claim 10, wherein the CH1-hinge-CH2-CH3 component of the second heavy chain comprises SEQ ID NO:32, said first variant Fc domain comprises SEQ ID NO:33 and said constant light domain comprises SEQ ID NO:74.

12. A heterodimeric antibody according to claim 10 selected from the group consisting of XENP29472, XENP29473, XENP29474 and XENP29475.

13. A nucleic acid composition comprising:
a) a first nucleic acid encoding said first heavy chain of claim 10;
b) a second nucleic acid encoding said second heavy chain of claim 10; and
c) a third nucleic acid encoding said light chain of claim 10.

14. An expression vector composition comprising:
a) a first expression vector comprising said first nucleic acid of claim 13;
b) a second expression vector comprising said second nucleic acid of claim 13; and
c) a third expression vector comprising said third nucleic acid of claim 13.

15. A host cell comprising said expression vector composition of claim 14.

16. A method of making a heterodimeric antibody according to claim 10 comprising culturing said host cell of claim 15 under conditions wherein said antibody is expressed, and recovering said antibody.

17. A method of treating a gastric cancer in a subject in need thereof, comprising administering to said subject a heterodimeric antibody according to claim 10.

18. A heterodimeric antibody comprising:
a) a first monomer comprising, from N- to C-terminal, VH1-CH1-hinge-CH2-CH3-domain linker-VH2, wherein said CH2-CH3 is a first variant Fc domain;
b) a second monomer comprising, from N- to C-terminal, VH1-CH1-hinge-CH2-CH3-domain linker-VL2, wherein said CH2-CH3 is a second variant Fc domain; and
c) a common light chain comprising a VL1-CL;
wherein one of said variant Fc domains comprises amino acid substitutions N208D/Q295E/N384D/Q418E/N421D,
wherein said first and second variant Fc domains each comprise amino acid substitutions E233P/L234V/L235A/G236del/S267K;
wherein one of said variant Fc domains comprises amino acid substitutions S364K/E357Q and the other Fc domain comprises amino acid substitutions L368D/K370S;
wherein said VH2 and VL2 are selected from the pairs consisting of SEQ ID NO:89 and SEQ ID NO:93, SEQ ID NO:100 and SEQ ID NO:104, SEQ ID NO:111 and SEQ ID NO:115, SEQ ID NO:122 and SEQ ID NO:126, SEQ ID NO: 133 and SEQ ID NO:137, SEQ ID NO:144 and SEQ ID NO:148;
wherein said VH1 domain comprises SEQ ID NO:81 or SEQ ID NO:82 and said VL1 domain comprises SEQ ID NO:84, wherein numbering is according to the EU index as in Kabat.

19. A heterodimeric antibody comprising:
a) a first monomer comprising, from N- to C-terminal, VH1-CH1-hinge-CH2-CH3-domain linker-scFv, wherein said CH2-CH3 is a first variant Fc domain;
b) a second monomer comprising, from N- to C-terminal, VH1-CH1-hinge-CH2-CH3, wherein said CH2-CH3 is a second variant Fc domain; and
c) a common light chain comprising a VL1-CL;
wherein one of said variant Fc domains comprises amino acid substitutions N208D/Q295E/N384D/Q418E/N421D,
wherein said first and second variant Fc domains each comprise amino acid substitutions E233P/L234V/L235A/G236del/S267K;
wherein one of said variant Fc domains comprises amino acid substitutions S364K/E357Q and the other Fc domain comprises amino acid substitutions L368D/K370S;
wherein said scFv has a sequence selected from the group consisting of SEQ ID NO:87, SEQ ID NO:88, SEQ ID NO:98, SEQ ID NO:99, SEQ ID NO:109, SEQ ID NO:110, SEQ ID NO:120, SEQ ID NO:121, SEQ ID NO:131, SEQ ID NO:132, SEQ ID NO:142, SEQ ID NO:143; and wherein said VH1 domain comprises SEQ ID NO:81 or SEQ ID NO:82 and said VL1 domain comprises SEQ ID NO:84, wherein numbering is according to the EU index as in Kabat.

20. A heterodimeric antibody comprising:
a) a first monomer comprising, from N- to C-terminal, VH1-CH1-domain linker-VH2-domain linker-CH2-CH3, wherein said CH2-CH3 is a first variant Fc domain;
b) a second monomer comprising, from N- to C-terminal, VH1-CH1-domain linker-VL2-domain linker-CH2-CH3, wherein said CH2-CH3 is a second variant Fc domain; and
c) a common light chain comprising a VL1-CL;
wherein one of said variant Fc domains comprises amino acid substitutions N208D/Q295E/N384D/Q418E/N421D,
wherein said first and second variant Fc domains each comprise amino acid substitutions E233P/L234V/L235A/G236del/S267K;
wherein one of said variant Fc domains comprises amino acid substitutions S364K/E357Q and the other Fc domain comprises amino acid substitutions L368D/K370S;
wherein said VH2 and VL2 are selected from the pairs consisting of SEQ ID NO:89 and SEQ ID NO:93, SEQ ID NO:100 and SEQ ID NO:104, SEQ ID NO:111 and SEQ ID NO:115, SEQ ID NO:122 and SEQ ID NO:126, SEQ ID NO: 133 and SEQ ID NO:137, SEQ ID NO:144 and SEQ ID NO:148;
wherein said VH1 domain comprises SEQ ID NO:81 or SEQ ID NO:82 and said VL1 domain comprises SEQ ID NO:84, wherein numbering is according to the EU index as in Kabat.

21. A heterodimeric antibody comprising:
a) a first monomer comprising, from N- to C-terminal, VH-CH1-domain linker-scFv-domain linker-CH2-CH3, wherein said CH2-CH3 is a first variant Fc domain;
b) a second monomer comprising a second variant Fc domain comprising CH2-CH3; and
c) a light chain comprising VL-CL;
wherein one of said variant Fc domains comprises amino acid substitutions N208D/Q295E/N384D/Q418E/N421D,
wherein said first and second variant Fc domains each comprise amino acid substitutions E233P/L234V/L235A/G236del/S267K,
wherein one of said variant Fc domains comprises amino acid substitutions S364K/E357Q and the other variant Fc domain comprises amino acid substitutions L368D/K370S,
wherein said scFv has a sequence selected from the group consisting of SEQ ID NO:87, SEQ ID NO:88, SEQ ID NO:98, SEQ ID NO:99, SEQ ID NO:109, SEQ ID NO:110, SEQ ID NO:120, SEQ ID NO:121, SEQ ID NO:131, SEQ ID NO:132, SEQ ID NO:142, SEQ ID NO:143;
wherein said VH domain comprises SEQ ID NO:81 or SEQ ID NO:82 and said VL domain comprises SEQ ID NO:84, wherein numbering is according to the EU index as in Kabat.

22. A heterodimeric antibody comprising:
a) a first monomer comprising, from N- to C-terminal, scFv-domain linker-VH-CH1-hinge-CH2-CH3, wherein said CH2-CH3 is a first variant Fc domain;
b) a second monomer comprising a second variant Fc domain comprising CH2-CH3; and
c) a light chain comprising VL-CL;
wherein one of said variant Fc domains comprises amino acid substitutions N208D/Q295E/N384D/Q418E/N421D,
wherein said first and second variant Fc domains each comprise amino acid substitutions E233P/L234V/L235A/G236del/S267K,
wherein one of said variant Fc domains comprises amino acid substitutions S364K/E357Q and the other variant Fc domain comprises amino acid substitutions L368D/K370S,
wherein said scFv has a sequence selected from the group consisting of SEQ ID NO:87, SEQ ID NO:88, SEQ ID NO:98, SEQ ID NO:99, SEQ ID NO:109, SEQ ID NO:110, SEQ ID NO:120, SEQ ID NO:121, SEQ ID NO:131, SEQ ID NO:132, SEQ ID NO:142, SEQ ID NO:143;
wherein said VH domain comprises SEQ ID NO:81 or SEQ ID NO:82 and said VL domain comprises SEQ ID NO:84, wherein numbering is according to the EU index as in Kabat.

23. A heterodimeric antibody comprising:
a) a first monomer comprising, from N- to C-terminal, scFv-domain linker-VH-CH1-hinge-CH2-CH3, wherein said CH2-CH3 is a first variant Fc domain;
b) a second monomer comprising, from N- to C-terminal, VH-CH1-hinge-CH2-CH3, wherein said CH2-CH3 is a second variant Fc domain; and
c) a common light chain comprising VL-CL;
wherein one of said variant Fc domains comprises amino acid substitutions N208D/Q295E/N384D/Q418E/N421D,
wherein said first and second variant Fc domains each comprise amino acid substitutions E233P/L234V/L235A/G236del/S267K,
wherein one of said variant Fc domains comprises amino acid substitutions S364K/E357Q and the other variant Fc domain comprises amino acid substitutions L368D/K370S,
wherein said scFv has a sequence selected from the group consisting of SEQ ID NO:87, SEQ ID NO:88, SEQ ID NO:98, SEQ ID NO:99, SEQ ID NO:109, SEQ ID NO:110, SEQ ID NO:120, SEQ ID NO:121, SEQ ID NO:131, SEQ ID NO:132, SEQ ID NO:142, SEQ ID NO:143,
wherein said VH domain comprises SEQ ID NO:81 or SEQ ID NO:82 and said VL domain comprises SEQ ID NO:84, wherein numbering is according to the EU index as in Kabat.

24. A nucleic acid composition comprising:
a) a first nucleic acid encoding said first monomer of any of claims 1, 2, 10-12, and 18-23;
b) a second nucleic acid encoding said second monomer of any of claims 1, 2, 10-12, and 18-23, respectively; and
c) a third nucleic acid encoding said third monomer of any of claims 1, 2, 10-12, and 18-23, respectively.

25. An expression vector composition comprising:
a) a first expression vector comprising said first nucleic acid of claim 24;
b) a second expression vector comprising said second nucleic acid of claim 24; and
c) a third expression vector comprising said third nucleic acid of claim 24.

26. A host cell comprising said expression vector composition of claim 25.

27. A method of making a heterodimeric antibody according to any of claims 1, 2, 10-12, and 18-23 comprising culturing said host cell of claim 26 under conditions wherein said antibody is expressed, and recovering said antibody.

28. A method of treating a gastric cancer in a subject in need thereof, comprising administering to said subject a heterodimeric antibody according to any of claims 1, 2, 10-12, and 18-23.
